(19) United States
(12) Patent Application Publication
(10) Pub. No.: US 2004/0068767 A1
(43) Pub. Date: Apr. 8, 2004

(54) Maize cellulose synthases and uses thereof

(75) Inventors: Kanwarpal S. Dhugga, Johnston, IA (US); Haiyin Wang, Johnston, IA (US); Dwight T. Tomes, Van Meter, IA (US); Timothy G. Helentjaris, Ankeny, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc., Johnston, IA

(21) Appl. No.: 10/627,132

(22) Filed: Jul. 25, 2003

Related U.S. Application Data
(63) Continuation-in-part of application No. 10/209,059, filed on Jul. 31, 2002.
Application No. 10/209,059 is a continuation-in-part of application No. 09/550,483, filed on Apr. 14, 2000, now abandoned.
Application No. 09/550,483 is a continuation-in-part of application No. 09/371,383, filed on Aug. 6, 1999, now abandoned.
(60) Provisional application No. 60/096,822, filed on Aug. 17, 1998.

(51) Int. Cl. A01H 1/00; C12N 15/82; C12Q 1/68; C07H 21/04; C12N 9/42; C12N 5/04

(52) U.S. Cl. 800/284; 435/6; 435/69.1; 435/209; 435/419; 536/23.2

(57) ABSTRACT

The invention provides isolated cellulose synthase nucleic acids and their encoded proteins. The present invention provides methods and compositions relating to altering cellulose synthase levels in plants. The invention further provides recombinant expression cassettes, host cells, transgenic plants, and antibody compositions.

### RELATED APPLICATIONS

This application is a continuation in part of co-pending U.S. application Ser. No. 10/209,059, filed Jul. 31, 2002, which claims the benefit of U.S. application Ser. No. 09/550,483, filed Apr. 14, 2000, now abandoned, which claims benefit of U.S. application Ser. No. 09/371,383, filed Aug. 6, 1999, now abandoned, which claims benefit of U.S. Provisional Application No. 60/096,822, filed Aug. 17, 1998, now abandoned, all of which are incorporated herein by reference. This application also claims the benefit of co-pending U.S. application Ser. No. 10/267,459, filed Oct. 9, 2002, which claims the benefit of U.S. application Ser. No. 09/550,483, filed Apr. 14, 2000, now abandoned, which claims benefit of U.S. application Ser. No. 09/371,383, filed Aug. 6, 1999, now abandoned, which claims benefit of U.S. Provisional Application No. 60/096,822, filed Aug. 17, 1998, now abandoned, all of which are incorporated herein by reference. This application also claims the benefit of co-pending application Ser. No. 10/160,719, filed Jun. 3, 2002, which claims benefit of U.S. application Ser. No. 09/371,383, filed Aug. 6, 1999, now abandoned, which claims benefit of U.S. Provisional Application No. 60/096,822, filed Aug. 17, 1998, now abandoned.

### TECHNICAL FIELD

The present invention relates generally to plant molecular biology. More specifically, it relates to nucleic acids and methods for modulating their expression in plants.

### BACKGROUND OF THE INVENTION

Polysaccharides constitute the bulk of the plant cell walls and have been traditionally classified into three categories: cellulose, hemicellulose, and pectin. Fry, S. C. (1988), The growing plant cell wall: Chemical and metabolic analysis, New York: Longman Scientific & Technical. Whereas cellulose is made at the plasma membrane and directly laid down into the cell wall, hemicellulosic and pectic polymers are first made in the Golgi apparatus and then exported to the cell wall by exocytosis. Ray, P. M., et al., (1976), Ber. Deutsch. Bot. Ges. Bd. 89, 121-146. The variety of chemical linkages in the pectic and hemicellulosic polysaccharides indicates that there must be tens of polysaccharide synthases in the Golgi apparatus. Darvill et al., (1980), The primary cell walls of flowering plants. In The Plant Cell (N. E. Tolbert, ed.), Vol. 1 in Series: The biochemistry of plants: A comprehensive treatise, eds. P. K. Stumpf and E. E. Conn (New York: Academic Press), pp. 91-162.

Even though sugar and polysaccharide compositions of the plant cell walls have been well characterized, very limited progress has been made toward identification of the enzymes involved in polysaccharides formation, the reason being their labile nature and recalcitrance to solubilization by available detergents. Sporadic claims for the identification of cellulose synthase from plant sources were made over the years. Callaghan, T., and Benziman, M. (1984), Nature 311, 165-167; Okuda, et al., (1993), Plant Physiol. 101, 1131-1142. However, these claims were met with skepticism. Callaghan, T., and Benziman, M. (1985), Nature 314, 383-384; Delmer, et al., (1993), Plant Physiol. 103, 307-308. It was only relatively recently that a putative gene for plant cellulose synthase (CesA) was cloned from the developing cotton fibers based on homology to the bacterial gene. Pear, et al., Proc. Natl. Acad. Sci. (USA) 93, 12637-12642; Saxena, et al., (1990), Plant Molecular Biology 15, 673-684; see also, WO 9818949; see also Arioli, T., Peng, L., Betzner Andreas, S., Burn, J., Wittke, W., Herth, W., Camilleri, C., Hofte, H., Plazinski, J., Birch, R., Cork, A., Glover, J., Redmond, J., and Williamson Richard, E. (1998). Molecular analysis of cellulose biosynthesis in Arabidopsis. Science Washington D.C. Jan. 279, 717-720. A number of genes for cellulose synthase family were later isolated from other plant species based on sequence homology to the cotton gene (Richmond Todd, A., and Somerville Chris, R. (2000), The cellulose synthase superfamily, Plant Physiology, 2000; 124, 495-498.)

Cellulose, by virtue of its ability to form semicrystalline microfibrils, has a very high tensile strength which approaches that of some metals. Niklas, K. J. (1992), Plant Biomechanics: An engineering approach to plant form and function, The University of Chicago Press, p. 607. Bending strength of the culm of normal and brittle-culm mutants of barley has been found to be directly correlated with the concentration of cellulose in the cell wall. Kokubo, et al., (1989), Plant Physiology 91, 876-882; Kokubo, et al., (1991) Plant Physiology 97, 509-514.

Although stalk composition contributes to numerous quality factors important in maize breeding, little is known in the art about the impact of cellulose levels on such agronomically important traits as stalk lodging, silage digestibility, or downstream processing. The present invention provides these and other advantages.

### SUMMARY OF THE INVENTION

Generally, it is the object of the present invention to provide nucleic acids and proteins relating to cellulose synthases. It is an object of the present invention to provide transgenic plants comprising the nucleic acids of the present invention, and methods for modulating, in a transgenic plant, expression of the nucleic acids of the present invention.

Therefore, in one aspect the present invention relates to an isolated nucleic acid comprising a member selected from the group consisting of (a) a polynucleotide having a specified sequence identity to a polynucleotide encoding a polypeptide of the present invention; (b) a polynucleotide which is complementary to the polynucleotide of (a); and, (c) a polynucleotide comprising a specified number of contiguous nucleotides from a polynucleotide of (a) or (b). The isolated nucleic acid can be DNA.

In other aspects the present invention relates to: 1) recombinant expression cassettes, comprising a nucleic acid of the present invention operably linked to a promoter, 2) a host cell into which has been introduced the recombinant expression cassette, 3) a transgenic plant comprising the recombinant expression cassette, and 4) a transgenic plant comprising a recombinant expression cassette containing more than one nucleic acid of the present invention each operably linked to a promoter. Furthermore, the present invention also relates to combining by crossing and hybridization recombinant cassettes from different transformants. The host cell and plant are optionally from maize, wheat, rice, or soybean.

In other aspects the present invention relates to methods of altering stalk lodging and other standability traits, including, but not limited to brittle snap, and improving stalk digestibility, through the introduction of one or more of the polynucleotides that encode the polypeptides of the present invention. Additional aspects of the present invention include methods and transgenic plants useful in the end use processing of compounds such ads cellulose or use of transgenic plants as end products either directly, such as silage, or indirectly following processing, for such uses known to those of skill in the art, such as, but not limited to, ethanol. Also, one of skill in the art would recognize that the polynucleotides and encoded polypeptides of the present invention can be introduced into an host cell or transgenic plant wither singly or in multiples, sometimes referred to in the art as “stacking” of sequences or traits. It is intended that these compositions and methods be encompassed in the present invention.

### BRIEF DESCRIPTION OF THE DRAWINGS

FIG. 1: Stalk breaking strength of hybrids and its comparison with the lodging scores. The mechanical strength is very similar to the lodging scores that have been assigned based on field observations. The vertical light-colored bar in the upper right corner of the figure is the least significant difference (LSD) estimate at 5% level.

FIG. 2: Stalk strength of To transgenic plants. Plants overexpressing (“up”) ZmCesA4 and ZmCesA8 had significantly stronger stalks than the controls. Overexpression of CesA5 did not alter stalk strength whereas the overexpression of CesA1 led to weaker and stunted stalks.

FIG. 3: Correlation between unit cellulose and stalk breaking strength. Stalk breaking strength was highly correlated with the amount of cellulose in a unit stalk length. (correlation coefficient, r: 0.76; 0.63; 0.92; 0.86.) While these correlations are specifically related to the different CesA genes, it should be noted that in general the same correlation would apply. In other words, it is expected this would apply to low cellulose levels as well as higher cellulose levels.

FIG. 4: Unrooted cladogram of CesA proteins from different species. Sequences are labeled by prefixes. This cladogram demonstrates the relevance of the maize genes to those of Arabidopsis and rice. Prefixes: At, Arabidopsis thaliana; Gh, Gossypium herbaceum; Lj, Lotus japonicus; Mt, Medicago truncatula; Na, Nicotiana alata; Os, Oriza sativa; Pc, Populus canescens; Ptr, Populus tremula×tremuloides; Ze, Zinnia elegans; Zm, Zea mays.

FIG. 5: Expression pattern of CesA10, 11, and 12 in different maize tissues. All three genes are nearly synchronously expressed in tissues rich in secondary wall.

FIG. 6: Effect of overexpression of different CesA genes on plant height in corn. Whereas the overexpression of CesA8 led to an increase in height, CesA4 and CesA5 had not effect. Overexpression of CesA1 resulted in stunted plants.

FIG. 7: Effect of the overexpression of different CesA genes on the amount of cellulose in a unit length of the stalk tissue below ear in corn. CesA4 and CesA8, when overexpressed, resulted in an increased cellulose/length, CesA5 had no effect, and CesA1 resulted in reduced cellulose/length.

FIG. 8: Unrooted single most parsimonious tree of the CESA proteins from maize and Arabidopsis found by Branch and Bound algorithm of PAUP program. Branch lengths are proportionate to the inferred number of amino acid substitutions, which are shown in bold font. Bootstrap values (%) supporting the monophyletic groups are shown along the branches in parentheses.

Deduced protein sequences for the Arabidopsis CESA proteins were downloaded from the web site: cellwall.stanford.edu. Maize sequences for the genes CesA1-9 are available in GenBank (Holland et al., 2000). Sequence alignment was carried out using CLUSTAL W program (Thompson et al., 1994). Parsimony and neighbor-joining analyses were performed using the PAUP program (Swofford, 1998). To assess the degree of support for each branch on the tree, bootstrap analysis with 500 replicates was performed (Felsenstein, 1985). Maximum-likelihood tree was also reconstructed using proML algorithm implemented in the PHYLIP package by J. Felsenstein (Phylogeny Inference Package, version 3.6a2.1; web site: evolution.genetics.washington.edu/phylip.html). Both neighbor-joining and maximum-likelihood trees showed very similar tree topologies as that of the maximally parsimonious tree with minor terminal branch differences.

FIG. 9: Expression of the maize CesA genes in different tissues as compiled from the MPSS database. The data are averaged over 76 different libraries. The number of libraries for each tissue was: root, 12; leaf, 13; stalk, 6; ear, 10; silk, 7; kernel, 2; embryo, 10; endosperm, 13; and pericarp, 3. The average for the total number of tags across the 79 libraries was 1,370,525 with a range of 1,223, 721 for a stalk library to 2,154,139 for a root library. The average for the adjusted number of unique tags was 45,293 with a range of 15,226 in an endosperm library to 87,030 for a root library.

FIG. 10: Expression of the maize CesA genes in different tissues compiled from the MPSS database. Total and adjusted unique tags numbered as follows, respectively: elongation zone, 1,351,429 and 25,850; transition zone, 1,324,473 and 32,425; and vascular bundles, 1,338,456 and 29,329. Inset, cellulose concentration in different tissues used for MPSS analysis.

FIG. 11: Mechanical strength differences between bk2 and its wildtype sib. Internodal flexural strength of the brittle stalk (bk2) mutant and its wildtype sib were measured below the ear one week after flowering. The seeds from the same selfed ear segregating for bk2 were grown in the greenhouse in pots. These data are for the third internode below the ear node obtained one week after flowering.

### DETAILED DESCRIPTION OF THE INVENTION

### Overview

A. Nucleic Acids and Protein of the Present Invention

Unless otherwise stated, the polynucleotide and polypeptide sequences identified in Table 1 represent polynucleotides and polypeptides of the present invention. Table 1 cross-references these polynucleotide and polypeptides to their gene name and internal database identification number (SEQ ID NO.). A nucleic acid of the present invention comprises a polynucleotide of the present invention. A protein of the present invention comprises a polypeptide of the present invention.

TABLE 1

| | Database ID | Polynucleotide | Polypeptide |
|---|---|---|---|
| Gene Name | NO: | SEQ ID NO: | SEQ ID NO: |
| Cellulose synthase | CesA-1 | 1 | 2 |
| Cellulose synthase | CesA-2 | 45 | 46 |
| Cellulose synthase | CesA-3 | 5 | 6 |
| Cellulose synthase | CesA-4 | 9 | 10 |
| Cellulose synthase | CesA-5 | 13 | 14 |
| Cellulose synthase | CesA-6 | 41 | 42 |
| Cellulose synthase | CesA-7 | 49 | 50 |
| Cellulose synthase | CesA-8 | 17 | 18 |
| Cellulose synthase | CesA-9 | 21 | 22 |
| Cellulose synthase | CesA-10 | 25 | 26 |
| Cellulose synthase | CesA-11 | 27 | 28 |
| Cellulose synthase | CesA-12 | 29 | 30 |

Table 2 further provides a comparison detailing the homology as a percentage of the 12 CesA genes from maize that have been described herein (see also “Related Applications” above).

TABLE 2

| | CesA1 | CesA2 | CesA3 | CesA4 | CesA5 | CesA6 | CesA7 | CesA8 | CesA9 | CesA10 | CesA11 | CesA12 |
|---|---|---|---|---|---|---|---|---|---|---|---|---|
| CesA1 | | 93 | 60 | 59 | 60 | 55 | 55 | 57 | 61 | 51 | 51 | 46 |
| CesA2 | | | 60 | 59 | 61 | 55 | 55 | 57 | 61 | 51 | 51 | 47 |
| CesA3 | | | | 47 | 48 | 49 | 45 | 46 | 49 | 46 | 52 | 50 |
| CesA4 | | | | | 77 | 54 | 52 | 58 | 86 | 54 | 53 | 52 |
| CesA5 | | | | | | 55 | 53 | 57 | 75 | 52 | 52 | 51 |
| CesA6 | | | | | | | 74 | 73 | 56 | 56 | 55 | 53 |
| CesA7 | | | | | | | | 70 | 54 | 50 | 48 | 46 |
| CesA8 | | | | | | | | | 59 | 55 | 52 | 51 |
| CesA9 | | | | | | | | | | 52 | 52 | 50 |
| CesA10 | | | | | | | | | | | 53 | 64 |
| CesA11 | | | | | | | | | | | | 56 |
| CesA12 | | | | | | | | | | | | |

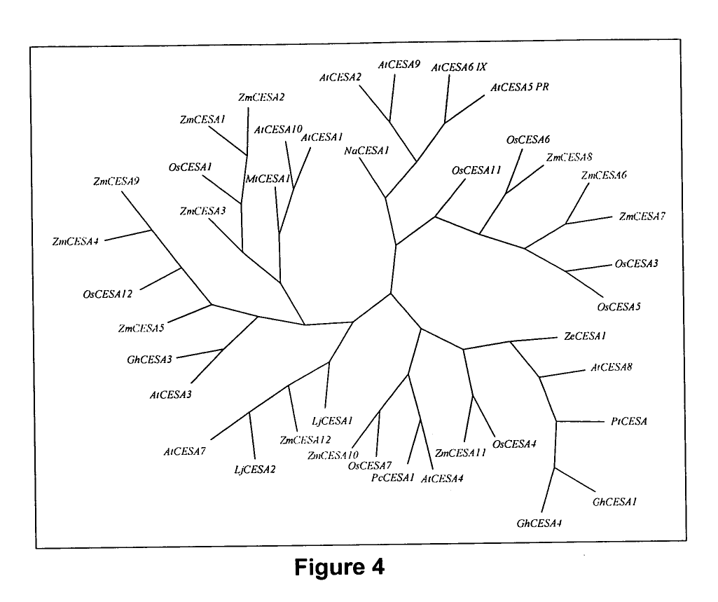

Further characterization of the CesA group is provided in FIG. 4, as a consensus tree for plant Ces A proteins. It describes the relationship between Ces A from maize, rice and Arabidopsis sources.

B. Exemplary Utility of the Present Invention

The present invention provides utility in such exemplary applications as improvement of stalk quality for improved stand lodging or standability or silage digestibility. Further, the present invention provides for an increased concentration of cellulose in the pericarp, hardening the kernel and thus improving its handling ability. Stalk lodging at maturity can cause significant yield losses in corn. Environmental stresses from flowering to harvest, such as drought and nutrient deficiency, further worsen this problem. The effect of abiotic stresses is exacerbated by biotic factors, such as stalk rot resulting from the soil-living pathogens growing through the ground tissue.

Maize hybrids known to be resistant to stalk lodging have mechanically stronger stalks. At the compositional level, cellulose in a unit stalk length is highly correlated with breaking strength. The present invention provides for modulation of cellulose synthase composition leading to increased stalk strength.

### Definitions

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole. Section headings provided throughout the specification are not limitations to the various objects and embodiments of the present invention.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17: 477-498 (1989)). Thus, the maize preferred codon for a particular amino acid may be derived from known gene sequences from maize. Maize codon usage for 28 genes from maize plants is listed in Table 4 of Murray et al., supra.

As used herein “full-length sequence” in reference to a specified polynucleotide or its encoded protein means having the entire amino acid sequence of a native (non-synthetic), endogenous, biologically (e.g., structurally or catalytically) active form of the specified protein. Methods to determine whether a sequence is full-length are well known in the art, including such exemplary techniques as northern or western blots, primer extension, S1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid is a nucleic acid that originates from a foreign species, or, if from the same species, is substantially modified from its native form in composition and/or genomic locus by human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The term “introduced” includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection”, “transformation” and “transduction”.

The term “isolated” refers to material, such as a nucleic acid or a protein, which is: (1) substantially or essentially free from components which normally accompany or interact with it as found in its natural environment. The isolated material optionally comprises material not found with the material in its natural environment; or (2) if the material is in its natural environment, the material has been synthetically altered or synthetically produced by deliberate human intervention and/or placed at a different location within the cell. The synthetic alteration or creation of the material can be performed on the material within or apart from its natural state. For example, a naturally-occurring nucleic acid becomes an isolated nucleic acid if it is altered or produced by non-natural, synthetic methods, or if it is transcribed from DNA which has been altered or produced by non-natural, synthetic methods. The isolated nucleic acid may also be produced by the synthetic re-arrangement (“shuffling”) of a part or parts of one or more allelic forms of the gene of interest. Likewise, a naturally-occurring nucleic acid (e.g., a promoter) becomes isolated if it is introduced to a different locus of the genome. Nucleic acids which are “isolated,” as defined herein, are also referred to as “heterologous” nucleic acids. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells, Zarling et al., WO 93/22443 (PCT/US93/03868).

As used herein, “nucleic acid” includes reference to a deoxyribonucleotide or ribonucleotide polymer, or chimeras thereof, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism, tissue, or of a cell type from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2^{nd} ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant parts or organs (e.g., leaves, stems, roots, etc.), plant cells, seeds and progeny of same. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. A particularly preferred plant is Zea mays.

As used herein, “polynucleotide” includes reference to a deoxyribopolynucleotide, ribopolynucleotide, or chimeras or analogs thereof that have the essential nature of a natural deoxy- or ribo-nucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of human intervention. The term “recombinant” as used herein does not encompass the alteration of the cell or vector by naturally occurring events (e.g., spontaneous mutation, natural transformation/transduction/transposition) such as those occurring without human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity, and most preferably 100% sequence identity (i.e., complementary) with each other.

The term “stringent conditions” or “stringent hybridization conditions” includes reference to conditions under which a probe will selectively hybridize to its target sequence, to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (“Tₘ”) for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. Hybridization and/or wash conditions can be applied for at least 10, 30, 60, 90, 120, or 240 minutes. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995).

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in introduction of a polynucleotide of the present invention into a host cell. Vectors are often replicons. Expression vectors permit transcription of a nucleic acid inserted therein.

The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al, J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

Unless otherwise stated, nucleotide and protein identity/similarity values provided herein are calculated using GAP (GCG Version 10) under default values.

GAP (Global Alignment Program) can also be used to compare a polynucleotide or polypeptide of the present invention with a reference sequence. GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 0, 1, 2, 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff (1989) Proc. Natl. Acad. Sci. USA 89:10915).

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

### Utilities

The present invention provides, among other things, compositions and methods for modulating (i.e., increasing or decreasing) the level of polynucleotides and polypeptides of the present invention in plants. In particular, the polynucleotides and polypeptides of the present invention can be expressed temporally or spatially, e.g., at developmental stages, in tissues, and/or in quantities, which are uncharacteristic of non-recombinantly engineered plants.

The present invention also provides isolated nucleic acids comprising polynucleotides of sufficient length and complementarity to a polynucleotide of the present invention to use as probes or amplification primers in the detection, quantitation, or isolation of gene transcripts. For example, isolated nucleic acids of the present invention can be used as probes in detecting deficiencies in the level of mRNA in screenings for desired transgenic plants, for detecting mutations in the gene (e.g., substitutions, deletions, or additions), for monitoring upregulation of expression or changes in enzyme activity in screening assays of compounds, for detection of any number of allelic variants (polymorphisms), orthologs, or paralogs of the gene, or for site directed mutagenesis in eukaryotic cells (see, e.g., U.S. Pat. No. 5,565,350). The isolated nucleic acids of the present invention can also be used for recombinant expression of their encoded polypeptides, or for use as immunogens in the preparation and/or screening of antibodies. The isolated nucleic acids of the present invention can also be employed for use in sense or antisense suppression of one or more genes of the present invention in a host cell, tissue, or plant. Attachment of chemical agents which bind, intercalate, cleave and/or crosslink to the isolated nucleic acids of the present invention can also be used to modulate transcription or translation.

The present invention also provides isolated proteins comprising a polypeptide of the present invention (e.g., preproenzyme, proenzyme, or enzymes). The present invention also provides proteins comprising at least one epitope from a polypeptide of the present invention. The proteins of the present invention can be employed in assays for enzyme agonists or antagonists of enzyme function, or for use as immunogens or antigens to obtain antibodies specifically immunoreactive with a protein of the present invention. Such antibodies can be used in assays for expression levels, for identifying and/or isolating nucleic acids of the present invention from expression libraries, for identification of homologous polypeptides from other species, or for purification of polypeptides of the present invention.

The isolated nucleic acids and polypeptides of the present invention can be used over a broad range of plant types, particularly monocots such as the species of the family Gramineae including Hordeum, Secale, Oryza, Triticum, Sorghum (e.g., S. bicolor) and Zea (e.g., Z. mays), and dicots such as Glycine.

The isolated nucleic acid and proteins of the present invention can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browallia, Pisum, Phaseolus, Lolium, and Avena.

### Nucleic Acids

The present invention provides, among other things, isolated nucleic acids of RNA, DNA, and analogs and/or chimeras thereof, comprising a polynucleotide of the present invention.

A polynucleotide of the present invention is inclusive of those in Table 1 and:

(a) an isolated polynucleotide encoding a polypeptide of the present invention such as those referenced in Table 1, including exemplary polynucleotides of the present invention;

(b) an isolated polynucleotide which is the product of amplification from a plant nucleic acid library using primer pairs which selectively hybridize under stringent conditions to loci within a polynucleotide of the present invention;

(c) an isolated polynucleotide which selectively hybridizes to a polynucleotide of (a) or (b);

(d) an isolated polynucleotide having a specified sequence identity with polynucleotides of (a), (b), or (c);

(e) an isolated polynucleotide encoding a protein having a specified number of contiguous amino acids from a prototype polypeptide, wherein the protein is specifically recognized by antisera elicited by presentation of the protein and wherein the protein does not detectably immunoreact to antisera which has been fully immunosorbed with the protein;

(f) complementary sequences of polynucleotides of (a), (b), (c), (d), or (e); and

(g) an isolated polynucleotide comprising at least a specific number of contiguous nucleotides from a polynucleotide of (a), (b), (c), (d), (e), or (f);

(h) an isolated polynucleotide from a full-length enriched cDNA library having the physico-chemical property of selectively hybridizing to a polynucleotide of (a), (b), (c), (d), (e), (f), or (g);

(i) an isolated polynucleotide made by the process of: 1) providing a full-length enriched nucleic acid library, 2) selectively hybridizing the polynucleotide to a polynucleotide of (a), (b), (c), (d), (e), (f), (g), or (h), thereby isolating the polynucleotide from the nucleic acid library.

A. Polynucleotides Encoding A Polypeptide of the Present Invention

As indicated in (a), above, the present invention provides isolated nucleic acids comprising a polynucleotide of the present invention, wherein the polynucleotide encodes a polypeptide of the present invention. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG , which is ordinarily the only codon for tryptophan) can be modified to yield a functionally identical molecule. Thus, each silent variation of a nucleic acid which encodes a polypeptide of the present invention is implicit in each described polypeptide sequence and is within the scope of the present invention. Accordingly, the present invention includes polynucleotides of the present invention and polynucleotides encoding a polypeptide of the present invention.

B. Polynucleotides Amplified from a Plant Nucleic Acid Library

As indicated in (b), above, the present invention provides an isolated nucleic acid comprising a polynucleotide of the present invention, wherein the polynucleotides are amplified, under nucleic acid amplification conditions, from a plant nucleic acid library. Nucleic acid amplification conditions for each of the variety of amplification methods are well known to those of ordinary skill in the art. The plant nucleic acid library can be constructed from a monocot such as a cereal crop. Exemplary cereals include maize, sorghum, alfalfa, canola, wheat, or rice. The plant nucleic acid library can also be constructed from a dicot such as soybean. Zea mays lines B73, PHRE1, A632, BMS-P2 #10, W23, and Mo17 are known and publicly available. Other publicly known and available maize lines can be obtained from the Maize Genetics Cooperation (Urbana, Ill.). Wheat lines are available from the Wheat Genetics Resource Center (Manhattan, Kans.).

The nucleic acid library may be a cDNA library, a genomic library, or a library generally constructed from nuclear transcripts at any stage of intron processing. cDNA libraries can be normalized to increase the representation of relatively rare cDNAs. In optional embodiments, the cDNA library is constructed using an enriched full-length cDNA synthesis method. Examples of such methods include Oligo-Capping (Maruyama, K. and Sugano, S. Gene 138: 171-174, 1994), Biotinylated CAP Trapper (Carninci, et al. Genomics 37: 327-336, 1996), and CAP Retention Procedure (Edery, E., Chu, L. L., et al. Molecular and Cellular Biology 15: 3363-3371, 1995). Rapidly growing tissues or rapidly dividing cells are preferred for use as an mRNA source for construction of a cDNA library. Growth stages of maize are described in “How a Corn Plant Develops,” Special Report No. 48, Iowa State University of Science and Technology Cooperative Extension Service, Ames, Iowa, Reprinted February 1993.

A polynucleotide of this embodiment (or subsequences thereof) can be obtained, for example, by using amplification primers which are selectively hybridized and primer extended, under nucleic acid amplification conditions, to at least two sites within a polynucleotide of the present invention, or to two sites within the nucleic acid which flank and comprise a polynucleotide of the present invention, or to a site within a polynucleotide of the present invention and a site within the nucleic acid which comprises it. Methods for obtaining 5′ and/or 3′ ends of a vector insert are well known in the art. See, e.g., RACE (Rapid Amplification of Complementary Ends) as described in Frohman, M. A., in PCR Protocols: A Guide to Methods and Applications, M. A. Innis, D. H. Gelfand, J. J. Sninsky, T. J. White, Eds. (Academic Press, Inc., San Diego), pp. 28-38 (1990)); see also, U.S. Pat. No. 5,470,722, and Current Protocols in Molecular Biology, Unit 15.6, Ausubel, et al., Eds., Greene Publishing and Wiley-Interscience, New York (1995); Frohman and Martin, Techniques 1:165 (1989).

Optionally, the primers are complementary to a subsequence of the target nucleic acid which they amplify but may have a sequence identity ranging from about 85% to 99% relative to the polynucleotide sequence which they are designed to anneal to. As those skilled in the art will appreciate, the sites to which the primer pairs will selectively hybridize are chosen such that a single contiguous nucleic acid can be formed under the desired nucleic acid amplification conditions. The primer length in nucleotides is selected from the group of integers consisting of from at least 15 to 50. Thus, the primers can be at least 15, 18, 20, 25, 30, 40, or 50 nucleotides in length. Those of skill will recognize that a lengthened primer sequence can be employed to increase specificity of binding (i.e., annealing) to a target sequence. A non-annealing sequence at the 5′ end of a primer (a “tail”) can be added, for example, to introduce a cloning site at the terminal ends of the amplicon.

The amplification products can be translated using expression systems well known to those of skill in the art. The resulting translation products can be confirmed as polypeptides of the present invention by, for example, assaying for the appropriate catalytic activity (e.g., specific activity and/or substrate specificity), or verifying the presence of one or more epitopes which are specific to a polypeptide of the present invention. Methods for protein synthesis from PCR derived templates are known in the art and available commercially. See, e.g., Amersham Life Sciences, Inc, Catalog '97, p.354.

C. Polynucleotides which Selectively Hybridize to a Polynucleotide of (A) or (B)

As indicated in (c), above, the present invention provides isolated nucleic acids comprising polynucleotides of the present invention, wherein the polynucleotides selectively hybridize, under selective hybridization conditions, to a polynucleotide of sections (A) or (B) as discussed above. Thus, the polynucleotides of this embodiment can be used for isolating, detecting, and/or quantifying nucleic acids comprising the polynucleotides of (A) or (B). For example, polynucleotides of the present invention can be used to identify, isolate, or amplify partial or full-length clones in a deposited library. In some embodiments, the polynucleotides are genomic or cDNA sequences isolated or otherwise complementary to a cDNA from a dicot or monocot nucleic acid library. Exemplary species of monocots and dicots include, but are not limited to: maize, canola, soybean, cotton, wheat, sorghum, sunflower, alfalfa, oats, sugar cane, millet, barley, and rice. The cDNA library comprises at least 50% to 95% full-length sequences (for example, at least 50%, 60%, 70%, 80%, 90%, or 95% full-length sequences). The cDNA libraries can be normalized to increase the representation of rare sequences. See, e.g., U.S. Pat. No. 5,482,845. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% to 80% sequence identity and can be employed to identify orthologous or paralogous sequences.

D. Polynucleotides Having a Specific Sequence Identity with the Polynucleotides of (A), (B) or (C)

As indicated in (d), above, the present invention provides isolated nucleic acids comprising polynucleotides of the present invention, wherein the polynucleotides have a specified identity at the nucleotide level to a polynucleotide as disclosed above in sections (A), (B), or (C), above. Identity can be calculated using, for example, the BLAST, CLUSTALW, or GAP algorithms under default conditions. The percentage of identity to a reference sequence is at least 50% and, rounded upwards to the nearest integer, can be expressed as an integer selected from the group of integers consisting of from 50 to 99. Thus, for example, the percentage of identity to a reference sequence can be at least 60%, 70%, 75%, 80%, 85%, 90%, or 95%.

Optionally, the polynucleotides of this embodiment will encode a polypeptide that will share an epitope with a polypeptide encoded by the polynucleotides of sections (A), (B), or (C). Thus, these polynucleotides encode a first polypeptide which elicits production of antisera comprising antibodies which are specifically reactive to a second polypeptide encoded by a polynucleotide of (A), (B), or (C). However, the first polypeptide does not bind to antisera raised against itself when the antisera has been fully immunosorbed with the first polypeptide. Hence, the polynucleotides of this embodiment can be used to generate antibodies for use in, for example, the screening of expression libraries for nucleic acids comprising polynucleotides of (A), (B), or (C), or for purification of, or in immunoassays for, polypeptides encoded by the polynucleotides of (A), (B), or (C). The polynucleotides of this embodiment comprise nucleic acid sequences which can be employed for selective hybridization to a polynucleotide encoding a polypeptide of the present invention.

Screening polypeptides for specific binding to antisera can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT patent publication Nos. 91/17271, 91/18980, 91/19818, and 93/08278. Other systems for generating libraries of peptides have aspects of both in vitro chemical synthesis and recombinant methods. See, PCT Patent publication Nos. 92/05258, 92/14843, and 97/20078. See also, U.S. Pat Nos. 5,658,754; and 5,643,768. Peptide display libraries, vectors, and screening kits are commercially available from such suppliers as Invitrogen (Carlsbad, Calif.).

E. Polynucleotides Encoding a Protein Having a Subsequence from a Prototype Polypeptide and Cross-Reactive to the Prototype Polypeptide

As indicated in (e), above, the present invention provides isolated nucleic acids comprising polynucleotides of the present invention, wherein the polynucleotides encode a protein having a subsequence of contiguous amino acids from a prototype polypeptide of the present invention such as are provided in (a), above. The length of contiguous amino acids from the prototype polypeptide is selected from the group of integers consisting of from at least 10 to the number of amino acids within the prototype sequence. Thus, for example, the polynucleotide can encode a polypeptide having a subsequence having at least 10, 15, 20, 25, 30, 35, 40, 45, or 50, contiguous amino acids from the prototype polypeptide. Further, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

The proteins encoded by polynucleotides of this embodiment, when presented as an immunogen, elicit the production of polyclonal antibodies which specifically bind to a prototype polypeptide such as but not limited to, a polypeptide encoded by the polynucleotide of (a) or (b), above. Generally, however, a protein encoded by a polynucleotide of this embodiment does not bind to antisera raised against the prototype polypeptide when the antisera has been fully immunosorbed with the prototype polypeptide. Methods of making and assaying for antibody binding specificity/affinity are well known in the art. Exemplary immunoassay formats include ELISA, competitive immunoassays, radioimmunoassays, Western blots, indirect immunofluorescent assays and the like.

In a preferred assay method, fully immunosorbed and pooled antisera which is elicited to the prototype polypeptide can be used in a competitive binding assay to test the protein. The concentration of the prototype polypeptide required to inhibit 50% of the binding of the antisera to the prototype polypeptide is determined. If the amount of the protein required to inhibit binding is less than twice the amount of the prototype protein, then the protein is said to specifically bind to the antisera elicited to the immunogen. Accordingly, the proteins of the present invention embrace allelic variants, conservatively modified variants, and minor recombinant modifications to a prototype polypeptide.

A polynucleotide of the present invention optionally encodes a protein having a molecular weight as the non-glycosylated protein within 20% of the molecular weight of the full-length non-glycosylated polypeptides of the present invention. Molecular weight can be readily determined by SDS-PAGE under reducing conditions. Optionally, the molecular weight is within 15% of a full length polypeptide of the present invention, more preferably within 10% or 5%, and most preferably within 3%, 2%, or 1% of a full length polypeptide of the present invention.

Optionally, the polynucleotides of this embodiment will encode a protein having a specific enzymatic activity at least 50%, 60%, 80%, or 90% of a cellular extract comprising the native, endogenous full-length polypeptide of the present invention. Further, the proteins encoded by polynucleotides of this embodiment will optionally have a substantially similar affinity constant (Kₘ) and/or catalytic activity (i.e., the microscopic rate constant, k_{cat}) as the native endogenous, full-length protein. Those of skill in the art will recognize that k_{cat}/Kₘ value determines the specificity for competing substrates and is often referred to as the specificity constant. Proteins of this embodiment can have a k_{cat}/Kₘ value at least 10% of a full-length polypeptide of the present invention as determined using the endogenous substrate of that polypeptide. Optionally, the k_{cat}/Kₘ value will be at least 20%, 30%, 40%, 50%, and most preferably at least 60%, 70%, 80%, 90%, or 95% the k_{cat}/Kₘ value of the full-length polypeptide of the present invention. Determination of k_{cat}, Kₘ, and k_{cat}/Kₘ can be determined by any number of means well known to those of skill in the art. For example, the initial rates (i.e., the first 5% or less of the reaction) can be determined using rapid mixing and sampling techniques (e.g., continuous-flow, stopped-flow, or rapid quenching techniques), flash photolysis, or relaxation methods (e.g., temperature jumps) in conjunction with such exemplary methods of measuring as spectrophotometry, spectrofluorimetry, nuclear magnetic resonance, or radioactive procedures. Kinetic values are conveniently obtained using a Lineweaver-Burk or Eadie-Hofstee plot.

F. Polynucleotides Complementary to the Polynucleotides of (A)-(E)

As indicated in (f), above, the present invention provides isolated nucleic acids comprising polynucleotides complementary to the polynucleotides of paragraphs A-E, above. As those of skill in the art will recognize, complementary sequences base-pair throughout the entirety of their length with the polynucleotides of sections (A)-(E) (i.e., have 100% sequence identity over their entire length). Complementary bases associate through hydrogen bonding in double stranded nucleic acids. For example, the following base pairs are complementary: guanine and cytosine; adenine and thymine; and adenine and uracil.

G. Polynucleotides which are Subsequences of the Polynucleotides of (A)-(F)

As indicated in (g), above, the present invention provides isolated nucleic acids comprising polynucleotides which comprise at least 15 contiguous bases from the polynucleotides of sections (A) through (F) as discussed above. The length of the polynucleotide is given as an integer selected from the group consisting of from at least 15 to the length of the nucleic acid sequence from which the polynucleotide is a subsequence of. Thus, for example, polynucleotides of the present invention are inclusive of polynucleotides comprising at least 15, 20, 25, 30, 40, 50, 60, 75, or 100 contiguous nucleotides in length from the polynucleotides of (A)-(F). Optionally, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

Subsequences can be made by in vitro synthetic, in vitro biosynthetic, or in vivo recombinant methods. In optional embodiments, subsequences can be made by nucleic acid amplification. For example, nucleic acid primers will be constructed to selectively hybridize to a sequence (or its complement) within, or co-extensive with, the coding region.

The subsequences of the present invention can comprise structural characteristics of the sequence from which it is derived. Alternatively, the subsequences can lack certain structural characteristics of the larger sequence from which it is derived such as a poly (A) tail. Optionally, a subsequence from a polynucleotide encoding a polypeptide having at least one epitope in common with a prototype polypeptide sequence as provided in (a), above, may encode an epitope in common with the prototype sequence. Alternatively, the subsequence may not encode an epitope in common with the prototype sequence but can be used to isolate the larger sequence by, for example, nucleic acid hybridization with the sequence from which it's derived. Subsequences can be used to modulate or detect gene expression by introducing into the subsequences compounds which bind, intercalate, cleave and/or crosslink to nucleic acids. Exemplary compounds include acridine, psoralen, phenanthroline, naphthoquinone, daunomycin or chloroethylaminoaryl conjugates.

H. Polynucleotides from a Full-Length Enriched cDNA Library Having the Physico-Chemical Property of Selectively Hybridizing to a Polynucleotide of (A)-(G)

As indicated in (h), above, the present invention provides an isolated polynucleotide from a full-length enriched cDNA library having the physico-chemical property of selectively hybridizing to a polynucleotide of paragraphs (A), (B), (C), (D), (E), (F), or (G) as discussed above. Methods of constructing full-length enriched cDNA libraries are known in the art and discussed briefly below. The cDNA library comprises at least 50% to 95% full-length sequences (for example, at least 50%, 60%, 70%, 80%, 90%, or 95% full-length sequences). The cDNA library can be constructed from a variety of tissues from a monocot or dicot at a variety of developmental stages. Exemplary species include maize, wheat, rice, canola, soybean, cotton, sorghum, sunflower, alfalfa, oats, sugar cane, millet, barley, and rice. Methods of selectively hybridizing, under selective hybridization conditions, a polynucleotide from a full-length enriched library to a polynucleotide of the present invention are known to those of ordinary skill in the art. Any number of stringency conditions can be employed to allow for selective hybridization. In optional embodiments, the stringency allows for selective hybridization of sequences having at least 70%, 75%, 80%, 85%, 90%, 95%, or 98% sequence identity over the length of the hybridized region. Full-length enriched cDNA libraries can be normalized to increase the representation of rare sequences.

I. Polynucleotide Products Made by a cDNA Isolation Process

As indicated in (I), above, the present invention provides an isolated polynucleotide made by the process of: 1) providing a full-length enriched nucleic acid library, 2) selectively hybridizing the polynucleotide to a polynucleotide of paragraphs (A), (B), (C), (D), (E), (F), (G, or (H) as discussed above, and thereby isolating the polynucleotide from the nucleic acid library. Full-length enriched nucleic acid libraries are constructed as discussed in paragraph (G) and below. Selective hybridization conditions are as discussed in paragraph (G). Nucleic acid purification procedures are well known in the art. Purification can be conveniently accomplished using solid-phase methods; such methods are well known to those of skill in the art and kits are available from commercial suppliers such as Advanced Biotechnologies (Surrey, U K). For example, a polynucleotide of paragraphs (A)-(H) can be immobilized to a solid support such as a membrane, bead, or particle. See, e.g., U.S. Pat. No. 5,667,976. The polynucleotide product of the present process is selectively hybridized to an immobilized polynucleotide and the solid support is subsequently isolated from non-hybridized polynucleotides by methods including, but not limited to, centrifugation, magnetic separation, filtration, electrophoresis, and the like.

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a monocot such as maize, rice, or wheat, or a dicot such as soybean.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. A polynucleotide of the present invention can be attached to a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known and extensively described in the art. For a description of various nucleic acids see, for example, Stratagene Cloning Systems, Catalogs 1999 (La Jolla, Calif.); and, Amersham Life Sciences, Inc, Catalog '99 (Arlington Heights, Ill.).

A. Recombinant Methods for Constructing Nucleic Acids

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes which selectively hybridize, under stringent conditions, to the polynucleotides of the present invention are used to identify the desired sequence in a cDNA or genomic DNA library. Isolation of RNA, and construction of cDNA and genomic libraries is well known to those of ordinary skill in the art. See, e.g., Plant Molecular Biology: A Laboratory Manual, Clark, Ed., Springer-Verlag, Berlin (1997); and, Current Protocols in Molecular Biology, Ausubel, et al., Eds., Greene Publishing and Wiley-Interscience, New York (1995).

A1. Full-Length Enriched cDNA Libraries

A number of cDNA synthesis protocols have been described which provide enriched full-length cDNA libraries. Enriched full-length cDNA libraries are constructed to comprise at least 600%, and more preferably at least 70%, 80%, 90% or 95% full-length inserts amongst clones containing inserts. The length of insert in such libraries can be at least 2, 3, 4, 5, 6, 7, 8, 9, 10 or more kilobase pairs. Vectors to accommodate inserts of these sizes are known in the art and available commercially. See, e.g., Stratagene's lambda ZAP Express (cDNA cloning vector with 0 to 12 kb cloning capacity). An exemplary method of constructing a greater than 95% pure full-length cDNA library is described by Carninci et al., Genomics, 37:327-336 (1996). Other methods for producing full-length libraries are known in the art. See, e.g., Edery et al., Mol. Cell Biol., 15(6):3363-3371 (1995); and, PCT Application WO 96/34981.

A2 Normalized or Subtracted cDNA Libraries

A non-normalized cDNA library represents the mRNA population of the tissue it was made from. Since unique clones are out-numbered by clones derived from highly expressed genes their isolation can be laborious. Normalization of a cDNA library is the process of creating a library in which each clone is more equally represented. Construction of normalized libraries is described in Ko, Nucl. Acids. Res., 18(19):5705-5711 (1990); Patanjali et al., Proc. Natl. Acad. U.S.A., 88:1943-1947 (1991); U.S. Pat. Nos. 5,482,685, 5,482,845, and 5,637,685. In an exemplary method described by Soares et al., normalization resulted in reduction of the abundance of clones from a range of four orders of magnitude to a narrow range of only 1 order of magnitude. Proc. Natl. Acad. Sci. USA, 91:9228-9232 (1994).

Subtracted cDNA libraries are another means to increase the proportion of less abundant cDNA species. In this procedure, cDNA prepared from one pool of mRNA is depleted of sequences present in a second pool of mRNA by hybridization. The cDNA:mRNA hybrids are removed and the remaining un-hybridized cDNA pool is enriched for sequences unique to that pool. See, Foote et al. in, Plant Molecular Biology: A Laboratory Manual, Clark, Ed., Springer-Verlag, Berlin (1997); Kho and Zarbl, Technique, 3(2):58-63 (1991); Sive and St. John, Nucl. Acids Res., 16(22):10937 (1988); Current Protocols in Molecular Biology, Ausubel, et al., Eds., Greene Publishing and Wiley-Interscience, New York (1995); and, Swaroop et al., Nucl. Acids Res., 19)8):1954 (1991). cDNA subtraction kits are commercially available. See, e.g., PCR-Select (Clontech, Palo Alto, Calif.).

To construct genomic libraries, large segments of genomic DNA are generated by fragmentation, e.g. using restriction endonucleases, and are ligated with vector DNA to form concatemers that can be packaged into the appropriate vector. Methodologies to accomplish these ends, and sequencing methods to verify the sequence of nucleic acids are well known in the art. Examples of appropriate molecular biological techniques and instructions sufficient to direct persons of skill through many construction, cloning, and screening methodologies are found in Sambrook, et al., Molecular Clonin^{g}: A Laboratory Manual, 2nd Ed., Cold Spring Harbor Laboratory Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel, et al., Eds., Greene Publishing and Wiley-Interscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a polynucleotide of the present invention such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent.

The nucleic acids of interest can also be amplified from nucleic acid samples using amplification techniques. For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. The T4 gene 32 protein (Boehringer Mannheim) can be used to improve yield of long PCR products.

PCR-based screening methods have been described. Wilfinger et al. describe a PCR-based method in which the longest cDNA is identified in the first step so that incomplete clones can be eliminated from study. BioTechniques, 22(3): 481-486 (1997). Such methods are particularly effective in combination with a full-length cDNA construction methodology, above.

B. Synthetic Methods for Constructing Nucleic Acids

The isolated nucleic acids of the present invention can also be prepared by direct chemical synthesis by methods such as the phosphotriester method of Narang et al., Meth. Enzymol. 68: 90-99 (1979); the phosphodiester method of Brown et al., Meth. Enzymol. 68: 109-151 (1979); the diethylphosphoramidite method of Beaucage et al., Tetra. Lett. 22: 1859-1862 (1981); the solid phase phosphoramidite triester method described by Beaucage and Caruthers, Tetra. Letts. 22(20): 1859-1862 (1981), e.g., using an automated synthesizer, e.g., as described in Needham-VanDevanter et al., Nucleic Acids Res., 12: 6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is best employed for sequences of about 100 bases or less, longer sequences may be obtained by the ligation of shorter sequences.

### Recombinant Expression Cassettes

The present invention further provides recombinant expression cassettes comprising a nucleic acid of the present invention. A nucleic acid sequence coding for the desired polypeptide of the present invention, for example a cDNA or a genomic sequence encoding a full length polypeptide of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, and the GRP1-8 promoter.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter which is inducible by hypoxia or cold stress, the Hsp70 promoter which is inducible by heat stress, and the PPDK promoter which is inducible by light.

Examples of promoters under developmental control include promoters that initiate transcription only, or preferentially, in certain tissues, such as leaves, roots, fruit, seeds, or flowers. Exemplary promoters include the anther-specific promoter 5126 (U.S. Pat. Nos. 5,689,049 and 5,689,051), glb-1 promoter, and gamma-zein promoter. Also see, for example, U.S. patent applications 60/155,859, and 60/163,114. The operation of a promoter may also vary depending on its location in the genome. Thus, an inducible promoter may become fully or partially constitutive in certain locations.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter concentration and/or composition of the proteins of the present invention in a desired tissue. Thus, in some embodiments, the nucleic acid construct will comprise a promoter, functional in a plant cell, operably linked to a polynucleotide of the present invention. Promoters useful in these embodiments include the endogenous promoters driving expression of a polypeptide of the present invention.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo by mutation, deletion, and/or substitution (see, Kmiec, U.S. Pat. No. 5,565,350; Zarling et al., PCT/US93/03868), or isolated promoters can be introduced into a plant cell in the proper orientation and distance from a cognate gene of a polynucleotide of the present invention so as to control the expression of the gene. Gene expression can be modulated under conditions suitable for plant growth so as to alter the total concentration and/or alter the composition of the polypeptides of the present invention in plant cell. Thus, the present invention provides compositions, and methods for making, heterologous promoters and/or enhancers operably linked to a native, endogenous (i.e., non-heterologous) form of a polynucleotide of the present invention.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence of the partial coding sequence to increase the amount of the mature message that accumulates in the cytosol. Inclusionon of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:11831200 (1987). Such intron enhancement of gene expression is typically greatest when placed near the 5′ end of the transcription unit. Use of maize introns Adh1-S intron 1, 2, and 6, the Bronze-1 intron are known in the art. See generally, The Maize Handbook, Chapter 116, Freeling and Walbot, Eds., Springer, N.Y. (1994). The vector comprising the sequences from a polynucleotide of the present invention will typically comprise a marker gene which confers a selectable phenotype on plant cells. Typical vectors useful for expression of genes in higher plants are well known in the art and include vectors derived from the tumor-inducing (Ti) plasmid of Agrobacterium tumefaciens described by Rogers et al., Meth. in Enzymol., 153:253-277 (1987).

A polynucleotide of the present invention can be expressed in either sense or anti-sense orientation as desired. It will be appreciated that control of gene expression in either sense or anti-sense orientation can have a direct impact on the observable plant characteristics. Antisense technology can be conveniently used to inhibit gene expression in plants. To accomplish this, a nucleic acid segment from the desired gene is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy et al., Proc. Nat'l. Acad. Sci. (USA) 85: 8805-8809 (1988); and Hiatt et al., U.S. Pat. No. 4,801,340.

Another method of suppression is sense suppression (i.e., co-supression). Introduction of nucleic acid configured in the sense orientation has been shown to be an effective means by which to block the transcription of target genes. For an example of the use of this method to modulate expression of endogenous genes see, Napoli et al., The Plant Cell 2: 279-289 (1990) and U.S. Pat. No. 5,034,323.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al., Nature 334: 585-591 (1988).

A variety of cross-linking agents, alkylating agents and radical generating species as pendant groups on polynucleotides of the present invention can be used to bind, label, detect, and/or cleave nucleic acids. For example, Vlassov, V. V., et al., Nucleic Acids Res (1986) 14:4065-4076, describe covalent bonding of a single-stranded DNA fragment with alkylating derivatives of nucleotides complementary to target sequences. A report of similar work by the same group is that by Knorre, D. G., et al., Biochimie (1985) 67:785-789. Iverson and Dervan also showed sequence-specific cleavage of single-stranded DNA mediated by incorporation of a modified nucleotide which was capable of activating cleavage (J Am Chem Soc (1987) 109:1241-1243). Meyer, R. B., et al., J Am Chem Soc (1989) 111:8517-8519, effect covalent crosslinking to a target nucleotide using an alkylating agent complementary to the single-stranded target nucleotide sequence. A photoactivated crosslinking to single-stranded oligonucleotides mediated by psoralen was disclosed by Lee, B. L., et al., Biochemistry (1988) 27:3197-3203. Use of crosslinking in triple-helix forming probes was also disclosed by Home, et al., J Am Chem Soc (1990) 112:2435-2437. Use of N4, N4-ethanocytosine as an alkylating agent to crosslink to single-stranded oligonucleotides has also been described by Webb and Matteucci, J Am Chem Soc (1986) 108:2764-2765; Nucleic Acids Res (1986) 14:7661-7674; Feteritz et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and, 5,681941.

### Proteins

The isolated proteins of the present invention comprise a polypeptide having at least 10 amino acids from a polypeptide of the present invention (or conservative variants thereof) such as those encoded by any one of the polynucleotides of the present invention as discussed more fully above (e.g., Table 1). The proteins of the present invention or variants thereof can comprise any number of contiguous amino acid residues from a polypeptide of the present invention, wherein that number is selected from the group of integers consisting of from 10 to the number of residues in a full-length polypeptide of the present invention. Optionally, this subsequence of contiguous amino acids is at least 15, 20, 25, 30, 35, or 40 amino acids in length, often at least 50, 60, 70, 80, or 90 amino acids in length. Further, the number of such subsequences can be any integer selected from the group consisting of from 1 to 20, such as 2, 3, 4, or 5.

The present invention further provides a protein comprising a polypeptide having a specified sequence identity/similarity with a polypeptide of the present invention. The percentage of sequence identity/similarity is an integer selected from the group consisting of from 50 to 99. Exemplary sequence identity/similarity values include 55%, 60%, 65%, 70%, 75%, 80%, 85%, 90%, and 95%. Sequence identity can be determined using, for example, the GAP, CLUSTALW, or BLAST algorithms.

As those of skill will appreciate, the present invention includes, but is not limited to, catalytically active polypeptides of the present invention (i.e., enzymes). Catalytically active polypeptides have a specific activity of at least 20%, 30%, or 40%, and preferably at least 50%, 60%, or 70%, and most preferably at least 80%, 90%, or 95% that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least 30%, 40%, or 50%, that of the native (non-synthetic), endogenous polypeptide; and more preferably at least 60%, 70%, 80%, or 90%. Methods of assaying and quantifying measures of enzymatic activity and substrate specificity (k_{cat}/Kₘ), are well known to those of skill in the art.

Generally, the proteins of the present invention will, when presented as an immunogen, elicit production of an antibody specifically reactive to a polypeptide of the present invention. Further, the proteins of the present invention will not bind to antisera raised against a polypeptide of the present invention which has been fully immunosorbed with the same polypeptide. Immunoassays for determining binding are well known to those of skill in the art. A preferred immunoassay is a competitive immunoassay. Thus, the proteins of the present invention can be employed as immunogens for constructing antibodies immunoreactive to a protein of the present invention for such exemplary utilities as immunoassays or protein purification techniques.

### Expression of Proteins in Host Cells

Using the nucleic acids of the present invention, one may express a protein of the present invention in a recombinantly engineered cell such as bacteria, yeast, insect, mammalian, or preferably plant cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter to direct transcription, a ribosome binding site for translational initiation, and a transcription/translation terminator. One of skill would recognize that modifications can be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced.

### Synthesis of Proteins

The proteins of the present invention can be constructed using non-cellular synthetic methods. Solid phase synthesis of proteins of less than about 50 amino acids in length may be accomplished by attaching the C-terminal amino acid of the sequence to an insoluble support followed by sequential addition of the remaining amino acids in the sequence. Techniques for solid phase synthesis are described by Barany and Merrifield, Solid-Phase Peptide Synthesis, pp. 3-284 in The Peptides: Analysis, Synthesis, Biology. Vol. 2: Special Methods in Peptide Synthesis, Part A.; Merrifield, et al., J. Am. Chem. Soc. 85: 2149-2156 (1963), and Stewart et al., Solid Phase Peptide Synthesis, 2nd ed., Pierce Chem. Co., Rockford, III. (1984). Proteins of greater length may be synthesized by condensation of the amino and carboxy termini of shorter fragments. Methods of forming peptide bonds by activation of a carboxy terminal end (e.g., by the use of the coupling reagent N,N′-dicycylohexylcarbodiimide) are known to those of skill.

### Purification of Proteins

The proteins of the present invention may be purified by standard techniques well known to those of skill in the art. Recombinantly produced proteins of the present invention can be directly expressed or expressed as a fusion protein. The recombinant protein is purified by a combination of cell lysis (e.g., sonication, French press) and affinity chromatography. For fusion products, subsequent digestion of the fusion protein with an appropriate proteolytic enzyme releases the desired recombinant protein.

The proteins of this invention, recombinant or synthetic, may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: N.Y. (1982); Deutscher, Guide to Protein Purification, Academic Press (1990). For example, antibodies may be raised to the proteins as described herein. Purification from E. coli can be achieved following procedures described in U.S. Pat. No. 4,511,503. The protein may then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

### Introduction of Nucleic Acids into Host Cells

The method of introducing a nucleic acid of the present invention into a host cell is not critical to the instant invention. Transformation or transfection methods are conveniently used. Accordingly, a wide variety of methods have been developed to insert a DNA sequence into the genome of a host cell to obtain the transcription and/or translation of the sequence to effect phenotypic changes in the organism. Thus, any method which provides for effective introduction of a nucleic acid may be employed.

A. Plant Transformation

A nucleic acid comprising a polynucleotide of the present invention is optionally introduced into a plant. Generally, the polynucleotide will first be incorporated into a recombinant expression cassette or vector. Isolated nucleic acid acids of the present invention can be introduced into plants according to techniques known in the art. Techniques for transforming a wide variety of higher plant species are well known and described in the technical, scientific, and patent literature. See, for example, Weising et al., Ann. Rev. Genet. 22: 421-477 (1988). For example, the DNA construct may be introduced directly into the genomic DNA of the plant cell using techniques such as electroporation, polyethylene glycol (PEG) poration, particle bombardment, silicon fiber delivery, or microinjection of plant cell protoplasts or embryogenic callus. See, e.g., Tomes, et al., Direct DNA Transfer into Intact Plant Cells Via Microprojectile Bombardment. pp.197-213 in Plant Cell, Tissue and Organ Culture, Fundamental Methods. eds. O. L. Gamborg and G. C. Phillips. Springer-Verlag Berlin Heidelberg N.Y., 1995; see, U.S. Pat. No. 5,990,387. The introduction of DNA constructs using PEG precipitation is described in Paszkowski et al., Embo J. 3: 2717-2722 (1984). Electroporation techniques are described in Fromm et al., Proc. Natl. Acad. Sci. (USA) 82: 5824 (1985). Ballistic transformation techniques are described in Klein et al., Nature 327: 70-73 (1987).

Agrobacterium tumefaciens-mediated transformation techniques are well described in the scientific literature. See, for example Horsch et al., Science 233: 496-498 (1984); Fraley et al., Proc. Natl. Acad. Sci. (USA) 80: 4803 (1983); and, Plant Molecular Biology: A Laboratory Manual, Chapter 8, Clark, Ed., Springer-Verlag, Berlin (1997). The DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616. Although Agrobacterium is useful primarily in dicots, certain monocots can be transformed by Agrobacterium. For instance, Agrobacterium transformation of maize is described in U.S. Pat. No. 5,550,318.

Other methods of transfection or transformation include (1) Agrobacterium rhizogenes-mediated transformation (see, e.g., Lichtenstein and Fuller In: Genetic Engineering, vol. 6, PWJ Rigby, Ed., London, Academic Press, 1987; and Lichtenstein, C. P., and Draper, J,. In: DNA Cloning, Vol. II, D. M. Glover, Ed., Oxford, IRI Press, 1985), Application PCT/US87/02512 (WO 88/02405 published Apr. 7, 1988) describes the use of A. rhizogenes strain A4 and its Ri plasmid along with A. tumefaciens vectors pARC8 or pARC16 (2) liposome-mediated DNA uptake (see, e.g., Freeman et al., Plant Cell Physiol. 25: 1353 (1984)), (3) the vortexing method (see, e.g., Kindle, Proc. Natl. Acad. Sci., (USA) 87: 1228 (1990).

DNA can also be introduced into plants by direct DNA transfer into pollen as described by Zhou et al., Methods in Enzymology, 101:433 (1983); D. Hess, Intern Rev. Cytol., 107:367 (1987); Luo et al., Plant Mol. Biol. Reporter, 6:165 (1988). Expression of polypeptide coding genes can be obtained by injection of the DNA into reproductive organs of a plant as described by Pena et al., Nature, 325.:274 (1987). DNA can also be injected directly into the cells of immature embryos and the rehydration of desiccated embryos as described by Neuhaus et al., Theor. Appl. Genet., 75:30 (1987); and Benbrook et al., in Proceedings Bio Expo 1986, Butterworth, Stoneham, Mass., pp. 27-54 (1986). A variety of plant viruses that can be employed as vectors are known in the art and include cauliflower mosaic virus (CaMV), geminivirus, brome mosaic virus, and tobacco mosaic virus.

B. Transfection of Prokaryotes, Lower Eukaryotes, and Animal Cells

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc. (1977).

### Transgenic Plant Regeneration

Plant cells which directly result or are derived from the nucleic acid introduction techniques can be cultured to regenerate a whole plant which possesses the introduced genotype. Such regeneration techniques often rely on manipulation of certain phytohormones in a tissue culture growth medium. Plants cells can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). This regeneration and growth process includes the steps of selection of transformant cells and shoots, rooting the transformant shoots and growth of the plantlets in soil. For maize cell culture and regeneration see generally, The Maize Handbook, Freeling and Walbot, Eds., Springer, N.Y. (1994); Corn and Corn Improvement, 3^{rd} edition, Sprague and Dudley Eds., American Society of Agronomy, Madison, Wisconsin (1988). For transformation and regeneration of maize see, Gordon-Kamm et al., The Plant Cell, 2:603-618 (1990).

The regeneration of plants containing the polynucleotide of the present invention and introduced by Agrobacterium from leaf explants can be achieved as described by Horsch etal., Science, 227:1229-1231 (1985). In this procedure, transformants are grown in the presence of a selection agent and in a medium that induces the regeneration of shoots in the plant species being transformed as described by Fraley et al., Proc. Natl. Acad. Sci. (U.S.A.), 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

Transgenic plants expressing a polynucleotide of the present invention can be screened for transmission of the nucleic acid of the present invention by, for example, standard immunoblot and DNA detection techniques. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Back-crossing to a parental plant and out-crossing with a non-transgenic plant are also contemplated.

### Modulating Polypeptide Levels and/or Composition

The present invention further provides a method for modulating (i.e., increasing or decreasing) the concentration or ratio of the polypeptides of the present invention in a plant or part thereof. Modulation can be effected by increasing or decreasing the concentration and/or the ratio of the polypeptides of the present invention in a plant. The method comprises introducing into a plant cell a recombinant expression cassette comprising a polynucleotide of the present invention as described above to obtain a transgenic plant cell, culturing the transgenic plant cell under transgenic plant cell growing conditions, and inducing or repressing expression of a polynucleotide of the present invention in the transgenic plant for a time sufficient to modulate concentration and/or the ratios of the polypeptides in the transgenic plant or plant part.

In some embodiments, the concentration and/or ratios of polypeptides of the present invention in a plant may be modulated by altering, in vivo or in vitro, the promoter of a gene to up- or down-regulate gene expression. In some embodiments, the coding regions of native genes of the present invention can be altered via substitution, addition, insertion, or deletion to decrease activity of the encoded enzyme. (See, e.g., Kmiec, U.S. Pat. No. 5,565,350; Zarling et al., PCT/US93/03868.) And in some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or ratios of polypeptides of the present invention in the plant. Plant forming conditions are well known in the art and discussed briefly, supra.

In general, concentration or the ratios of the polypeptides is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds which activate expression from these promoters are well known in the art. In preferred embodiments, the polypeptides of the present invention are modulated in monocots, particularly maize.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, Nucleic Acids Res. 15:8125 (1987)) and the 7-methylguanosine cap structure (Drummond et al., Nucleic Acids Res. 13:7375 (1985)). Negative elements include stable intramolecular 5′ UTR stem-loop structures (Muesing et al., Cell 48:691 (1987)) and AUG sequences or short open reading frames preceded by an appropriate AUG in the 5′ UTR (Kozak, supra, Rao et al., Mol. and Cell. Biol. 8:284 (1988)). Accordingly, the present invention provides 5′ and/or 3′ untranslated regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host such as to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res. 12: 387-395 (1984)) or MacVector 4.1 (Eastman Kodak Co., New Haven, Conn.). Thus, the present invention provides a codon usage frequency characteristic of the coding region of at least one of the polynucleotides of the present invention. The number of polynucleotides that can be used to determine a codon usage frequency can be any integer from 1 to the number of polynucleotides of the present invention as provided herein. Optionally, the polynucleotides will be full-length sequences. An exemplary number of sequences for statistical analysis can be at least 1, 5, 10, 20, 50, or 100.

### Sequence Shuffling

The present invention provides methods for sequence shuffling using polynucleotides of the present invention, and compositions resulting therefrom. Sequence shuffling is described in PCT publication No. WO 97/20078. See also, Zhang, J.-H., et al. Proc. Natl. Acad. Sci. USA 94:4504-4509 (1997). Generally, sequence shuffling provides a means for generating libraries of polynucleotides having a desired characteristic which can be selected or screened for. Libraries of recombinant polynucleotides are generated from a population of related sequence polynucleotides which comprise sequence regions which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be a decreased Kₘ and/or increased K_{cat} over the wild-type protein as provided herein. In other embodiments, a protein or polynucleotide generated from sequence shuffling will have a ligand binding affinity greater than the non-shuffled wild-type polynucleotide. The increase in such properties can be at least 110%, 120%, 130%, 140% or at least 150% of the wild-type value.

### Generic and Consensus Sequences

Polynucleotides and polypeptides of the present invention further include those having: (a) a generic sequence of at least two homologous polynucleotides or polypeptides, respectively, of the present invention; and, (b) a consensus sequence of at least three homologous polynucleotides or polypeptides, respectively, of the present invention. The generic sequence of the present invention comprises each species of polypeptide or polynucleotide embraced by the generic polypeptide or polynucleotide sequence, respectively. The individual species encompassed by a polynucleotide having an amino acid or nucleic acid consensus sequence can be used to generate antibodies or produce nucleic acid probes or primers to screen for homologs in other species, genera, families, orders, classes, phyla, or kingdoms. For example, a polynucleotide having a consensus sequence from a gene family of Zea mays can be used to generate antibody or nucleic acid probes or primers to other Gramineae species such as wheat, rice, or sorghum. Alternatively, a polynucleotide having a consensus sequence generated from orthologous genes can be used to identify or isolate orthologs of other taxa. Typically, a polynucleotide having a consensus sequence will be at least 9, 10, 15, 20, 25, 30, or 40 amino acids in length, or 20, 30, 40, 50, 100, or 150 nucleotides in length. As those of skill in the art are aware, a conservative amino acid substitution can be used for amino acids which differ amongst aligned sequence but are from the same conservative substitution group as discussed above. Optionally, no more than 1 or 2 conservative amino acids are substituted for each 10 amino acid length of consensus sequence.

Similar sequences used for generation of a consensus or generic sequence include any number and combination of allelic variants of the same gene, orthologous, or paralogous sequences as provided herein. Optionally, similar sequences used in generating a consensus or generic sequence are identified using the BLAST algorithm's smallest sum probability (P(N)). Various suppliers of sequence-analysis software are listed in chapter 7 of Current Protocols in Molecular Biology, F. M. Ausubel et al., Eds., Current Protocols, a joint venture between Greene Publishing Associates, Inc. and John Wiley & Sons, Inc. (Supplement 30). A polynucleotide sequence is considered similar to a reference sequence if the smallest sum probability in a comparison of the test nucleic acid to the reference nucleic acid is less than about 0.1, more preferably less than about 0.01, or 0.001, and most preferably less than about 0.0001, or 0.00001. Similar polynucleotides can be aligned and a consensus or generic sequence generated using multiple sequence alignment software available from a number of commercial suppliers such as the Genetics Computer Group's (Madison, Wis.) PILEUP software, Vector NTI's (North Bethesda, Md.) ALIGNX, or Genecode's (Ann Arbor, Mich.) SEQUENCHER. Conveniently, default parameters of such software can be used to generate consensus or generic sequences.

### Machine Applications

The present invention provides machines, data structures, and processes for modeling or analyzing the polynucleotides and polypeptides of the present invention.

A. Machines: Data, Data Structures, Processes, and Functions

The present invention provides a machine having a memory comprising: 1) data representing a sequence of a polynucleotide or polypeptide of the present invention, 2) a data structure which reflects the underlying organization and structure of the data and facilitates program access to data elements corresponding to logical sub-components of the sequence, 3) processes for effecting the use, analysis, or modeling of the sequence, and 4) optionally, a function or utility for the polynucleotide or polypeptide. Thus, the present invention provides a memory for storing data that can be accessed by a computer programmed to implement a process for effecting the use, analyses, or modeling of a sequence of a polynucleotide, with the memory comprising data representing the sequence of a polynucleotide of the present invention.

The machine of the present invention is typically a digital computer. The term “computer” includes one or several desktop or portable computers, computer workstations, servers (including intranet or internet servers), mainframes, and any integrated system comprising any of the above irrespective of whether the processing, memory, input, or output of the computer is remote or local, as well as any networking interconnecting the modules of the computer. The term “computer” is exclusive of computers of the United States Patent and Trademark Office or the European Patent Office when data representing the sequence of polypeptides or polynucleotides of the present invention is used for patentability searches.

The present invention contemplates providing as data a sequence of a polynucleotide of the present invention embodied in a computer readable medium. As those of skill in the art will be aware, the form of memory of a machine of the present invention, or the particular embodiment of the computer readable medium, are not critical elements of the invention and can take a variety of forms. The memory of such a machine includes, but is not limited to, ROM, or RAM, or computer readable media such as, but not limited to, magnetic media such as computer disks or hard drives, or media such as CD-ROMs, DVDs, and the like.

The present invention further contemplates providing a data structure that is also contained in memory. The data structure may be defined by the computer programs that define the processes (see below) or it may be defined by the programming of separate data storage and retrieval programs subroutines, or systems. Thus, the present invention provides a memory for storing a data structure that can be accessed by a computer programmed to implement a process for effecting the use, analysis, or modeling of a sequence of a polynucleotide. The memory comprises data representing a polynucleotide having the sequence of a polynucleotide of the present invention. The data is stored within memory. Further, a data structure, stored within memory, is associated with the data reflecting the underlying organization and structure of the data to facilitate program access to data elements corresponding to logical sub-components of the sequence. The data structure enables the polynucleotide to be identified and manipulated by such programs.

In a further embodiment, the present invention provides a data structure that contains data representing a sequence of a polynucleotide of the present invention stored within a computer readable medium. The data structure is organized to reflect the logical structuring of the sequence, so that the sequence is easily analyzed by software programs capable of accessing the data structure. In particular, the data structures of the present invention organize the reference sequences of the present invention in a manner which allows software tools to perform a wide variety of analyses using logical elements and sub-elements of each sequence.

An example of such a data structure resembles a layered hash table, where in one dimension the base content of the sequence is represented by a string of elements A, T, C, G and N. The direction from the 5′ end to the 3′ end is reflected by the order from the position 0 to the position of the length of the string minus one. Such a string, corresponding to a nucleotide sequence of interest, has a certain number of substrings, each of which is delimited by the string position of its 5′ end and the string position of its 3′ end within the parent string. In a second dimension, each substring is associated with or pointed to one or multiple attribute fields. Such attribute fields contain annotations to the region on the nucleotide sequence represented by the substring.

For example, a sequence under investigation is 520 bases long and represented by a string named SeqTarget. There is a minor groove in the 5′ upstream non-coding region from position 12 to 38, which is identified as a binding site for an enhancer protein HM-A, which in turn will increase the transcription of the gene represented by SeqTarget. Here, the substring is represented as (12, 38) and has the following attributes: [upstream uncoded], [minor groove], [HM-A binding] and [increase transcription upon binding by HM-A]. Similarly, other types of information can be stored and structured in this manner, such as information related to the whole sequence, e.g., whether the sequence is a full length viral gene, a mammalian house keeping gene or an EST from clone X, information related to the 3′ down stream non-coding region, e.g., hair pin structure, and information related to various domains of the coding region, e.g., Zinc finger.

This data structure is an open structure and is robust enough to accommodate newly generated data and acquired knowledge. Such a structure is also a flexible structure. It can be trimmed down to a 1-D string to facilitate data mining and analysis steps, such as clustering, repeat-masking, and HMM analysis. Meanwhile, such a data structure also can extend the associated attributes into multiple dimensions. Pointers can be established among the dimensioned attributes when needed to facilitate data management and processing in a comprehensive genomics knowledgebase. Furthermore, such a data structure is object-oriented. Polymorphism can be represented by a family or class of sequence objects, each of which has an internal structure as discussed above. The common traits are abstracted and assigned to the parent object, whereas each child object represents a specific variant of the family or class. Such a data structure allows data to be efficiently retrieved, updated and integrated by the software applications associated with the sequence database and/or knowledgebase.

The present invention contemplates providing processes for effecting analysis and modeling, which are described in the following section.

Optionally, the present invention further contemplates that the machine of the present invention will embody in some manner a utility or function for the polynucleotide or polypeptide of the present invention. The function or utility of the polynucleotide or polypeptide can be a function or utility for the sequence data, per se, or of the tangible material. Exemplary function or utilities include the name (per International Union of Biochemistry and Molecular Biology rules of nomenclature) or function of the enzyme or protein represented by the polynucleotide or polypeptide of the present invention; the metabolic pathway of the protein represented by the polynucleotide or polypeptide of the present invention; the substrate or product or structural role of the protein represented by the polynucleotide or polypeptide of the present invention; or, the phenotype (e.g., an agronomic or pharmacological trait) affected by modulating expression or activity of the protein represented by the polynucleotide or polypeptide of the present invention.

B. Computer Analysis and Modeling

The present invention provides a process of modeling and analyzing data representative of a polynucleotide or polypeptide sequence of the present invention. The process comprises entering sequence data of a polynucleotide or polypeptide of the present invention into a machine having a hardware or software sequence modeling and analysis system, developing data structures to facilitate access to the sequence data, manipulating the data to model or analyze the structure or activity of the polynucleotide or polypeptide, and displaying the results of the modeling or analysis. Thus, the present invention provides a process for effecting the use, analysis, or modeling of a polynucleotide sequence or its derived peptide sequence through use of a computer having a memory. The process comprises 1) placing into the memory data representing a polynucleotide having the sequence of a polynucleotide of the present invention, developing within the memory a data structure associated with the data and reflecting the underlying organization and structure of the data to facilitate program access to data elements corresponding to logical sub-components of the sequence, 2) programming the computer with a program containing instructions sufficient to implement the process for effecting the use, analysis, or modeling of the polynucleotide sequence or the peptide sequence, and, 3) executing the program on the computer while granting the program access to the data and to the data structure within the memory.

A variety of modeling and analytic tools are well known in the art and available commercially. Included amongst the modeling/analysis tools are methods to: 1) recognize overlapping sequences (e.g., from a sequencing project) with a polynucleotide of the present invention and create an alignment called a “contig”; 2) identify restriction enzyme sites of a polynucleotide of the present invention; 3) identify the products of a T1 ribonuclease digestion of a polynucleotide of the present invention; 4) identify PCR primers with minimal self-complementarity; 5) compute pairwise distances between sequences in an alignment, reconstruct phylogentic trees using distance methods, and calculate the degree of divergence of two protein coding regions; 6) identify patterns such as coding regions, terminators, repeats, and other consensus patterns in polynucleotides of the present invention; 7) identify RNA secondary structure; 8) identify sequence motifs, isoelectric point, secondary structure, hydrophobicity, and antigenicity in polypeptides of the present invention; 9) translate polynucleotides of the present invention and backtranslate polypeptides of the present invention; and 10) compare two protein or nucleic acid sequences and identifying points of similarity or dissimilarity between them.

The processes for effecting analysis and modeling can be produced independently or obtained from commercial suppliers. Exemplary analysis and modeling tools are provided in products such as InforMax's (Bethesda, Md.) Vector NTI Suite (Version 5.5), Intelligenetics' (Mountain View, Calif.) PC/Gene program, and Genetics Computer Group's (Madison, Wis.) Wisconsin Package (Version 10.0); these tools, and the functions they perform, (as provided and disclosed by the programs and accompanying literature) are incorporated herein by reference and are described in more detail in section C which follows.

Thus, in a further embodiment, the present invention provides a machine-readable media containing a computer program and data, comprising a program stored on the media containing instructions sufficient to implement a process for effecting the use, analysis, or modeling of a representation of a polynucleotide or peptide sequence. The data stored on the media represents a sequence of a polynucleotide having the sequence of a polynucleotide of the present invention. The media also includes a data structure reflecting the underlying organization and structure of the data to facilitate program access to data elements corresponding to logical sub-components of the sequence, the data structure being inherent in the program and in the way in which the program organizes and accesses the data.

C. Homology Searches

As an example of such a comparative analysis, the present invention provides a process of identifying a candidate homologue (i.e., an ortholog or paralog) of a polynucleotide or polypeptide of the present invention. The process comprises entering sequence data of a polynucleotide or polypeptide of the present invention into a machine having a hardware or software sequence analysis system, developing data structures to facilitate access to the sequence data, manipulating the data to analyze the structure the polynucleotide or polypeptide, and displaying the results of the analysis. A candidate homologue has statistically significant probability of having the same biological function (e.g., catalyzes the same reaction, binds to homologous proteins/nucleic acids, has a similar structural role) as the reference sequence to which it is compared. Accordingly, the polynucleotides and polypeptides of the present invention have utility in identifying homologs in animals or other plant species, particularly those in the family Gramineae such as, but not limited to, sorghum, wheat, or rice.

The process of the present invention comprises obtaining data representing a polynucleotide or polypeptide test sequence. Test sequences can be obtained from a nucleic acid of an animal or plant. Test sequences can be obtained directly or indirectly from sequence databases including, but not limited to, those such as: GenBank, EMBL, GenSeq, SWISS-PROT, or those available on-line via the UK Human Genome Mapping Project (HGMP) GenomeWeb. In some embodiments the test sequence is obtained from a plant species other than maize whose function is uncertain but will be compared to the test sequence to determine sequence similarity or sequence identity. The test sequence data is entered into a machine, such as a computer, containing: i) data representing a reference sequence and, ii) a hardware or software sequence comparison system to compare the reference and test sequence for sequence similarity or identity.

Exemplary sequence comparison systems are provided for in sequence analysis software such as those provided by the Genetics Computer Group (Madison, Wis.) or InforMax (Bethesda, Md.), or Intelligenetics (Mountain View, Calif.). Optionally, sequence comparison is established using the BLAST or GAP suite of programs. Generally, a smallest sum probability value (P(N)) of less than 0.1, or alternatively, less than 0.01, 0.001, 0.0001, or 0.00001 using the BLAST 2.0 suite of algorithms under default parameters identifies the test sequence as a candidate homologue (i.e., an allele, ortholog, or paralog) of the reference sequence. Those of skill in the art will recognize that a candidate homologue has an increased statistical probability of having the same or similar function as the gene/protein represented by the test sequence.

The reference sequence can be the sequence of a polypeptide or a polynucleotide of the present invention. The reference or test sequence is each optionally at least 25 amino acids or at least 100 nucleotides in length. The length of the reference or test sequences can be the length of the polynucleotide or polypeptide described, respectively, above in the sections entitled “Nucleic Acids” (particularly section (g)), and “Proteins”. As those of skill in the art are aware, the greater the sequence identity/similarity between a reference sequence of known function and a test sequence, the greater the probability that the test sequence will have the same or similar function as the reference sequence. The results of the comparison between the test and reference sequences are outputted (e.g., displayed, printed, recorded) via any one of a number of output devices and/or media (e.g., computer monitor, hard copy, or computer readable medium).

### Detection of Nucleic Acids

The present invention further provides methods for detecting a polynucleotide of the present invention in a nucleic acid sample suspected of containing a polynucleotide of the present invention, such as a plant cell lysate, particularly a lysate of maize. In some embodiments, a cognate gene of a polynucleotide of the present invention or portion thereof can be amplified prior to the step of contacting the nucleic acid sample with a polynucleotide of the present invention. The nucleic acid sample is contacted with the polynucleotide to form a hybridization complex. The polynucleotide hybridizes under stringent conditions to a gene encoding a polypeptide of the present invention. Formation of the hybridization complex is used to detect a gene encoding a polypeptide of the present invention in the nucleic acid sample. Those of skill will appreciate that an isolated nucleic acid comprising a polynucleotide of the present invention should lack cross-hybridizing sequences in common with non-target genes that would yield a false positive result. Detection of the hybridization complex can be achieved using any number of well known methods. For example, the nucleic acid sample, or a portion thereof, may be assayed by hybridization formats including but not limited to, solution phase, solid phase, mixed phase, or in situ hybridization assays.

Detectable labels suitable for use in the present invention include any composition detectable by spectroscopic, radioisotopic, photochemical, biochemical, immunochemical, electrical, optical or chemical means. Useful labels in the present invention include biotin for staining with labeled streptavidin conjugate, magnetic beads, fluorescent dyes, radiolabels, enzymes, and calorimetric labels. Other labels include ligands which bind to antibodies labeled with fluorophores, chemiluminescent agents, and enzymes. Labeling the nucleic acids of the present invention is readily achieved such as by the use of labeled PCR primers.

Although the present invention has been described in some detail by way of illustration and example for purposes of clarity of understanding, it will be obvious that certain changes and modifications may be practiced within the scope of the appended claims.

### EXAMPLE 1

This example describes the construction of a cDNA library.

Total RNA can be isolated from maize tissues with TRIzol Reagent (Life Technology Inc. Gaithersburg, Md.) using a modification of the guanidine isothiocyanate/acid-phenol procedure described by Chomczynski and Sacchi (Chomczynski, P., and Sacchi, N. Anal. Biochem. 162, 156 (1987)). In brief, plant tissue samples is pulverized in liquid nitrogen before the addition of the TRIzol Reagent, and then further homogenized with a mortar and pestle. Addition of chloroform followed by centrifugation is conducted for separation of an aqueous phase and an organic phase. The total RNA is recovered by precipitation with isopropyl alcohol from the aqueous phase.

The selection of poly(A)+ RNA from total RNA can be performed using PolyATact system (Promega Corporation. Madison, Wis.). Biotinylated oligo(dT) primers are used to hybridize to the 3′ poly(A) tails on mRNA. The hybrids are captured using streptavidin coupled to paramagnetic particles and a magnetic separation stand. The mRNA is then washed at high stringency conditions and eluted by RNase-free deionized water.

cDNA synthesis and construction of unidirectional cDNA libraries can be accomplished using the SuperScript Plasmid System (Life Technology Inc. Gaithersburg, Md.). The first strand of cDNA is synthesized by priming an oligo(dT) primer containing a Not I site. The reaction is catalyzed by SuperScript Reverse Transcriptase II at 45° C. The second strand of cDNA is labeled with alpha-³²P-dCTP and a portion of the reaction analyzed by agarose gel electrophoresis to determine cDNA sizes. cDNA molecules smaller than 500 base pairs and unligated adapters are removed by Sephacryl-S400 chromatography. The selected cDNA molecules are ligated into pSPORT1 vector in between of Not I and Sal I sites.

Alternatively, cDNA libraries can be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

### EXAMPLE 2

This method describes construction of a full-length enriched cDNA library.

An enriched full-length cDNA library can be constructed using one of two variations of the method of Carninci et al. Genomics 37: 327-336, 1996. These variations are based on chemical introduction of a biotin group into the diol residue of the 5′ cap structure of eukaryotic mRNA to select full-length first strand cDNA. The selection occurs by trapping the biotin residue at the cap sites using streptavidin-coated magnetic beads followed by RNase I treatment to eliminate incompletely synthesized cDNAs. Second strand cDNA is synthesized using established procedures such as those provided in Life Technologies' (Rockville, Md.) “SuperScript Plasmid System for cDNA Synthesis and Plasmid Cloning” kit. Libraries made by this method have been shown to contain 50% to 70% full-length cDNAs.

The first strand synthesis methods are detailed below. An asterisk denotes that the reagent was obtained from Life Technologies, Inc.

A. First Strand cDNA Synthesis Method 1 (with Trehalose)

| | |
|---|---|
| mRNA (10 ug) | 25 μl |
| *Not I primer (5 ug) | 10 μl |
| *5x 1^{st} strand buffer | 43 μl |
| *0.1 m DTT | 20 μl |
| *dNTP mix 10 mm | 10 μl |
| BSA 10 ug/μl | 1 μl |
| Trehalose (saturated) | 59.2 μl |
| RNase inhibitor (Promega) | 1.8 μl |
| *Superscript II RT 200 u/μl | 20 μl |
| 100% glycerol | 18 μl |
| Water | 7 μl |

The mRNA and Not I primer are mixed and denatured at 65° C. for 10 min. They are then chilled on ice and other components added to the tube. Incubation is at 45° C. for 2 min. Twenty microliters of RT (reverse transcriptase) is added to the reaction and start program on the thermocycler (MJ Research, Waltham, Mass.):

| | |
|---|---|
| Step 1 | 45° C. 10 min |
| Step 2 | 45° C. −0.3° C./cycle, 2 seconds/cycle |
| Step 3 | go to 2 for 33 cycles |
| Step 4 | 35° C. 5 min |
| Step 5 | 45° C. 5 min |
| Step 6 | 45° C. 0.2° C./cycle, 1 sec/cycle |
| Step 7 | go to 7 for 49 cycles |
| Step 8 | 55° C. 0.1° C./cycle, 12 sec/cycle |
| Step 9 | go to 8 for 49 cycles |
| Step 10 | 55° C. 2 min |
| Step 11 | 60° C. 2 min |
| Step 12 | go to 11 for 9 times |
| Step 13 | 4° C. forever |
| Step 14 | end |

B. First Strand cDNA Synthesis Method 2

| mRNA (10 μg) | 25 μl |
|---|---|
| water | 30 μl |
| *Not I adapter primer (5 μg) | 10 μl |
| 65° C. for 10 min, chill on ice, then add following reagents, | |
| *5x first buffer | 20 μl |
| *0.1 M DTT | 10 μl |
| *10 mM dNTP mix | 5 μl |

Incubate at 45° C. for 2 min, then add 10 μl of *Superscript II RT (200 u/μl), start the following program:

| | |
|---|---|
| Step 1 | 45° C. for 6 sec, −0.1° C./cycle |
| Step 2 | go to 1 for 99 additional cycles |
| Step 3 | 35° C. for 5 min |
| Step 4 | 45° C. for 60 min |
| Step 5 | 50° C. for 10 min |
| Step 6 | 4° C. forever |
| Step 7 | end |

After the 1^{st} strand cDNA synthesis, the DNA is extracted by phenol according to standard procedures, and then precipitated in NaOAc and ethanol, and stored in −20° C.

C. Oxidization of the Diol Group of mRNA for Biotin Labeling

First strand cDNA is spun down and washed once with 70% EtOH. The pellet resuspended in 23.2 μl of DEPC treated water and put on ice. Prepare 100 mM of NalO4 freshly, and then add the following reagents:

| | |
|---|---|
| mRNA:1^{st} cDNA (start with 20 μg mRNA) | 46.4 μl |
| 100 mM NalO4 (freshly made) | 2.5 μl |
| NaOAc 3 M pH 4.5 | 1.1 μl |

To make 100 mM NalO4, use 21.39 μg of NalO4 for 1 μl of water. Wrap the tube in a foil and incubate on ice for 45 min. After the incubation, the reaction is then precipitated in:

| | |
|---|---|
| 5 M NaCl | 10 μl |
| 20% SDS | 0.5 μl |
| isopropanol | 61 μl |

Incubate on ice for at least 30 min, then spin it down at max speed at 4° C. for 30 min and wash once with 70% ethanol and then 80% EtOH.

D. Biotinylation of the mRNA Diol Group

Resuspend the DNA in 110 μl DEPC treated water, then add the following reagents:

| | |
|---|---|
| 20% SDS | 5 μl |
| 2 M NaOAc pH 6.1 | 5 μl |
| 10 mm biotin hydrazide (freshly made) | 300 μl |

Wrap in a foil and incubate at room temperature overnight.

E. RNase I Treatment

Precipitate DNA in:

| | |
|---|---|
| 5 M NaCl | 10 μl |
| 2 M NaOAc pH 6.1 | 75 μl |
| biotinylated mRNA:cDNA | 420 μl |
| 100% EtOH (2.5 Vol) | 1262.5 μl |

(Perform this precipitation in two tubes and split the 420 μl of DNA into 210 μl each, add 5 μl of 5M NaCl, 37.5 μl of 2M NaOAc pH 6.1, and 631.25 μl of 100% EtOH). Store at −20° C. for at least 30 min. Spin the DNA down at 4° C. at maximal speed for 30 min. and wash with 80% EtOH twice, then dissolve DNA in 70 μl RNase free water. Pool two tubes and end up with 140 μl. Add the following reagents:

| | |
|---|---|
| RNase One 10 U/μl | 40 μl |
| 1^{st} cDNA:RNA | 140 μl |
| 10X buffer | 20 μl |

Incubate at 37° C. for 15 Min.

Add 5 μl of 40 μg/μl yeast tRNA to each sample for capturing.

F. Full length 1^{st} cDNA Capturing

Blocking the beads with yeast tRNA:

| | |
|---|---|
| Beads | 1 ml |
| Yeast tRNA 40 μg/μl | 5 μl |

Incubate on ice for 30 min with mixing, wash 3 times with 1 ml of 2M NaCl, 50 mmEDTA, pH 8.0.

Resuspend the beads in 800 μl of 2M NaCl, 50 mm EDTA, pH 8.0, add RNase I treated sample 200 μl, and incubate the reaction for 30 min at room temperature.

Capture the beads using the magnetic stand, save the supernatant, and start following washes:

2 washes with 2M NaCl, 50 mm EDTA, pH 8.0, 1 ml each time,

1 wash with 0.4% SDS, 50 μg/ml tRNA,

1 wash with 10 mm Tris-Cl pH 7.5, 0.2 mm EDTA, 10 mm NaCl, 20% glycerol,

1 wash with 50 μg/ml tRNA,

1 wash with 1^{st} cDNA buffer

G. Second Strand cDNA Synthesis

Resuspend the beads in:

| | |
|---|---|
| *5X first buffer | 8 μl |
| *0.1 mM DTT | 4 μl |
| *10 mm dNTP mix | 8 μl |
| *5X 2nd buffer | 60 μl |
| *E. coli Ligase 10 U/μl | 2 μl |
| *E. coli DNA polymerase 10 U/μl | 8 μl |
| *E. coli RNaseH 2 U/μl | 2 μl |
| P32 dCTP 10 μci/μl | 2 μl |
| Or water up to 300 μl | 208 μl |

Incubate at 16° C. for 2 hr with mixing the reaction in every 30 min.

Add 4 μl of T4 DNA polymerase and incubate for additional 5 min at 16° C.

Elute 2^{nd} cDNA from the beads.

Use a magnetic stand to separate the 2^{nd} cDNA from the beads, then resuspend the beads in 200 μl of water, and then separate again, pool the samples (about 500 μl),

Add 200 μl of water to the beads, then 200 μl of phenol:chloroform, vortex, and spin to separate the sample with phenol.

Pool the DNA together (about 700 μl) and use phenol to clean the DNA again, DNA is then precipitated in 2 μg of glycogen and 0.5 vol of 7.5M NH4OAc and 2 vol of 100% EtOH. Precipitate overnight. Spin down the pellet and wash with 70% EtOH, air-dry the pellet.

| | | | |
|---|---|---|---|
| DNA | 250 μl | DNA | 200 μl |
| 7.5 M NH4OAc | 125 μl | 7.5 M NH4OAc | 100 μl |
| 100% EtOH | 750 μl | 100% EtOH | 600 μl |
| glycogen 1 μg/μl | 2 μl | glycogen 1 μg/μl | 2 μl |

H. Sal Adapter Ligation

Resuspend the pellet in 26 μl of water and use 1 μl for TAE gel.

Set up reaction as following:

| | |
|---|---|
| 2^{nd} strand cDNA | 25 μl |
| *5X T4 DNA ligase buffer | 10 μl |
| *Sal I adapters | 10 μl |
| *T4 DNA ligase | 5 μl |

Mix gently, incubate the reaction at 16° C. overnight.

Add 2 μl of ligase second day and incubate at room temperature for 2 hrs (optional).

Add 50 μl water to the reaction and use 100 μl of phenol to clean the DNA, 90 μl of the upper phase is transferred into a new tube and precipitate in:

| | |
|---|---|
| Glycogen 1 μg/μl | 2 μl |
| Upper phase DNA | 90 μl |
| 7.5 M NH4OAc | 50 μl |
| 100% EtOH | 300 μl |

precipitate at −20° C. overnight

Spin down the pellet at 4° C. and wash in 70% EtOH, dry the pellet.

I. Not I digestion

| | |
|---|---|
| 2^{nd} cDNA | 41 μl |
| *Reaction 3 buffer | 5 μl |
| *Not I 15 u/μl | 4 μl |

Mix gently and incubate the reaction at 37° C. for 2 hr.

Add 50 μl of water and 100 μl of phenol, vortex, and take 90 μl of the upper phase to a new tube, then add 50 μl of NH₄0Ac and 300 μl of EtOH. Precipitate overnight at −20° C.

Cloning, ligation, and transformation are performed per the Superscript cDNA synthesis kit.

### EXAMPLE 3

This example describes cDNA sequencing and library subtraction.

Individual colonies can be picked and DNA prepared either by PCR with M13 forward primers and M13 reverse primers, or by plasmid isolation. cDNA clones can be sequenced using M13 reverse primers.

cDNA libraries are plated out on 22×22 cm² agar plate at density of about 3,000 colonies per plate. The plates are incubated in a 37° C. incubator for 12-24 hours. Colonies are picked into 384-well plates by a robot colony picker, Q-bot (GENETIX Limited). These plates are incubated overnight at 37° C. Once sufficient colonies are picked, they are pinned onto 22×22 cm² nylon membranes using Q-bot. Each membrane holds 9,216 or 36,864 colonies. These membranes are placed onto an agar plate with an appropriate antibiotic. The plates are incubated at 37° C. overnight.

After colonies are recovered on the second day, these filters are placed on filter paper prewetted with denaturing solution for four minutes, then incubated on top of a boiling water bath for an additional four minutes. The filters are then placed on filter paper prewetted with neutralizing solution for four minutes. After excess solution is removed by placing the filters on dry filter papers for one minute, the colony side of the filters is placed into Proteinase K solution, incubated at 37° C. for 40-50 minutes. The filters are placed on dry filter papers to dry overnight. DNA is then cross-linked to nylon membrane by UV light treatment

Colony hybridization is conducted as described by Sambrook, J., Fritsch, E. F. and Maniatis, T., (in Molecular Cloning: A laboratory Manual, 2^{nd} Edition). The following probes can be used in colony hybridization:

1. First strand cDNA from the same tissue as the library was made from to remove the most redundant clones.

2. 48-192 most redundant cDNA clones from the same library based on previous sequencing data.

3. 192 most redundant cDNA clones in the entire maize sequence database.

4. A Sal-A20 oligo nucleotide: TCG ACC CAC GCG TCC GAA AAA AAA AAA AAA MA AAA, SEQ ID NO.31, removes clones containing a poly A tail but no cDNA.

5. cDNA clones derived from rRNA.

The image of the autoradiography is scanned into computer and the signal intensity and cold colony addresses of each colony is analyzed. Re-arraying of cold-colonies from 384 well plates to 96 well plates is conducted using Q-bot.

### EXAMPLE 4

This example describes identification of the gene from a computer homology search.

Gene identities can be determined by conducting BLAST (Basic Local Alignment Search Tool; Altschul, S. F., et al., (1993) J. Mol. Biol. 215:403-410) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences are analyzed for similarity to all publicly available DNA sequences contained in the “nr” database using the BLASTN algorithm. The DNA sequences are translated in all reading frames and compared for similarity to all publicly available protein sequences contained in the “nr” database using the BLASTX algorithm (Gish, W. and States, D. J. Nature Genetics 3:266-272 (1993)) provided by the NCBI. In some cases, the sequencing data from two or more clones containing overlapping segments of DNA are used to construct contiguous DNA sequences.

Sequence alignments and percent identity calculations can be performed using the Megalign program of the LASERGENE bioinformatics computing suite (DNASTAR Inc., Madison, Wis.). Multiple alignments of the sequences can be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

Other methods of sequence alignment and percent identity analysis known to those of skill in the art, including those disclosed herein, can also be employed.

### EXAMPLE 5

This example describes expression of transgenes in monocot cells.

A transgene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue; Stratagene). Bacterial transformants can be screened by restriction enzyme digestion of plasmid DNA and limited nucleotide sequence analysis using the dideoxy chain termination method (Sequenase DNA Sequencing Kit; U. S. Biochemical). The resulting plasmid construct would comprise a transgene encoding, in the 5′ to 3′ direction, the maize 27 kD zein promoter, a cDNA fragment encoding the instant polypeptides, and the 10 kD zein 3′ region.

The transgene described above can then be introduced into maize cells by the following procedure. Immature maize embryos can be dissected from developing caryopses derived from crosses of the inbred maize lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (Hoechst Ag, Frankfurt, Germany) or equivalent may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten pg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the maize tissue with a Biolistic PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covers a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### EXAMPLE 6

This example describes expression of transgenes in dicot cells.

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), SmaI, KpnI and XbaI. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic PDS1000/HE instrument (helium retrofit) can be used for these transformations.

A selectable marker gene which can be used to facilitate soybean transformation is a transgene composed of the 35S promoter from Cauliflower Mosaic Virus (Odell etal.(1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptides and the phaseolin 3′ region can be isolated as a restriction fragment. This fragment can then be inserted into a unique restriction site of the vector carrying the marker gene.

To 50 μL of a 60 mg/mL 1 μmgold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### EXAMPLE 7

This example describes expression of a transgene in microbial cells. The cDNAs encoding the instant polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% NuSieve GTG low melting agarose gel (FMC). Buffer and agarose contain 10 μg/ml ethidium bromide for visualization of the DNA fragment. The fragment can then be purified from the agarose gel by digestion with GELase (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25° C. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One microgram of protein from the soluble fraction of the culture can be separated by SDS-polyacrylamide gel electrophoresis. Gels can be observed for protein bands migrating at the expected molecular weight.

### EXAMPLE 8

### Isolation of the CesA10, 11, and 12 Genes and their Relevanc to Cell Wall Synthesis and Stalk Strength in Maize

All three genes were isolated from a library made from the zone of an elongating corn stalk internode between the elongation zone and the most mature part of the internode, the “transition zone”. The library was made, subtracted, and sequenced as described in the preceding Examples 1, 2, and 3. A genomic database search was conducted as described in Example 4. Derived polypeptide sequences of all the Expressed Tag Sequences (ESTs) showing homology to the 1 kb 5′-end of any of the 9 previously known ZmCesA genes were aligned with the protein sequences of the latter. The sequences that did not fully match any of the known genes were sequenced from both ends of the respective cDNA clones. Three new, full-length genes, ZmCesA10 (SEQ ID NO. 25), ZmCesA11 (SEQ ID NO. 27), and ZmCesA12 (SEQ ID NO. 29) were isolated by this method.

The polypeptide sequences of the three genes derived from the cDNA sequences (SEQ ID NOs. 26, 28, and 30, respectively) clustered with the CesA genes from other species where they are known to be involved in secondary wall formation (FIG. 4). AtCesA7 and AtCesA8 have been found to make secondary wall in the vascular bundles (Taylor Neil, G., Laurie, S., and Turner Simon, R. (2000). Multiple cellulose synthase catalytic subunits are required for cellulose synthesis in Arabidopsis. Plant Cell, 2000; 12, 2529-2539.). Retrotransposon insertions into OsCesA4 and OsCesA7 resulted in a brittle culm phenotype in rice (Katsuyuki Tanaka, Akio Miyao, Kazumasa Murata, Katsura Onosato, Naoko Kojima, Yumiko Yamashita, Mayuko Harada, Takuji Sasaki, Hirohiko Hirochika, 2002, Analysis of rice brittle mutants caused by disruption of cellulose synthase genes OsCesA4 and OsCesA11 with the retrotransposon tos 17. Plant, Animal & Microbe Genomes X. San Diego, Calif. Abs.# 324). Each of the genes, ZmCesA 10, 11, or 12, groups with one or the other CesA gene from Arabidopsis or rice known to be involved in secondary wall formation and thus in determining tissue strength (FIG. 4). The CesA genes derived from the tissues specializing in secondary wall formation from other species (Gossypium, Zinnia, Populus) also group into the same clades with the aforementioned genes.

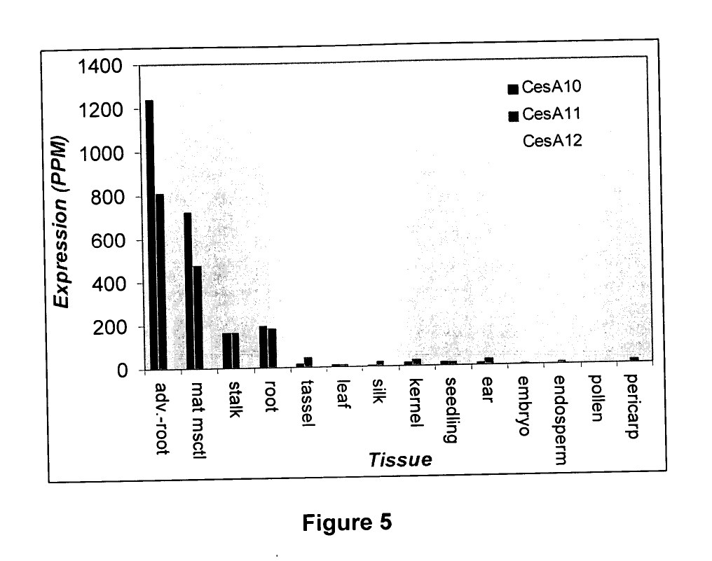

Further evidence that the maize genes are involved in secondary wall formation and thus in determining stalk strength was obtained from their expression pattern using the Massively Parallel Signature Sequencing (MPSS) technology (Brenner, S., Williams Steven, R., Vermaas Eric, H., Storck, T., Moon, K., McCollum, C., Mao Jen, I., Luo, S., Kirchner James, J., Eletr, S., DuBridge Robert, B., Burcham, T., and Albrecht, G. (2000), In vitro cloning of complex mixtures of DNA on microbeads: Physical separation of differentially expressed cDNAs. Proceedings-of-the-National-Academy-of-Sciences-of-the-United-States-of-America, Feb. 15, 2000; 97, 1665-1670.; see also Brenner, S., Johnson, M., Bridgham, J., Golda, G., Lloyd David, H., Johnson, D., Luo, S., McCurdy, S., Foy, M., Ewan, M., Roth, R., George, D., Eletr, S., Albrecht, G., Vermaas, E., Williams Steven, R., Moon, K., Burcham, T., Pallas, M., DuBridge Robert, B., Kirchner, J., Fearon, K., Mao Jen, i., and Corcoran, K. (2000), Gene expression analysis by massively parallel signature sequencing (MPSS) on microbead arrays. Nature-Biotechnolog, [print] June, 2000; 18, 630-634.; see also Dhugga, K. S. (2001), Building the wall: genes and enzyme complexes for polysaccharide synthases, Curr. Opin. Plant Biol. 4, 488-493.). All three genes are expressed in the tissues rich in cell wall content, supporting their involvement in secondary wall formation as deduced from their relationship to the genes from the other, aforementioned species know to play this role (FIG. 5). All three genes are expressed nearly identically across multiple tissues as seen from the correlation coefficient matrix (Table 3), further strengthening the argument that they are involved in secondary wall formation in the vascular bundles and thus in determining tissue strength.

Correlation among the expression level of the different CesA genes from maize as studied from Lynx are shown in Table 3.

TABLE 3

| | CesA1 | C sA2 | CesA3 | CesA4 | CesA5 | CesA6 | CesA7 | CesA8 | CesA10 | CesA11 | CesA12 |
|---|---|---|---|---|---|---|---|---|---|---|---|
| CesA1 | 1 | | | | | | | | | | |
| CesA2 | 0.59 | 1.00 | | | | | | | | | |
| CesA3 | 0.07 | −0.15 | 1.00 | | | | | | | | |
| CesA4 | 0.44 | 0.55 | −0.10 | 1.00 | | | | | | | |
| CesA5 | −0.20 | −0.29 | 0.45 | −0.33 | 1.00 | | | | | | |
| CesA6 | 0.56 | 0.14 | 0.14 | 0.08 | −0.13 | 1.00 | | | | | |
| CesA7 | 0.68 | 0.76 | −0.06 | 0.57 | −0.29 | 0.32 | 1.00 | | | | |
| CesA8 | 0.59 | 0.73 | −0.16 | 0.58 | −0.36 | 0.26 | 0.61 | 1.00 | | | |
| CesA10 | 0.27 | 0.37 | −0.27 | 0.33 | −0.26 | 0.02 | 0.33 | 0.36 | 1.00 | | |
| CesA11 | 0.39 | 0.47 | −0.22 | 0.38 | −0.28 | 0.11 | 0.40 | 0.42 | 0.95 | 1.00 | |
| CesA12 | 0.34 | 0.49 | −0.27 | 0.37 | −0.31 | 0.08 | 0.44 | 0.45 | 0.95 | 0.95 | 1.00 |

The correlation matrix was derived from the expression, measured in PPM, from 65 different tissue libraries. Note the nearly perfect correlation among the expression pattern of the CesA10, 11, and 12 genes.

### EXAMPLE 9

This example describes a procedure to identify plants containing Mu inserted into genes of interest and a strategy to identify the function of those genes. This procedure was also described in U.S. patent application Ser. No. 09/371,383 which disclosed members of the same gene family as the present application. One of skill in the art could readily conceive of use of this procedure with the any of the Cellulose Synthase (CesA) sequences disclosed in the current application. The current example is based on work with the CesA11 gene, identified as SEQ ID No. 27 herein.

The Trait Utility System for Corn (TUSC) is a method that employs genetic and molecular techniques to facilitate the study of gene function in maize. Studying gene function implies that the gene's sequence is already known, thus the method works in reverse: from sequence to phenotype. This kind of application is referred to as “reverse genetics”, which contrasts with “forward” methods that are designed to identify and isolate the gene(s) responsible for a particular trait (phenotype).

Pioneer Hi-Bred International, Inc., has a proprietary collection of maize genomic DNA from approximately 42,000 individual F₁ plants (Reverse genetics for maize, Meeley, R. and Briggs, S., 1995, Maize Genet. Coop. Newslett. 69:67, 82). The genome of each of these individuals contains multiple copies of the transposable element family, Mutator (Mu). The Mu family is highly mutagenic; in the presence of the active element Mu-DR, these elements transpose throughout the genome, inserting into genic regions, and often disrupting gene function. By collecting genomic DNA from a large number (42,000) of individuals, Pioneer has assembled a library of the mutagenized maize genome.

Mu insertion events are predominantly heterozygous; given the recessive nature of most insertional mutations, the F₁ plants appear wild-type. Each of the F₁ plants is selfed to produce F₂ seed, which is collected. In generating the F₂ progeny, insertional mutations segregate in a Mendelian fashion so are useful for investigating a mutant allele's effect on the phenotype. The TUSC system has been successfully used by a number of laboratories to identify the function of a variety of genes (Cloning and characterization of the maize An1 gene, Bensen, R. J., et al., 1995, Plant Cell 7:75-84; Diversification of C-function activity in maize flower development, Mena, M., et al., 1996, Science 274:1537-1540; Analysis of a chemical plant defense mechanism in grasses, Frey, M., et al., 1997, Science 277:696-699; The control of maize spikelet meristem fate by the APETALA2-like gene Indeterminate spikelet 1, Chuck, G., Meeley, R. B., and Hake, S., 1998, Genes & Development 12:1145-1154; A SecY homologue is required for the elaboration of the chloroplast thylakoid membrane and for normal chloroplast gene expression, Roy, L. M. and Barkan, A., 1998, J. Cell Biol. 141:1-11).

PCR Screening for Mu insertions in CesA11:

Two primers were designed from within the CesA11 cDNA and designated as gene-specific primers (GSPs):

Forward primer:

(GSP1/SEQ ID NO. 32)

5′-TACGATGAGTACGAGAGGTCCATGCTCA-3′
Reverse primer:

(GSP2/SEQ ID NO. 33)

5′-GGCAAAAGCCCAGATGCGAGATAGAC-3′
Mu TIR primer:

(SEQ ID NO. 34)

5′-AGAGMGCCAACGCCAWCGCCTCYATTTCGTC-3′

Pickoligo was used to select primers for PCR. This program chooses the Tm according to the following equation:

Tm=[((GC*3+AT*2)*37−562)/length]−5

PCR reactions were run with an annealing temperature of 62° C. and a thermocycling profile as follows:

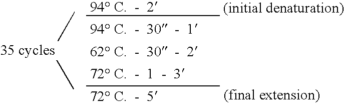

Gel electrophoresis of the PCR products confirmed that there was no false priming in single primer reactions and that only one fragment was amplified in paired GSP reactions.

The genomic DNA from 42,000 plants, combined into pools of 48 plants each, was subjected to PCR with either GSP1 or GSP2 and Mu TIR. The pools that were confirmed to be positive by dot-blot hybridization using CesA11 cDNA as a probe were subjected to gel-blot analysis in order to determine the size of fragments amplified. The pools in which clean fragments were identified were subjected to further analysis to identify the individual plants within those pools that contained Mu insertion(s).

Seed from F₁ plants identified in this manner was planted in the field. Leaf discs from twenty plants in each F₂ row were collected and genomic DNA was isolated. The same twenty plants were selfed and the F₃ seed saved. Pooled DNA (from 20 plants) from each of twelve rows was subjected to PCR using GSP1 or GSP2 and Mu TIR primer as mentioned above. Three pools identified to contain Mu insertions were subjected to individual plant analysis and homozygotes identified. The Mu insertion sites with the surrounding signature sequences are identified below:

| | |
|---|---|
| Allele 1: | 5′-TGGCGGCCG(SEQ ID NO. 35)-Mu-TCTGAAATG(SEQ ID NO. 36)-3′ |
| Allele 2: | 5′-GCCCACAAG(SEQ ID NO. 37)-Mu-CATCCTGGT(SEQ ID NO. 38)-3′ |
| Allele 3: | 5′-GTGTTCTTC(SEQ ID NO. 39)-Mu-GCCATGTGG(SEQ ID NO. 40)-3′ |

All three insertions are within 500 nucleotides of each other in the open reading frame, suggesting that this region in the gene might represent a hot spot for Mu insertion. One of the insertions, allele 1, is in the region upstream of the predicted six transmembrane domains near the C-terminal end of the protein. Each of these insertions is expected to inactivate the gene since they are all in the exonic regions of the gene.

### EXAMPLE 10

This example describes the method used to measure mechanical strength of the maize stalks as well as the effect of the overexpression of different CesA genes on stalk strength. The mechanical strength of the mature corn stalks was measured with an electromechanical test system. The internodes below the ear were subjected to a 3-point bend test using an Instron, model 4411 (Instron Corporation, 100 Royall Street, Canton, Mass. 02021), with a span-width of 200 mm between the anchoring points and a speed of 200 mm/min of the 3^{rd} point attached to a load cell. For measuring rind puncture strength, a needle was mounted on the load cell of the Instron and the load taken to puncture the rind was used as a measure of rind puncture strength.

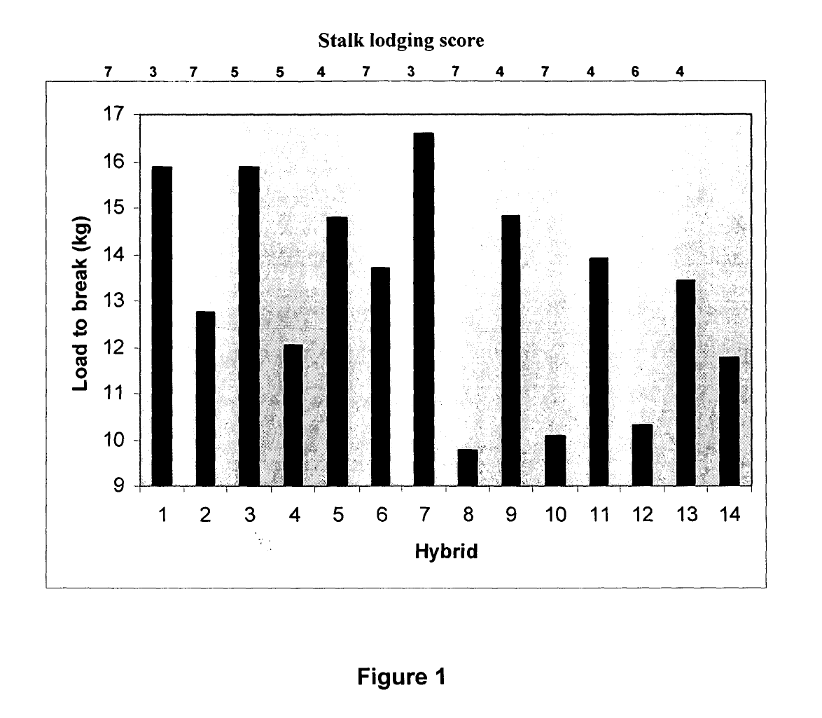

Load needed to break the internode was used as a measure of mechanical strength. The internodes are stronger toward the base of the stalk. This mechanical stalk breaking strength, or the “load to break”, was used to classify the hybrids with known stalk characteristics into respective categories based on the internodal breaking strength. The load to break the internodal zone was very similar to the lodging score that had been assigned to the hybrids based on field observations (see FIG. 1). Approximately 90% of the variation for internodal breaking strength was explained by unit stalk dry matter below the ear (47%), stalk diameter (30%), and rind puncture resistance (10%). Moisture levels above 30% in the stalk tissue masked the contribution of the rind tissue to breaking strength. The internodal breaking strength was highly correlated with the mount of cellulose per unit length of the stalk.

Four of the CesA genes were expressed under the control of a weak constitutive promoter, F3.7 (see Sean Coughlin, et al., U.S. patent application Ser. No. 09/387,720, filed Aug. 30, 1999). Table 4 discloses the construct numbers, corresponding sequence IDs from the patent, promoters, and the gene names. In2 is an inducible promoter from the In2 gene from maize. The In2 promoter responds to benzenesulfonamide herbicide safeners (see Hershey et al. (1991) Mol Gen. Genetics 227:229-237 and Gatz et al. (1994) Mol. Gen Genetics 243:32-38).

TABLE 4

| Construct | CesA SEQ ID NO. | Promoter | Gene name |
|---|---|---|---|
| 1 | 1 | F3.7 | CesA1 |
| 2 | 9 | F3.7 | CesA4 |
| 3 | 13 | F3.7 | CesA5 |
| 4 | 17 | F3.7 | CesA8 |
| 5 | Control | IN2 | GUSINT |

Twenty-five individual T₀ events for each construct were generated in a hybrid maize background using Agrobacterium-mediated transformation. Data for various traits, such as plant height, stalk mass below ear, stalk diameter, internodal breaking strength, and structural material and cellulose percentages in the internodal tissue were collected.

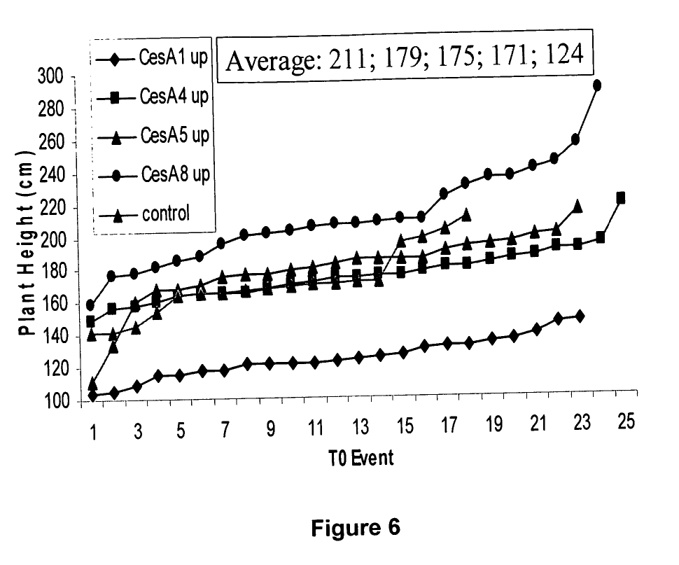

The plants from the transgenic events generated using the CesA8 gene were significantly taller in comparison to the control plants containing a GUS gene. Interstingly, a reduction in height was observed when the CesA1 gene was introduced. The other two genes, CesA4 and CesA5, did not differ from the control plants. (See FIG. 6.) It has long been known that cellulose synthase occurs as a terminal rosette complex consisting of multiple functional cellulose synthase polypeptides that are organized in a ring with a hexagonal symmetry. Each of the six members of the ring is believed to contain six or more functional enzyme units. In general, 36 or more cellulose chains are extruded simultaneous to their synthesis through the plasma membrane into the apoplast. These chains are crystallized into a microfibril right as they come in contact with each other after extrusion through the rosette complex. A functional cellulose synthase is believed to consist of two polypeptides derived form different CesA genes, forming a heterodimer, resulting in a total of 72 or more CesA polypeptides in each rosette.

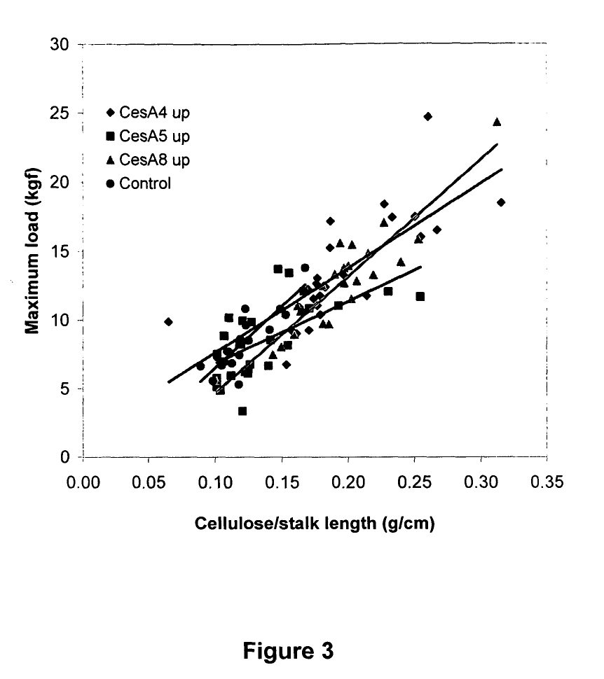

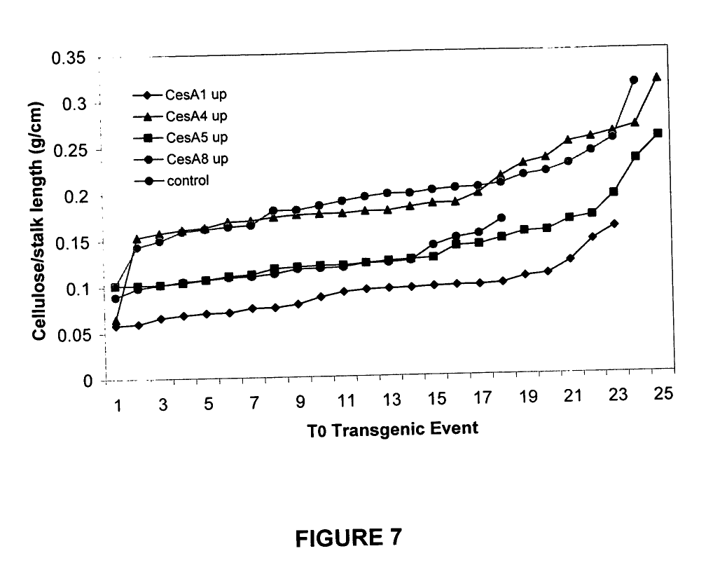

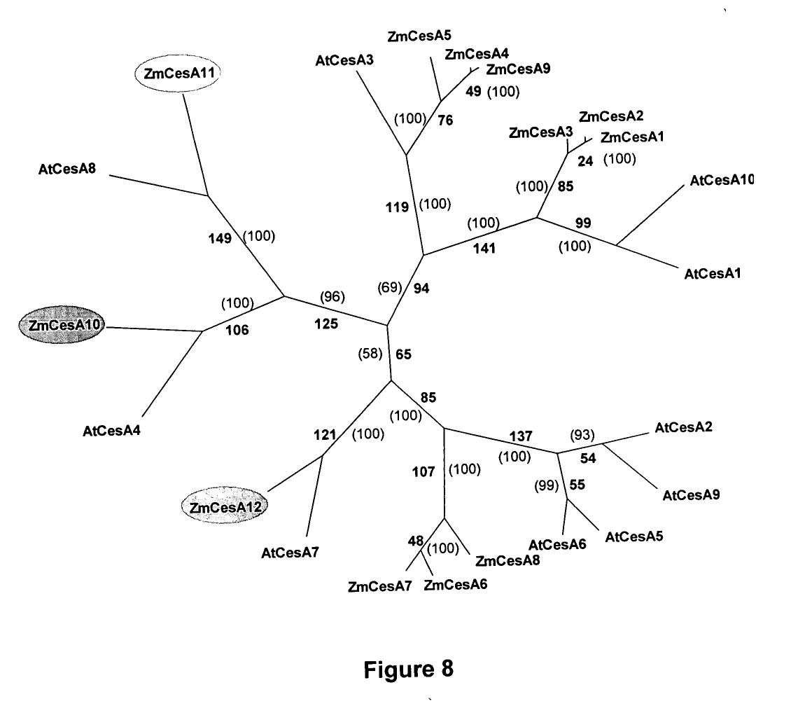

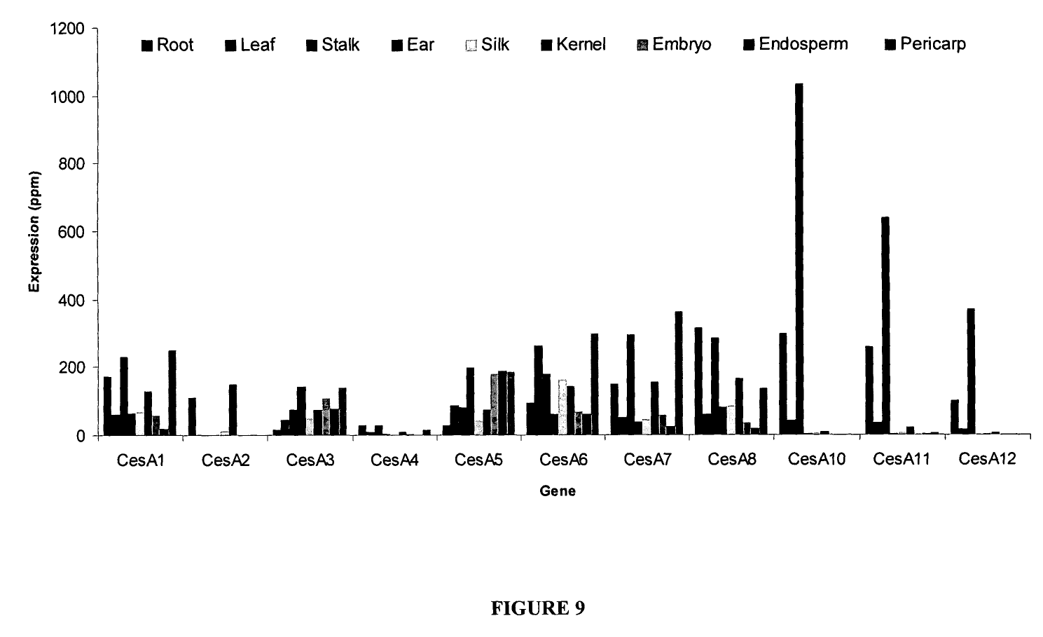

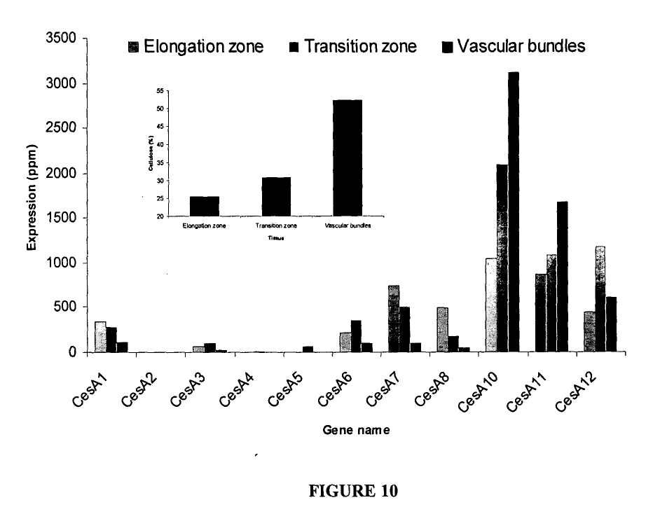

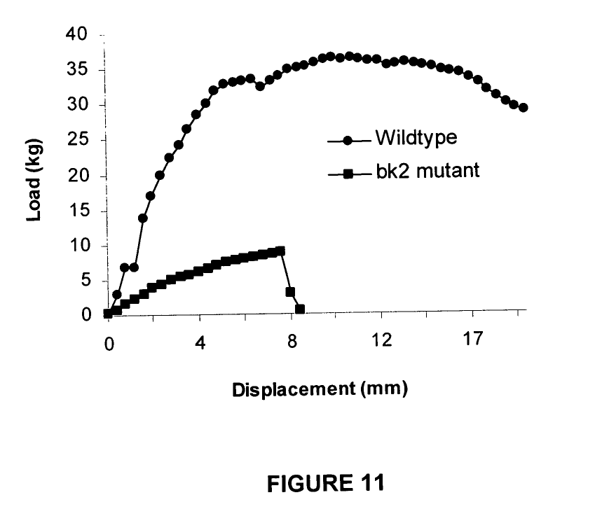

While not intending to be limited to a single theory, it is possible that a homodimer could also form a functional enzyme. Therefore, the possible reasons for a reduction in plant height in the events where CesA1 was overexpressed are: 1) the other CesA gene with which its polypeptide forms a heterodimer is down-regulated, and 2) the expression of the other gene is not affected but the CESA1 homodimer forms a nonfunctional enzyme, in which case the functional dimers are competed out of the rosette complex. In the latter case, the overexpressed gene behaves as a dominant repressor of cellulose synthesis. This should manifest in the form of microfibrils with fewer cellulose chains. This could be detected by some physical techniques such as differential scanning calorimetry (DSC). The reverse could be true for the CesA8 gene whose homodimers may be functional, and/or whose overexpression might induce the expression of its partner gene the product of which it uses to make a functional enzyme. The fact that an increase in height is observed may result from stalk becoming an active sink when CesA8 is overexpressed. Stalk is usually considered to be a passive sink which cannot compete well with the developing ear. This argument is supported by the observation that the plants containing CesA8 as a transgene had smaller ears. Cellulose content and stalk length below the ear is highly correlated with the breaking strength of the stalk (See FIG. 3). An increase in cellulose production can be accommodated by the following alterations: 1) synthesis of the other cell wall constituents stays constant, leading to an increased cellulose percentage in the wall; and 2) increase in cellulose synthesis upregulates the synthesis of the other cell wall constituents as well, in which case the percentage of cellulose does not change in the wall but the amount of cellulose in a unit length does. Two of the CesA genes, CesA4 and CesA8, showed an increase in the amount of cellulose in a unit length of the stalk below the ear (See FIG. 7). One of the genes, CesA5, did not have any effect on the amount of cellulose in the stalk. It was recently suggested, based on its expression pattern in different tissues, that CesA5 might actually be involved in the formation of some non-cellulosic polysaccharide, most probably mixed-linked glucan (Dhugga, K. S. 2001, Curr. Opin. Plant Biol., 4:488-493). The data in the accompanying figures seem to support this argument.

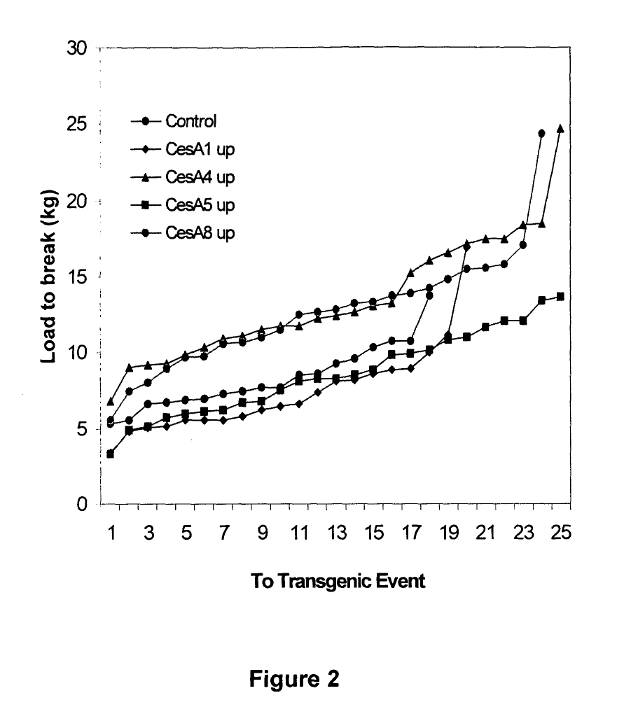

The internodes were subjected to breakage with a 3-point Instron and the load to break plotted as a function of unit cellulose amount. (See FIG. 2.) A high correlation between these two traits is observed from the multiple events, particularly for CesA8 (FIG. 3). We have found from other studies that this gene is involved in cellulose synthesis in the vascular bundles in the elongating cells (Holland, N., Holland, D., Helentjaris, T., Dhugga, K. S., Xoconostle-Cazares, and Delmer, D. P. (2000). A comparative analysis of the plant cellulose synthase (CesA) gene family. Plant Physiol. 123, 1313-1323). These data support our previous observations and supports the observation that the amount of cellulose in a unit length of stalk below the ear results in an increased stalk strength.

### EXAMPLE 11

### Characterization of the Brittle Stalk Mutant

The role of cellulose in determining mechanical strength in maize is dramatically exhibited by a spontaneous mutant, brittle stalk (bk2), which maps to chromosome 9L (Langham D. G., Brittle stalk-2 (bk2). Maize Genet. Co-Op News Lett. MNL14: 21-22, 1940). The mutant plants have substantially reduced strength in all the aboveground tissues to the extent that the tissues snap upon slight bending starting at about five-leaf stage (Coe E. H. J., Neuffer M. G., and Hoisington M. G., Corn and com improvement, G. F. Sprague, ed. (Madison: American Society of Agronomy, 1988). We measured the flexural strength of the internodes below the ear in greenhouse-grown plants that were derived from the seed obtained from one selfed ear segregating for bk2. The mutant stalks had ˜40% of the strength of the wildtype sibs (Table 5). Whereas the wildtype stalk stayed attached after breakage in a 3-point flexural test, the bk2 stalk snapped at yield point (FIG. 11). The mutant plants had significantly reduced dry matter and cellulose contents per unit length of the stalk (Table 5). The cell wall content was reduced by ˜12% in the total dry matter in the mutant stalks but cellulose concentration was reduced by approximately 30% in comparison to their wildtype sibs. The amount of cellulose per unit length of the stalk was highly correlated with the load to break (R²=0.98). The bk2 plants were grown in pots adjacent to the wildtype plants in controlled conditions and were similar in stalk diameter and ear height (Table 5). This indicates that the reduction in dry matter and cellulose contents per unit length explained the differences for stalk strength between the mutant and the wildtype plants. Our results are in agreement with the previous studies in barley where only the cellulose content was found to be affected in the straw of the brittle culm mutant (Kokubo A., Kuraishi S.,and Sakurai N. Plant Physiol. 91: 876-882, 1989, and Kokubo A., Sakurai N., Kuraishi S., and Takeda K., Plant Physiol. 97: 509-514, 1991.) The number of cellulose molecules, not the molecular mass, appeared to be reduced in the mutant barley plants, which may have resulted from a reduction in the number of the cellulose synthase complexes in the plasma membrane (Kokubo et al., 1991, supra).

An apparent increase in lignin concentration in the wall fraction of the bk2 stalks can be explained by the fact that it is expressed as a fraction of total dry matter and a reduction in one of the constituents would be expected to result in a corresponding increase in the remaining ones (Table 5). Actually, whereas lignin in the bk2 stalks showed an apparent increase of 13% over their wildtype sibs, the rest of the cell wall (mostly hemicellulose with some protein and ash) content increased by approximately 20%. Since cellulose constitutes a major sink in the stalk tissue by being the most abundant constituent, a limitation in its synthesis could reduce the formation of the rest of the cell wall, thereby causing an overall reduction in dry matter deposition in the plant.

TABLE 5

| Trait | Wildtype | bk2 |
|---|---|---|
| Ear height (cm) | 102.00 ± 8.8 | 106.33 ± 11.8 |
| Stalk diameter (mm) | 23.84 ± 0.27 | 23.40 ± 0.46 |
| Stalk dry mass (g) | 89.43 ± 3.39 | 62.08 ± 8.46 |
| Moisture (%) | 79.20 ± 0.21 | 84.87 ± 1.04 |
| Dry matter (g/cm) | 0.68 ± 0.04 | 0.43 ± 0.07 |
| Displacement to break (mm) | 11.83 ± 0.46 | 6.51 ± 1.10 |
| Load to break (kg) | 23.68 ± 2.25 | 9.04 ± 2.66 |
| Insoluble dry matter (%) | 51.57 ± 1.00 | 45.20 ± 1.69 |
| Cellulose (%) | 33.30 ± 0.56 | 23.76 ± 0.68 |
| Lignin (%) | 9.07 ± 0.21 | 10.28 ± 0.63 |
| Remainder cell wall (%) | 9.20 ± 1.68 | 11.16 ± 2.03 |
| Cellulose (g/cm) | 0.24 ± 0.114 | 0.11 ± 0.019 |

Characteristics of the stalk tissue of the brittle stalk (bk2) mutant of maize. Three greenhouse-grown plants from each of the bk2 and its wildtype sib, both derived from seeds obtained from the same selfed ear were evaluated for different traits approximately two weeks after flowering. Total dry matter was measured in the stalk portion below the ear node. Three internodes below the ear node were subjected to a 3-point flexural test. Structural dry matter and cellulose contents were determined in duplicates on each of the three plants from the 3^{rd} internode below the ear node. Since only the cellulose content is reduced in the wall of the maize bk2 stalks and brittle culm straws of barley, our initial hypothesis was that Bk2 might be a CesA gene. Mutated form of each of the three different secondary wall forming CesA genes from Arabidopsis, named Irx for irregular xylem, results in diminished cellulose formation and reduced stem strength (Taylor N. G. et al., 1999. Plant Cell 11: 769-779, 1999.) However, none of the twelve CesA genes from maize maps to chromosome 9 on which the Bk2 gene is located (Holland N., et al., Plant Physiol. 123: 1313-1323, 2000). Nor does the mutant form of Sus1, which maps very close to Bk2, exhibit a brittle phenotype. At least some members of two of the other gene families that might alter cellulose formation, i.e., uridine diphosphate glucose pyrophosphorylase (UGPase) and membrane-associated β-1, 4-glucanase, are excluded as candidates for Bk2 because they map to other locations (Helentjaris and Rafalski, personal communications). The Bk2 gene might encode a transacting factor that adversely affects the formation of cellulose, particularly in the secondary wall. This could result from a suppression of the CesA gene or that of other members of the cellulose synthase complex. Alternatively, Bk2 could be involved in wall assembly but is not a β-1, 4-glucanase. The bk2 plants looked phenotypically normal suggesting that most likely this gene affects secondary wall formation because plants harboring mutations in the genes that affect primary wall formation exhibit severe phenotypic alterations (Arioli T. et al., Science 279: 717-720,1998; and Fagard M. et al., Plant Cell, 12:2409-2423. 2000.

### EXAMPLE 12

### Cellulose Synthase Gene Family

Deduced protein sequences for the Arabidopsis CESA proteins were downloaded from the web site at cellwall.stanford.edu. Maize sequences for the genes CesA1-9 are available in GenBank (Holland N., et al., Plant Physiol. 123: 1313-1323, 2000). Sequence alignment was carried out using CLUSTAL W program (Thompson J. D. et al., Nucleic Acids Res. 22: 4673-4680, 1994). Parsimony and neighbor-joining analyses were performed using the PAUP program (Swofford D. L., PAUP*: Phylogenetic analysis using parsimony (and other methods), Volume Version 4 (Sunderland, Mass.: Sinauer Associates), 1998). To assess the degree of support for each branch on the tree, bootstrap analysis with 500 replicates was performed (Felsenstein J., Evolution 39: 783-791, 1985). Maximum-likelihood tree was also reconstructed using proML algorithm implemented in the PHYLIP package by J. Felsenstein (Phylogeny Inference Package, version 3.6a2.1; web site at evolution.genetics.washington.edu/phylip.html). Both neighbor-joining and maximum-likelihood trees showed very similar tree topologies as that of the maximally parsimonious tree with minor terminal branch differences.

A previous report described nine of the CesA genes from maize that were isolated from Pioneer proprietary genomics EST database (Holland N., et al., Plant Physiol. 123: 1313-1323, 2000). Based on their similarity to the known CesA genes from Arabidopsis and other species at the time, genes were classified into two categories, i.e., primary and potentially secondary wall forming ones. Because of the proximity of the lade consisting of CesA6, 7, and 8 genes to the secondary wall forming genes from other species and the expression pattern of CesA8, these genes may be involved in secondary wall formation (Holland N., et al., Plant Physiol. 123: 1313-1323, 2000). None of those genes, however, formed a true lade with any of the secondary wall forming genes from other plant species.

Genes involved in secondary wall formation are usually underrepresented in the EST databases as the latter are generally enriched in sequences derived from immature tissues. These genes are expected to be preferentially expressed in the transition zone of the stalk where the cells in the vascular bundles start depositing secondary wall at a higher rate. cDNA libraries were constructed from the elongation and transition zones of an elongating maize internode and obtained ˜8000 ESTs from each of the libraries. Three additional full-length genes were designated as CesA10, CesA11, and CesA12 were isolated from among these ESTs. This approach is similar to the one used to isolate the first plant CesA gene from only a few hundred ESTs derived from the developing cotton fibers during peak secondary wall formation (Pear J. R. et al., Proc. Natl. Acad. Sci. USA 93: 12637-12642, 1996). The CesA genes are generally expressed at a low level as judged from their occurrence in the EST databases and from gene expression studies but can occur at higher frequencies in specific tissues as shown later in the gene expression profiling section (Dhugga K. S., Curr. Opin. Plant Biol. 4: 488-493, 2001).

An unrooted cladogram comprising the maize and Arabidopsis CESA proteins is shown in FIG. 4. The deduced amino acid sequences of the three additional maize CesA genes cluster with the corresponding deduced proteins from Arabidopsis where they are known to be involved in secondary wall formation. ZmCESA10, ZmCESA11, and ZmCESA12 group with AtCESA4 (IRX5), AtCESA8 (IRX1), and AtCESA7 (IRX3), respectively. This suggests that the different subclasses of the CesA genes diverged early in evolution, at least before the separation of monocots and dicots (Holland N., et al., Plant Physiol. 123: 1313-1323, 2000). Each of the Irx genes is expressed in the same cell type in the vascular tissue in Arabidopsis (Taylor N.G.et al., Proc. Natl. Acad. Sci. USA 100: 1450-1455, 2003). Phylogenetic clustering of the maize CESA proteins with the IRX proteins from Arabidopsis and their highest expression in the transition zone of the internode suggest that these genes are involved in secondary wall formation. Gene expression profiling studies described in the following section lend further support to this suggestion.

### EXAMPLE 13

Expression Profiling of the CesA Gene Family

FIG. 5 depicts the expression pattern of the maize CesA genes in different tissues as studied using the massively parallel signature sequencing (MPSS) technology (Brenner S. et al., Nature Biotechnology 18: 630-634, 2000; Brenner S. et al., Proc. Natl. Acad. Sci. USA 97: 1665-1670, 2000; Hoth S. et al., J. Cell Sci. 115: 4891-4900, 2002; and, Meyers B. C. et al., Plant J. 32: 77-92, 2002). Similar data from a smaller set of libraries were presented in a previous report (Dhugga K. S., Curr. Opin. Plant Biol. 4: 488-493, 2001). The current data are summarized from 76 different libraries whereas the previous data were derived from only 23 libraries. Two general conclusions can be drawn from these data: 1) CesA genes 1-8, with the exception of CesA2, are expressed at different levels in a majority of the tissues and 2) CesA10-12 are selectively expressed in those tissues that are rich in secondary wall. For CesA1-8, these data are in overall agreement with the previous data with the exception of CesA2, which, after reanalysis is found to be expressed only in the root and the kernel tissues and at a very low level in the silk tissue (Dhugga K. S., Curr. Opin. Plant Biol. 4: 488-493, 2001). CesA5 and CesA6 are the highest expressed CesA genes in the endosperm and leaf tissues, respectively. CesA10, CesA11, and CesA12 are most highly expressed in the stalk tissue although CesA7 and CesA8 approach the level of expression of CesA12. The expression of none of the CesA genes is detected in the mature pollen grain.

Theoretically, the whole expressed genome is analyzed by the MPSS technology each time a library is screened for unique tags (Brenner S. et al., Nature Biotechnology 18: 630-634, 2000; and, Brenner S. et al., Proc. Natl. Acad. Sci. USA 97: 1665-1670, 2000). Quantitative measures of the expression levels of different gene tags in the MPSS, as opposed to the ratios across paired tissues or treatments in the microarray-based platforms, combined with the depth of signature sequencing (>1 million) for each of the libraries make it possible to compare gene expression patterns across multiple, independent experiments. A correlation coefficient matrix showing the relationship for the expression pattern among the maize CesA genes is shown in Table 2. As reported previously, CesA1 is expressed to a large degree in similar tissues as CesA7 (R²=0.49) and CesA8 (R²=0.40) (Dhugga, 2001). CesA2 as discussed above is expressed only a in a few tissues and its expression pattern does not appreciably correlate with any of the other genes. A lack of appreciable correlation of CesA3, CesA5, or CesA6 with any of the genes suggests that the relatively high correlation coefficients observed among some of the gene pairs may have biological relevance. Dimerization of the CESA proteins has been proposed for the formation of a functional cellulose synthase complex (Doblin, M. S. et al., Plant Cell Physiol. 43:1407-1420, 2002; Kurek I. et al., Proc. Natl. Acad. Sci. USA 99: 11109-11114, 2002; and, Scheible W. R. et al., Proc. Natl. Acad. Sci. USA 98:10079-10084, 2001). All three of the secondary wall forming CESA proteins (IRX1, IRX3, and IRX5) have been reported to be involved in the formation of a functional cellulose synthase complex (Taylor N. G. et al., Proc. Natl. Acad. Sci. USA 100: 1450-1455, 2003). Theoretically, only two catalytic subunits are needed to provide juxtaposed catalytic sites for the formation of a β-bond without having to rotate the chain after each bond formation (Dhugga K. S., Curr. Opin. Plant Biol. 4: 488-493, 2001). This could be accomplished either by a homo- or a heterodimer. It is possible, however, that multiple CESA proteins are needed to form a functional rosette (Taylor N. G.et al., Proc. Natl. Acad. Sci. USA 100: 1450-1455, 2003). Relatively high correlation coefficients among the expression levels of CesA1, CesA7, and CesA8 genes suggest that their proteins might form a functional complex in certain cell types.

For the CesA10, CesA11, and CesA12 genes, the correlation coefficients are around 0.9 among different pairs, indicating that these genes are mostly coexpressed. Since these genes were isolated from the tissues where secondary wall is actively deposited, i.e., the transition zone of an elongating internode, we subjected the elongation zone, transition zone, and the isolated vascular bundles to MPSS analysis (FIG. 10). Of all the CesA genes, CesA10, CesA11 and CesA12 are most highly expressed in the internodal tissue as well as in the isolated vascular bundles. The inset in FIG. 10 shows cellulose concentration in the elongation and transition zones and the vascular bundles from an elongating internode. The expression pattern of two of the genes, CesA10 and CesA11, parallels the cellulose concentration in these three tissues. CesA12, however, is expressed at a lower level than expected in the vascular bundles if its protein were to form a functional complex with the proteins of CesA10 and CesA11.

None of the remaining eight CesA genes is significantly expressed in the vascular bundles (FIG. 10). In general, the expression of these genes is highest in the elongation zone of the stalk tissue. Low-level expression of these genes in the vascular bundle library could be real but could also be because of the contaminating parenchymatous cells. CesA1, CesA6, CesA7, and CesA8 may be responsible for making cellulose in the non-vascular, ground tissue cells in the maize stalk.

As implied by the correlation coefficients among the expression patterns of these genes, CesA10 and CesA11 seem to have nearly identical expression patterns across the three tissues examined in detail, whereas CesA12 is expressed at a lower level in the vascular bundles than in the total tissue from the transition zone (FIG. 10). CESA12 is also evolutionarily more distant from both CESA10 and CESA11 than the latter two are from each other and is actually closer to CESA6, 7, and 8 (FIG. 4). This implies that CesA12, in addition to its expression in the vascular bundles, is also expressed in the non-vascular cells. Since it is expressed at a higher level in the transition zone than the elongation zone or any other tissue, it is most likely expressed in the sclerenchymatous cells surrounding the vascular bundles in the hypodermis. This supports the hypothesis that, at least for secondary wall formation, only two different coexpressed genes may be needed in the same cell type for the formation of a functional cellulose synthase complex.

The above examples are provided to illustrate the invention but not to limit its scope. Other variants of the invention will be readily apparent to one of ordinary skill in the art and are encompassed by the appended claims. All publications, patents, patent applications, and computer programs cited herein are hereby incorporated by reference.

What is claimed is:

1. An isolated nucleic acid comprising a member selected from the group consisting of:
(a) a polynucleotide having at least 70% sequence identity, as determined by the GAP algorithm under default parameters, to a polynucleotide selected from the group consisting of SEQ ID NOS: 25, 27 and 29;
(b) a polynucleotide encoding a polypeptide selected from the group consisting of SEQ ID NOS: 26, 28 and 30;
(c) a polynucleotide amplified from a Zea mays nucleic acid library using primers which selectively hybridize, under stringent hybridization conditions, to loci within a polynucleotide selected from the group consisting of SEQ ID NOS: 25, 27 and 29;
(d) a polynucleotide which selectively hybridizes, under stringent hybridization conditions and a wash in 0.1×SSC at 65° C., to a polynucleotide selected from the group consisting of SEQ ID NOS: 25, 27 and 29;
(e) a polynucleotide selected from the group consisting of SEQ ID NOS: 25, 27 and 29;
(f) a polynucleotide which is complementary to a polynucleotide of (a), (b), (c), (d), or (e); and
(g) a polynucleotide comprising at least 25 contiguous nucleotides from a polynucleotide of (a), (b), (d), (e), or (f).

2. A recombinant expression cassette, comprising a member of claim 1 operably linked, in sense or anti-sense orientation, to a promoter.

3. A host cell comprising the recombinant expression cassette of claim 2.

4. A transgenic plant comprising the recombinant expression cassette of claim 2.

5. The transgenic plant of claim 4, wherein said plant is a monocot.

6. The transgenic plant of claim 4, wherein said plant is a dicot.

7. The transgenic plant of claim 4, wherein said plant is selected from the group consisting of: maize, soybean, sunflower, sorghum, canola, wheat, alfalfa, cotton, rice, barley, millet, peanut, and cocoa.

8. A seed from the transgenic plant of claim 4.

9. A method of modulating the level of cellulose synthase in a plant cell, comprising:
(a) introducing into a plant cell a recombinant expression cassette comprising a polynucleotide of claim 1 operably linked to a promoter;
(b) culturing the plant cell under plant cell growing conditions; and
(c) expressing said polynucleotide for a time sufficient to modulate the level of cellulose synthase in said plant cell.

10. The method of claim 9, wherein the plant cell is is selected from the group consisting of: maize, soybean, sunflower, sorghum, canola, wheat, alfalfa, cotton, rice, barley, millet, peanut, and cocoa.

11. A method of modulating the level of cellulose synthase in a plant, comprising:
(a) introducing into a plant cell a recombinant expression cassette comprising a polynucleotide of claim 1 operably linked to a promoter;
(b) culturing the plant cell under plant cell growing conditions;
(c) regenerating a plant from said plant cell; and
(d) expressing said polynucleotide for a time sufficient to modulate the level of cellulose synthase in said plant.

12. The method of claim 11, wherein the plant is selected from the group consisting of: maize, soybean, sunflower, sorghum, canola, wheat, alfalfa, cotton, rice, barley, millet, peanut, and cocoa.

13. An isolated protein comprising a member selected from the group consisting of:
(a) a polypeptide of at least 20 contiguous amino acids from a polypeptide selected from the group consisting of SEQ ID NOS: 26, 28 and 30;
(b) a polypeptide selected from the group consisting of SEQ ID NOS: 26, 28 and 30;
(c) a polypeptide having at least 70% sequence identity to, and having at least one epitope in common with, a polypeptide selected from the group consisting of SEQ ID NOS: 26, 28 and 30, wherein said sequence identity is determined by the GAP algorithm under default parameters; and,
(d) at least one polypeptide encoded by a member of claim 1.

14. A method of modifying expression of a cellulose synthase gene in a maize plant, comprising:
(a) identifying, from a population of maize plants mutagenized with the Mu transposable element, those plants containing one or more Mu insertions within a polynucleotide of claim 1;
(b) selecting those plants showing modified cellulose syse gene expression.

15. The method of claim 14, where expression of the cellulose synthase gene is down-regulated.


SEQUENCE LISTING (52 sequences)

SEQ ID NO: 1 - DNA, 3780 bp, Zea mays
gtcgacccacgcgtccgcagcagcagaagcactgcgcggcattgcagcgatcgagcgggaggaatttggggcatggtggtcgccaacgccgctcggatctagaggcccgcacgggccgattggtctccgcccgcctcgtcggtgttggtgtcgttggcgtgtggagccgtctcggtgggagcagcggggagggagcggagatggcggccaacaaggggatggtggcgggctcgcacaaccgcaacgagttcgtcatgatccgccacgacggcgatgtgccgggctcggctaagcccacaaagagtgcgaatggacaggtctgccagatttgcggtgactctgtgggtgtttcagccactggtgatgtctttgttgcctgcaatgagtgtgccttccctgtctgccgcccatgctatgagtatgagcgcaaggaggggaaccaatgctgcccccagtgcaagactagatacaagagacagaaaggtagccctcgagttcatggtgatgaggatgaggaagatgttgatgacctagacaatgaattcaactacaagcaaggcagtgggaaaggcccagagtggcaactgcaaggagatgatgctgatctgtcttcatctgctcgccatgagccacatcatcggattccacgcctgacaagcggtcaacagatatctggagagattcctgatgcttcccctgaccgtcattctatccgcagtccaacatcgagctatgttgatccaagcgtcccagttcctgtgaggattgtggacccctcgaaggacttgaattcctatgggcttaatagtgttgactggaaggaaagagttgagagctggagggttaaacaggacaaaaatatgatgcaagtgactaataaatatccagaggctagaggaggagacatggaggggactggctcaaatggagaagatatgcaaatggttgatgatgcacggctacctttgagccgtatcgtgccaatttcctcaaaccagctcaacctttaccgggtagtgatcattctccgtcttatcatcctgtgcttcttcttccagtatcgtgtcagtcatccagtgcgtgatgcttatggattatggctagtatctgttatctgcgaggtctggtttgccttgtcttggcttctagatcagttcccaaaatggtatccaatcaaccgtgagacatatcttgacaggcttgcattgaggtatgatagagagggagagccatcacagctggctcccattgatgtcttcgtcagtacagtggatccattgaaggaacctccactgatcacagccaacactgttttgtccattctttctgtggattaccctgttgacaaagtgtcatgctatgtttctgatgatggttcagctatgctgacttttgagtctctctcagaaaccgcagaatttgctagaaagtgggttcccttttgtaagaagcacaatattgaaccaagagctccagaattttactttgctcaaaaaatagattacctgaaggacaaaattcaaccttcatttgttaaggaaagacgcgcaatgaagagggagtatgaagaattcaaagtaagaatcaatgcccttgttgccaaagcacagaaagtgcctgaagaggggtggaccatggctgatggaactgcatggcctgggaataatcctagggaccatcctggcatgattcaggttttcttggggcacagtggtgggctcgacactgatggaaatgagttaccacgtcttgtctatgtctctcgtgaaaagagaccaggctttcagcatcacaagaaggctggtgcaatgaatgcgctgattcgtgtatctgctgtgctgacaaatggtgcctatcttctcaatgtggattgcgaccattacttcaatagcagcaaagctcttagagaagcaatgtgcttcatgatggatccggctctaggaaggaaaacttgttatgtacaatttccacagagatttgatggcattgacttgcacgatcgatatgctaatcggaacatagttttctttgatatcaacatgaaaggtctggatggcattcagggtccagtttacgtgggaacaggatgctgtttcaatagacaggctttgtatggatacgatcctgttttgactgaagctgatctggagccaaacattgttattaagagctgctgtggtagaaggaagaaaaagaacaagagttatatggatagtcaaagccgtattatgaagagaacagaatcttcagctcccatcttcaatatggaagacatcgaagagggtattgaaggttacgaggatgaaaggtcagtgcttatgtcccagaggaaattggagaaacgctttggtcagtctcctattttcattgcatccacctttatgacacaaggtggcataccaccttcaacaaacccagcttctctactaaaggaagctatccatgtcatcagttgtggatatgaggacaaaactgaatggggaaaagagattggctggatctatggttcagtaacggaggatattctgactgggtttaaaatgcatgcaaggggctggcaatcaatctactgcatgccaccacgaccttgtttcaagggttctgcaccaatcaatctttccgatcgtcttaatcaggtgctccgttgggctcttgggtcagtggaaattctgcttagtagacattgtcctatctggtatggttacaatggacgattgaagcttttggagaggctggcttacatcaacactattgtatatccaatcacatccattccgcttattgcctattgtgtgcttcccgctatctgcctccttaccaataaatttatcattcctgagattagcaattatgctgggatgttcttcattcttcttttcgcctccatttttgccactggtatattggagcttagatggagtggtgttggcattgaagattggtggagaaatgagcagttttgggttattggtggcacctctgcccatctcttcgcagtgttccagggtctgctgaaagtgttggctgggattgataccaacttcacagttacctcaaaggcatctgatgaggatggcgactttgctgagctatatgtgttcaagtggaccagtttgctcattcctccgaccactgttcttgtcattaacctggtcggaatggtggcaggaatttcttatgccattaacagtggctaccaatcctggggtccgctctttggaaagctgttcttctcgatctgggtgatcctccatctctaccccttcctcaagggtctcatgggaaggcagaaccgcacaccaacaatcgtcattgtctggtccatccttcttgcatctatcttctccttgctgtgggtgaagatcgatcctttcatctccccgacacagaaagctgctgccttggggcaatgtggcgtcaactgctgatcgagacagtgactcttatttgaagaggctcaatcaagatctgccccctcgtgtaaatacctgaggaggctagatgggaattccttttgttgtaggtgaggatggatttgcatctaagttatgcctctgttcattagcttcttccgtgccggtgctgctgcggactaagaatcacggagcctttctaccttccatgtagcgccagccagcagcgtaagatgtgaattttgaagttttgttatgcgtgcagtttattgttttagagtaaattatcatttgtttgtgggaactgttcacacgagcttataatggcaatgctgttatttaaaaaaaaaaaaaaaagggcggccgc

SEQ ID NO: 2 - protein, 1075 aa, Zea mays
MAANKGMVAGSHNRNEFVMIRHDGDVPGSAKPTKSANGQVCQICGDSVGVSATGDVFVACNECAFPVCRPCYEYERKEGNQCCPQCKTRYKRQKGSPRVHGDEDEEDVDDLDNEFNYKQGSGKGPEWQLQGDDADLSSSARHEPHHRIPRLTSGQQISGEIPDASPDRHSIRSPTSSYVDPSVPVPVRIVDPSKDLNSYGLNSVDWKERVESWRVKQDKNMMQVTNKYPEARGGDMEGTGSNGEDMQMVDDARLPLSRIVPISSNQLNLYRVVIILRLIILCFFFQYRVSHPVRDAYGLWLVSVICEVWFALSWLLDQFPKWYPINRETYLDRLALRYDREGEPSQLAPIDVFVSTVDPLKEPPLITANTVLSILSVDYPVDKVSCYVSDDGSAMLTFESLSETAEFARKWVPFCKKHNIEPRAPEFYFAQKIDYLKDKIQPSFVKERRAMKREYEEFKVRINALVAKAQKVPEEGWTMADGTAWPGNNPRDHPGMIQVFLGHSGGLDTDGNELPRLVYVSREKRPGFQHHKKAGAMNALIRVSAVLTNGAYLLNVDCDHYFNSSKALREAMCFMMDPALGRKTCYVQFPQRFDGIDLHDRYANRNIVFFDINMKGLDGIQGPVYVGTGCCFNRQALYGYDPVLTEADLEPNIVIKSCCGRRKKKNKSYMDSQSRIMKRTESSAPIFNMEDIEEGIEGYEDERSVLMSQRKLEKRFGQSPIFIASTFMTQGGIPPSTNPASLLKEAIHVISCGYEDKTEWGKEIGWIYGSVTEDILTGFKMHARGWQSIYCMPPRPCFKGSAPINLSDRLNQVLRWALGSVEILLSRHCPIWYGYNGRLKLLERLAYINTIVYPITSIPLIAYCVLPAICLLTNKFIIPEISNYAGMFFILLFASIFATGILELRWSGVGIEDWWRNEQFWVIGGTSAHLFAVFQGLLKVLAGIDTNFTVTSKASDEDGDFAELYVFKWTSLLIPPTTVLVINLVGMVAGISYAINSGYQSWGPLFGKLFFSIWVILHLYPFLKGLMGRQNRTPTIVIVWSILLASIFSLLWVKIDPFISPTQKAAALGQCGVNC

SEQ ID NO: 3 - DNA, 25 bp, Zea mays
atggcggccaacaaggggatggtgg

SEQ ID NO: 4 - DNA, 25 bp, Zea mays
tcagcagttgacgccacattgcccc

SEQ ID NO: 5 - DNA, 2830 bp, Zea mays
  misc_feature 2809, 2818, 2824, 2826, 2829: n = A,T,C or G
tacctctaagtcgcatagttccgatatctccaaacgagcttaacctttatcggatcgtgattgttctccggcttatcatcctatgtttcttctttcaatatcgtataactcatccagtggaagatgcttatgggttgtggcttgtatctgttatttgtgaagtttggtttgccttgtcttggcttctagatcagttcccaaagtggtatcctatcaaccgtgaaacttacctcgatagacttgcattgagatatgatagggagggtgagccatcccagttggctccaatcgatgtctttgttagtacagtggatccacttaaggaacctcctctaattactggcaacactgtcctgtccattcttgctgtggattaccctgttgacaaagtatcatgttatgtttctgatgacggttcagctatgttgacttttgaagcgctatctgaaaccgcagagtttgcaaggaaatgggttcccttttgcaagaaacacaatattgaacctagggctccagagttttactttgctcgaaagatagattacctaaaggacaaaatacaaccttcttttgtgaaagaaaggcgggctatgaagagggagtgtgaagagttcaaagtacggatcgatgcccttgttgcaaaagcgcaaaaaatacctgaggagggctggaccatggctgatggcactccttggcctgggaataaccctagagatcatccaggaatgatccaagtattcttgggccacagtggtgggcttgacacggatgggaatgagttgccacggcttgtttatgtttctcgtgaaaagaggccaggcttccagcaccacaagaaggctggtgccatgaatgctttgattcgcgtatcagctgtcctgacgaatggtgcttatcttcttaatgtggattgtgatcactacttcaatagcagcaaagctcttagagaggctatgtgtttcatgatggatccagcactaggaaggaaaacttgctatgttcagtttccacaaagatttgatggtatagacttgcatgatcgatatgcaaaccggaacattgtcttctttgatattaatatgaagggtctagatggcattcaaggacctgtttatgtgggaacaggatgctgtttcaataggcaggccttgtatggctatgatcctgtattgacagaagctgatttggagcctaacattatcattaaaagttgctgtggcggaagaaaaaagaaggacaagagctatattgattccaaaaaccgtgatatgaagagaacagaatcttcggctcccatcttcaacatggaagatatagaagagggatttgaaggttacgaggatgaaaggtcactgcttatgtctcagaagagcttggagaaacgctttggccagtctccaatttttattgcatccacctttatgactcaaggtggcatacccccttcaacaaacccaggttccctgctaaaggaagctatacatgtcattagttgtggatatgaggataaaacagaatgggggaaagagatcggatggatatatggctctgttactgaagatattttaactggtttcaagatgcatgcaagaggttggatatccatctactgcatgccacttcggccttgcttcaagggttctgctccaattaatctttctgatcgtctcaaccaagtgttacgctgggctcttggttcagttgaaattctacttagcagacactgtcctatctggtatggttacaatggaaggctaaagcttctggagagactggcatacatcaacaccattgtttatccaattacatctatcccactagtagcatactgcgtccttcctgctatctgtttactcaccaacaaatttattattcctgcgattagcaattatgctggggcgttcttcatcctgctttttgcttccatcttcgccactggtattttggagcttcgatggagtggtgttggcattgaggattggtggagaaatgagcagttttgggtcattggtggcacctctgcacatctctttgctgtgttccaaggtctcttaaaagtgctagcagggatcgacacaaacttcacggtcacatcaaaggcaaccgatgatgatggtgattttgctgagctgtatgtgttcaagtggacaactcttctgatcccccccaccactgtgcttgtgattaacctggttggtatagtggctggagtgtcgtatgctatcaacagtggctaccaatcatggggtccactattcgggaagctgttctttgcaatctgggtgatcctccacctctaccctttcctgaagggtctcatggggaagcagaaccgcacaccgaccatcgtcatcgtttggtccgtccttcttgcttccatattctcgctgctgtgggtgaagatcgaccccttcatatcccctacccagaaggctctttcccgtgggcagtgtggtgtaaactgctgaaatgatccgaactgcctgctgaataacattgctccggcacaatcatgatctaccccttcgtgtaaataccagaggttaggcaagacttttcttggtaggtggcgaagatgtgtcgtttaagttcactctactgcatttggggtgggcagcatgaaactttgtcaacttatgtcgtgctacttatttgtagctaagtagcagtaagtagtgcctgtttcatgttgactgtcgtgactacctgttcaccgtgggctctggactgtcgtgatgtaacctgtatgttggaacttcaagtactgattgagctgtttggtcaatgacattgagggattctctctctngaaattaanacaaantnggnt

SEQ ID NO: 6 - protein, 821 aa, Zea mays
PLSRIVPISPNELNLYRIVIVLRLIILCFFFQYRITHPVEDAYGLWLVSVICEVWFALSWLLDQFPKWYPINRETYLDRLALRYDREGEPSQLAPIDVFVSTVDPLKEPPLITGNTVLSILAVDYPVDKVSCYVSDDGSAMLTFEALSETAEFARKWVPFCKKHNIEPRAPEFYFARKIDYLKDKIQPSFVKERRAMKRECEEFKVRIDALVAKAQKIPEEGWTMADGTPWPGNNPRDHPGMIQVFLGHSGGLDTDGNELPRLVYVSREKRPGFQHHKKAGAMNALIRVSAVLTNGAYLLNVDCDHYFNSSKALREAMCFMMDPALGRKTCYVQFPQRFDGIDLHDRYANRNIVFFDINMKGLDGIQGPVYVGTGCCFNRQALYGYDPVLTEADLEPNIIIKSCCGGRKKKDKSYIDSKNRDMKRTESSAPIFNMEDIEEGFEGYEDERSLLMSQKSLEKRFGQSPIFIASTFMTQGGIPPSTNPGSLLKEAIHVISCGYEDKTEWGKEIGWIYGSVTEDILTGFKMHARGWISIYCMPLRPCFKGSAPINLSDRLNQVLRWALGSVEILLSRHCPIWYGYNGRLKLLERLAYINTIVYPITSIPLVAYCVLPAICLLTNKFIIPAISNYAGAFFILLFASIFATGILELRWSGVGIEDWWRNEQFWVIGGTSAHLFAVFQGLLKVLAGIDTNFTVTSKATDDDGDFAELYVFKWTTLLIPPTTVLVINLVGIVAGVSYAINSGYQSWGPLFGKLFFAIWVILHLYPFLKGLMGKQNRTPTIVIVWSVLLASIFSLLWVKIDPFISPTQKALSRGQCGVNC

SEQ ID NO: 7 - DNA, 25 bp, Zea mays
cctctaagtcgcatagttccgatat

SEQ ID NO: 8 - DNA, 25 bp, Zea mays
tcagcagtttacaccacactgccca

SEQ ID NO: 9 - DNA, 3773 bp, Zea mays
gtcgacccacgcgtccgctaggatcaaaaccgtctcgccgctgcaataatcttttgtcaattcttaatccctcgcgtcgacagcgacagcggaaccaactcacgttgccgcggcttcctccatcggtgcggtgccctgtccttttctctcgtccctcctccccccgtatagttaagccccgccccgctactactactactagcagcagcagcgctctcgcagcgggagatgcggtgttgatccgtgccccgctcggatctcgggactggtgccggctctgcccaggccccaggctccaggccagctccctcgacgtttctcggcgagctcgcttgccatggagggcgacgcggacggcgtgaagtcggggaggcgcggtggcggacaggtgtgccagatctgcggcgacggcgtgggcaccacggcggagggggacgtcttcgccgcctgcgacgtctgcgggtttccggtgtgccgcccctgctacgagtacgagcgcaaggacggcacgcaggcgtgcccccagtgcaagaccaagtacaagcgccacaaggggagcccggcgatccgtggggaggaaggagacgacactgatgccgatagcgacttcaattaccttgcatctggcaatgaggaccagaagcagaagattgccgacagaatgcgcagctggcgcatgaacgttgggggcagcggggatgttggtcgccccaagtatgacagtggcgagatcgggcttaccaagtatgacagtggcgagattcctcggggatacatcccatcagtcactaacagccagatctcaggagaaatccctggtgcttcccctgaccatcatatgatgtccccaactgggaacattggcaagcgtgctccatttccctatgtgaaccattcgccaaatccgtcaagggagttctctggtagcattgggaatgttgcctggaaagagagggttgatggctggaaaatgaagcaggacaaggggacgattcccatgacgaatggcacaagcattgctccctctgagggtcggggtgttggtgatattgatgcatcaactgattacaacatggaagatgccttattgaacgacgaaactcgacagcctctatctaggaaagttccacttccttcctccaggataaatccatacaggatggtcattgtgctgcgattgattgttctaagcatcttcttgcactaccgtatcacaaatcctgtgcgcaatgcatacccattatggcttctatctgttatatgtgagatctggtttgctctttcgtggatattggatcagttccctaagtggtttccaatcaaccgggagacgtaccttgataggctggcattaaggtatgaccgggaaggtgagccatctcagttggctgctgttgacattttcgtcagtacagtcgacccaatgaaggagcctcctcttgtcactgccaataccgtgctatccattcttgctgtggattaccctgtggataaggtctcttgctatgtatctgatgatggagctgcgatgctgacatttgatgcactagctgagacttcagagtttgctagaaaatgggtaccatttgttaagaagtacaacattgaacctagagctcctgaatggtacttctcccagaaaattgattacttgaaggacaaagtgcacccttcatttgttaaagaccgccgggccatgaagagagaatatgaagaattcaaagttagggtaaatggccttgttgctaaggcacagaaagttcctgaggaaggatggatcatgcaagatggcacaccatggccaggaaacaataccagggaccatcctggaatgattcaggttttccttggtcacagtggtggccttgatactgagggcaatgagctaccccgtttggtctatgtttctcgtgaaaagcgtcctggattccagcatcacaagaaagctggtgccatgaatgctcttgttcgtgtctcagctgtgcttaccaatggacaatacatgttgaatcttgattgtgatcactacattaacaacagtaaggctctcagggaagctatgtgcttccttatggaccctaacctaggaaggagtgtctgctacgtccagtttccccagagattcgatggcattgacaggaatgatcgatatgccaacaggaacaccgtgtttttcgatattaacttgagaggtcttgatggcatccaaggaccagtttatgtcggaactggctgtgttttcaaccgaacagctctatatggttatgagcccccaattaagcagaagaagggtggtttcttgtcatcactatgtggcggtaggaagaaggcaagcaaatcaaagaagggctcggacaagaagaagtcgcagaagcatgtggacagttctgtgccagtattcaaccttgaagatatagaggagggagttgaaggcgctggatttgacgacgagaaatcacttcttatgtctcaaatgagcctggagaagagatttggccagtccgcagcgtttgttgcctccactctgatggagtatggtggtgttcctcagtccgcaactccggagtctcttctgaaagaagctatccatgttataagctgtggctatgaggacaagactgaatggggaactgagatcgggtggatctacggttctgtgacagaagacattctcaccggattcaagatgcacgcgcgaggctggcggtcgatctactgcatgcccaagcggccagctttcaaggggtctgcccccatcaatctttcggaccgtctgaaccaggtgctccggtgggctcttgggtccgtggagatcctcttcagccggcactgccccctgtggtacggctacggagggcggctcaagttcctggagagattcgcgtacatcaacaccaccatctacccgctcacgtccatcccgcttctcatctactgcatcctgcccgccatctgtctgctcaccggaaagttcatcattccagagatcagcaacttcgccagcatctggttcatctccctcttcatctcgatcttcgccacgggcatcctggagatgaggtggagcggggtgggcatcgacgagtggtggaggaacgagcagttctgggtgatcgggggcatctccgcgcacctcttcgccgtgttccagggcctgctcaaggtgctggccggcatcgacaccaacttcaccgtcacctccaaggcctcggacgaggacggcgacttcgcggagctgtacatgttcaagtggacgacgctcctgatcccgcccaccaccatcctgatcatcaacctggtcggcgtcgtcgccggcatctcctacgccatcaacagcggataccagtcgtggggcccgctcttcggcaagctcttcttcgccttctgggtcatcgtccacctgtacccgttcctcaagggcctcatgggcaggcagaaccgcaccccgaccatcgtcgtcgtctgggccatcctgctggcgtccatcttctccttgctgtgggttcgcatcgaccccttcaccacccgcgtcactggcccggatacccagacgtgtggcatcaactgctagggaagtggaaggtttgtactttgtagaaacggaggaataccacgtgccatctgttgtctgttaagttatatatatataagcagcaagtggcgttatttacagctacgtacagaccagtggatattgtttaccacaaagttttacttgtgttaatatgcattcttttgttgatataaaaaaaaaaaaaaaaaagggcggccgc

SEQ ID NO: 10 - protein, 1077 aa, Zea mays
MEGDADGVKSGRRGGGQVCQICGDGVGTTAEGDVFAACDVCGFPVCRPCYEYERKDGTQACPQCKTKYKRHKGSPAIRGEEGDDTDADSDFNYLASGNEDQKQKIADRMRSWRMNVGGSGDVGRPKYDSGEIGLTKYDSGEIPRGYIPSVTNSQISGEIPGASPDHHMMSPTGNIGKRAPFPYVNHSPNPSREFSGSIGNVAWKERVDGWKMKQDKGTIPMTNGTSIAPSEGRGVGDIDASTDYNMEDALLNDETRQPLSRKVPLPSSRINPYRMVIVLRLIVLSIFLHYRITNPVRNAYPLWLLSVICEIWFALSWILDQFPKWFPINRETYLDRLALRYDREGEPSQLAAVDIFVSTVDPMKEPPLVTANTVLSILAVDYPVDKVSCYVSDDGAAMLTFDALAETSEFARKWVPFVKKYNIEPRAPEWYFSQKIDYLKDKVHPSFVKDRRAMKREYEEFKVRVNGLVAKAQKVPEEGWIMQDGTPWPGNNTRDHPGMIQVFLGHSGGLDTEGNELPRLVYVSREKRPGFQHHKKAGAMNALVRVSAVLTNGQYMLNLDCDHYINNSKALREAMCFLMDPNLGRSVCYVQFPQRFDGIDRNDRYANRNTVFFDINLRGLDGIQGPVYVGTGCVFNRTALYGYEPPIKQKKGGFLSSLCGGRKKASKSKKGSDKKKSQKHVDSSVPVFNLEDIEEGVEGAGFDDEKSLLMSQMSLEKRFGQSAAFVASTLMEYGGVPQSATPESLLKEAIHVISCGYEDKTEWGTEIGWIYGSVTEDILTGFKMHARGWRSIYCMPKRPAFKGSAPINLSDRLNQVLRWALGSVEILFSRHCPLWYGYGGRLKFLERFAYINTTIYPLTSIPLLIYCILPAICLLTGKFIIPEISNFASIWFISLFISIFATGILEMRWSGVGIDEWWRNEQFWVIGGISAHLFAVFQGLLKVLAGIDTNFTVTSKASDEDGDFAELYMFKWTTLLIPPTTILIINLVGVVAGISYAINSGYQSWGPLFGKLFFAFWVIVHLYPFLKGLMGRQNRTPTIVVVWAILLASIFSLLWVRIDPFTTRVTGPDTQTCGINC

SEQ ID NO: 11 - DNA, 25 bp, Zea mays
atggagggcgacgcggacggcgtga

SEQ ID NO: 12 - DNA, 25 bp, Zea mays
ctagcagttgatgccacacgtctgg

SEQ ID NO: 13 - DNA, 3704 bp, Zea mays
gtcgacccacgcttccggtcggttccgcgtcccttttcccctcccccctccgtcgccgcctcgagcgagctccaccacttgctcctgcgcgaggtgaacactgggttagggccactgccaccgctgggctgcctctgcttctgcctctcccgccagcgcgcgagcccgggggcgattcggcgccggcacgcgggaggggaagccgaggaatgcggtgagtcggcgggggtccggcgtttgtgaactcgtggagggctcggattggtgcgccatggacggcggcgacgccacgaattcggggaagcatgtggccgggcaggtgtgccagatctgcggcgacggcgtgggcaccgcggcggacggcgacctcttcaccgcctgcgacgtctgcggcttccccgtgtgccgcccatgctacgagtacgagcgcaaggacggcacccaggcgtgcccgcagtgcaagactaagtacaagcgccacaaagggagcccaccagtacacggtgaggaaaatgaggatgtggatgctgacgatgtgagtgactacaactaccaagcatctggcaaccaggatcagaagcaaaagattgctgagagaatgctcacttggcggacaaactcacgtggcagtgatattggcctggctaagtatgacagcggtgaaattgggcatgggaagtatgacagtggtgagatccctcgtggatatatcccgtcactaactcatagccagatctcaggagagattcctggagcttcccctgatcatatgatgtctcctgttgggaacattggcaggcgtggacatcaatttccttatgtaaatcattctccaaacccatcgagggagttctccggtagccttggcaatgttgcatggaaagagagggtggatggatggaaaatgaaggataaaggtgcaattcctatgaccaatggaacaagcattgctccatcagaagggcgtggagttgctgatattgatgcttctactgattataacatggaagatgccttactgaatgatgaaactcggcaacctctatctagaaaagtgccaattccttcatccagaataaatccgtacagaatggtcattgtgctacgtttggctgttctatgcatattcttgcgctaccgtatcacacatcctgtgaacaatgcatatccactgtggcttttatccgtcatatgtgagatctggtttgctttgtcctggattttggatcagttcccaaagtggtccccaatcaaccgtgaaacataccttgatagactggctttaaggtatgaccgagaaggtgaaccatctcaattagctcctgttgatatttttgtcagtactgtggatccaatgaaggagcctcctcttgtcactgcaaatactgtgctttccatccttgctgtcgattatccggttgacaaggtatcttgctatgtttcggatgatggagctgctatgctgacttttgatgctctctctgaaacttcagagtttgctagaaaatgggttccgttctgtaagaagtacaacatagagcctagggccccggaatggtactttgctcagaaaattgattacttgaaagacaaagttcaaacctcatttgtgaaagaacgccgggccatgaagagagaatatgaagaattcaaagttcgtatcaatggtcttgtagccaaggcacaaaaagttcccgaggagggatggatcatgcaagatggtacaccttggcctgggaacaatactagggaccatcctggaatgattcaggttttcctgggtcacagtggagggcttgacgttgaaggcaatgaacttcctcgtttggtttatgtgtctcgtgaaaaacgtcctggattccaacatcacaagaaggctggtgccatgaatgcacttgttcgtgtatcagctgtccttactaatgggcaatacatgttgaatcttgattgtgaccactacatcaataatagcaaggctcttcgagaagctatgtgcttccttatggacccaaacctaggaaggaatgtctgttatgtccaatttcctcagaggtttgatggtattgataggaatgaccgatatgcaaacaggaacactgtgtttttcgatattaacttgagaggtcttgacggcattcaagggccagtttatgtgggaactggttgtgtgtttaacagaacggccttatatggttatgagcctccagtcaagaaaaaaaagccaggcttcttctcttcgctttgtgggggaaggaaaaagacgtcaaaatctaagaagagctcggaaaagaagaagtcacatagacacgcagacagttctgtaccagtatttaatctcgaagatatagaggaagggattgaaggttctcagtttgatgatgagaaatcgctgattatgtctcaaatgagcttggagaagagatttggccagtccagtgtttttgtagcctctactctgatggaatatggtggtgttccacaatctgcaactccagagtctcttctgaaagaagctattcatgtcatcagctgtggctatgaggacaaaactgactggggaactgagattgggtggatctatggttctgttacagaagacattctcaccggattcaagatgcatgctcgaggctggcgatcaatctactgcatgcctaagcgaccagctttcaagggatctgctcctatcaacctttcggatcgtttgaatcaagtgcttcggtgggctcttggttccattgaaattcttttcagcaggcattgtcccatatggtatggctatggaggccggcttaaattcctggagagatttgcttatatcaacacaacaatttatccactcacatcaatcccgctcctcctgtactgcatattgccagcagtttgtcttctcactgggaagttcatcatcccaaagattagtaacctagagagtgtttggtttatatcgctctttatctcaatctttgccactggtatccttgagatgaggtggagtggtgttggcattgatgaatggtggaggaacgagcagttctgggtcattggtggtatttctgcgcatttatttgccgtcttccagggtctcctgaaggtgcttgctggtatcgacacgagcttcactgtcacctctaaggccactgacgaagaaggtgattttgccgagctctacatgttcaagtggacaacgcttctgatcccaccaaccactattttgatcatcaacctggtcggcgtggtcgctggcatttcctacgcaatcaatagcggttaccagtcatggggacctcttttcgggaagctcttctttgcgttctgggtgattgtccacctgtaccccttcctcaagggcctcatggggaagcagaaccgcacgccgaccattgtcgttgtctgggctatcctccttgcgtcgatcttttccctgatgtgggttcgtatcgatccattcaccacccgggtcactggccctgatatcgcgaaatgtggcatcaactgctaggatgagctgaagatagttaaagagtggaactagacgcattgtgcatcgtaagttatcagtgggtggctctttttatagtatggtaggaacttggtcgggagacgttaattacatatgctatatgtacctccgctggtctttatccgtaagttaatatatatactgctttgagaattaaaaaaaaaaaaaaaagggcggccgc

SEQ ID NO: 14 - protein, 1076 aa, Zea mays
MDGGDATNSGKHVAGQVCQICGDGVGTAADGDLFTACDVCGFPVCRPCYEYERKDGTQACPQCKTKYKRHKGSPPVHGEENEDVDADDVSDYNYQASGNQDQKQKIAERMLTWRTNSRGSDIGLAKYDSGEIGHGKYDSGEIPRGYIPSLTHSQISGEIPGASPDHMMSPVGNIGRRGHQFPYVNHSPNPSREFSGSLGNVAWKERVDGWKMKDKGAIPMTNGTSIAPSEGRGVADIDASTDYNMEDALLNDETRQPLSRKVPIPSSRINPYRMVIVLRLAVLCIFLRYRITHPVNNAYPLWLLSVICEIWFALSWILDQFPKWSPINRETYLDRLALRYDREGEPSQLAPVDIFVSTVDPMKEPPLVTANTVLSILAVDYPVDKVSCYVSDDGAAMLTFDALSETSEFARKWVPFCKKYNIEPRAPEWYFAQKIDYLKDKVQTSFVKERRAMKREYEEFKVRINGLVAKAQKVPEEGWIMQDGTPWPGNNTRDHPGMIQVFLGHSGGLDVEGNELPRLVYVSREKRPGFQHHKKAGAMNALVRVSAVLTNGQYMLNLDCDHYINNSKALREAMCFLMDPNLGRNVCYVQFPQRFDGIDRNDRYANRNTVFFDINLRGLDGIQGPVYVGTGCVFNRTALYGYEPPVKKKKPGFFSSLCGGRKKTSKSKKSSEKKKSHRHADSSVPVFNLEDIEEGIEGSQFDDEKSLIMSQMSLEKRFGQSSVFVASTLMEYGGVPQSATPESLLKEAIHVISCGYEDKTDWGTEIGWIYGSVTEDILTGFKMHARGWRSIYCMPKRPAFKGSAPINLSDRLNQVLRWALGSIEILFSRHCPIWYGYGGRLKFLERFAYINTTIYPLTSIPLLLYCILPAVCLLTGKFIIPKISNLESVWFISLFISIFATGILEMRWSGVGIDEWWRNEQFWVIGGISAHLFAVFQGLLKVLAGIDTSFTVTSKATDEEGDFAELYMFKWTTLLIPPTTILIINLVGVVAGISYAINSGYQSWGPLFGKLFFAFWVIVHLYPFLKGLMGKQNRTPTIVVVWAILLASIFSLMWVRIDPFTTRVTGPDIAKCGINC

SEQ ID NO: 15 - DNA, 25 bp, Zea mays
atggacggcggcgacgccacgaatt

SEQ ID NO: 16 - DNA, 25 bp, Zea mays
ctagcagttgatgccacatttcgcg

SEQ ID NO: 17 - DNA, 3813 bp, Zea mays
ccacagctcatataccaagagccggagcagcttagcgcagcccagagcggcgccgcgccaagcacaacccccacccgccacagccgcgtgcgcatgtgagcggtcgccgcggccgggagaccagaggaggggaggactacgtgcatttcgctgtgccgccgccgcggggttcgtgcgcgagcgagatccggcggggcggggcggggggcctgagatggaggctagcgcggggctggtggccggctcgcataaccggaacgagctggtggtgatccgccgcgaccgcgagtcgggagccgcgggcggcggcgcggcgcgccgggcggaggcgccgtgccagatatgcggcgacgaggtcggggtgggcttcgacggggagcccttcgtggcgtgcaacgagtgcgccttccccgtctgccgcgcctgctacgagtacgagcgccgcgagggctcgcaagcgtgcccgcagtgcaggacccgctacaagcgcctcaagggctgcccgcgggtggccggcgacgaggaggaggacggcgtcgacgacctggagggcgagttcggcctgcaggacggcgccgcccacgaggacgacccgcagtacgtcgccgagtccatgctcagggcgcagatgagctacggccgcggcggcgacgcgcaccccggcttcagccccgtccccaacgtgccgctcctcaccaacggccagatggttgatgacatcccgccggagcagcacgcgctcgtgccgtcctacatgagcggcggcggcggcgggggcaagaggatccacccgctccctttcgcagatcccaaccttccagtgcaaccgagatccatggacccgtccaaggatctggccgcctacggatatggcagcgtggcctggaaggagagaatggagggctggaagcagaagcaggagcgcctgcagcatgtcaggagcgagggtggcggtgattgggatggcgacgatgcagatctgccactaatggatgaagctaggcagccattgtccagaaaagtccctatatcatcaagccgaattaatccctacaggatgattatcgttatccggttggtggttttgggtttcttcttccactaccgagtgatgcatccggcgaaagatgcatttgcattgtggctcatatctgtaatctgtgaaatctggtttgcgatgtcctggattcttgatcagttcccaaagtggcttccaatcgagagagagacttacctggaccgtttgtcactaaggtttgacaaggaaggtcaaccctctcagcttgctccaatcgacttctttgtcagtacggttgatcccacaaaggaacctcccttggtcacagcgaacactgtcctttccatcctttctgtggattatccggttgagaaggtctcctgctatgtttctgatgatggtgctgcaatgcttacgtttgaagcattgtctgaaacatctgaatttgcaaagaaatgggttcctttcagcaaaaagtttaatatcgagcctcgtgctcctgagtggtacttccaacagaagatagactacctgaaagacaaggttgctgcttcatttgttagggagaggagggcgatgaagagagaatacgaggaattcaaggtaaggatcaatgccttggttgcaaaagcccaaaaggttcctgaggaaggatggacaatgcaagatggaagcccctggcctggaaacaacgtacgcgatcatcctggaatgattcaggtattccttggccaaagtggcggtcgtgatgtggaaggaaatgagttgcctcgcctggtttatgtctcgagagaaaagaggccaggttataaccatcacaagaaggctggtgccatgaatgcactggtccgtgtctctgctgtcttatcaaatgctgcatacctattgaacttggactgtgatcactacatcaacaatagcaaggccataaaagaggctatgtgtttcatgatggatcctttggtggggaagaaagtgtgctatgtacagttccctcagaggtttgatggtattgacaaaaatgatcgatacgctaacaggaacgttgtcttttttgacatcaacatgaaaggtttggacggtattcaaggacccatttatgtgggtactggatgtgttttcagacggcaggcactgtatggttatgatgctcctaaaacgaagaagccaccatcaagaacttgcaactgctggcccaagtggtgcctctcttgctgctgcagcaggaacaagaataaaaagaagactacaaaaccaaagacggagaagaagaaaagattatttttcaagaaagcagaaaacccatctcctgcatatgctttgggtgaaattgatgaaggtgctccaggtgctgatatcgagaaggccggaatcgtaaatcaacagaaactagagaagaaatttgggcagtcttctgtttttgtcgcatcaacacttcttgagaacggagggaccctgaagagcgcaagtccagcttctcttctgaaggaagctatacatgttatcagctgcggctacgaagacaagaccgactggggaaaagagattggctggatttacggatcgatcacagaggatatcttgactggatttaagatgcactgccatggctggcggtctatttactgcatcccgaagcggcctgcattcaaaggttctgcgcctctgaacctttccgaccgtcttcaccaggtccttcgctgggcccttgggtccgtcgaaattttcttcagcaagcactgcccactttggtacggatacggcggcgggctaaaattcctggaaaggttttcttatatcaactccatcgtttatccctggacgtccattcctctcctggcttactgtaccttgcctgccatctgcctgctcacggggaagtttatcacaccagagcttaccaatgtcgccagtatctggttcatggcacttttcatctgcatctccgtgaccggcatcctggaaatgaggtggagtggcgtggccatcgacgactggtggaggaacgagcagttctgggtcatcggaggcgtttcggcgcatctgttcgcggtgttccagggcctgctgaaggtgttcgccggcatcgacacgagcttcaccgtgacgtcgaaggccggggacgacgaggagttctcggagctgtacacgttcaagtggaccaccctgctgatacccccgaccacgctcctcctgctgaacttcatcggggtggtggccgggatctcgaacgcgatcaacaacgggtacgagtcgtggggccccctgttcgggaagctcttcttcgccttctgggtgatcgtccacctgtacccgttcctcaagggtctggtggggaggcagaacaggacgccgacgatcgtcatcgtctggtccatcctgctggcctcgatcttctcgctcctgtgggtccgcgtcgacccgttcctcgccaagagcaacggcccgctcctggaggagtgtggcctggactgcaactgaagtgggggccccctgtcactcgaagttctgtcacgggcgaattacgcctgattttttgttgttgttgttgttggaattctttgctgtagatagaaaccacatgtccacggcatctctgctgtgtccattggagcaggagagaggtgcctgctgctgtttgttgagtaaattaaaagttttaaagttatacagtgatgcacattccagtgcccagtgtattccctttttacagtctgtatattagcgacaaaggacatattggttaggagtttgattcttttgtaaaaaaaaaaaaaaaaaaaaaaaaaaaaaaa

SEQ ID NO: 18 - protein, 1094 aa, Zea mays
MEASAGLVAGSHNRNELVVIRRDRESGAAGGGAARRAEAPCQICGDEVGVGFDGEPFVACNECAFPVCRACYEYERREGSQACPQCRTRYKRLKGCPRVAGDEEEDGVDDLEGEFGLQDGAAHEDDPQYVAESMLRAQMSYGRGGDAHPGFSPVPNVPLLTNGQMVDDIPPEQHALVPSYMSGGGGGGKRIHPLPFADPNLPVQPRSMDPSKDLAAYGYGSVAWKERMEGWKQKQERLQHVRSEGGGDWDGDDADLPLMDEARQPLSRKVPISSSRINPYRMIIVIRLVVLGFFFHYRVMHPAKDAFALWLISVICEIWFAMSWILDQFPKWLPIERETYLDRLSLRFDKEGQPSQLAPIDFFVSTVDPTKEPPLVTANTVLSILSVDYPVEKVSCYVSDDGAAMLTFEALSETSEFAKKWVPFSKKFNIEPRAPEWYFQQKIDYLKDKVAASFVRERRAMKREYEEFKVRINALVAKAQKVPEEGWTMQDGSPWPGNNVRDHPGMIQVFLGQSGGRDVEGNELPRLVYVSREKRPGYNHHKKAGAMNALVRVSAVLSNAAYLLNLDCDHYINNSKAIKEAMCFMMDPLVGKKVCYVQFPQRFDGIDKNDRYANRNVVFFDINMKGLDGIQGPIYVGTGCVFRRQALYGYDAPKTKKPPSRTCNCWPKWCLSCCCSRNKNKKKTTKPKTEKKKRLFFKKAENPSPAYALGEIDEGAPGADIEKAGIVNQQKLEKKFGQSSVFVASTLLENGGTLKSASPASLLKEAIHVISCGYEDKTDWGKEIGWIYGSITEDILTGFKMHCHGWRSIYCIPKRPAFKGSAPLNLSDRLHQVLRWALGSVEIFFSKHCPLWYGYGGGLKFLERFSYINSIVYPWTSIPLLAYCTLPAICLLTGKFITPELTNVASIWFMALFICISVTGILEMRWSGVAIDDWWRNEQFWVIGGVSAHLFAVFQGLLKVFAGIDTSFTVTSKAGDDEEFSELYTFKWTTLLIPPTTLLLLNFIGVVAGISNAINNGYESWGPLFGKLFFAFWVIVHLYPFLKGLVGRQNRTPTIVIVWSILLASIFSLLWVRVDPFLAKSNGPLLEECGLDCN

SEQ ID NO: 19 - DNA, 25 bp, Zea mays
atggaggctagcgcggggctggtgg

SEQ ID NO: 20 - DNA, 25 bp, Zea mays
tcagttgcagtccaggccacactcc

SEQ ID NO: 21 - DNA, 3799 bp, Zea mays
  misc_feature 3757, 3775, 3777, 3782: n = A,T,C or G
caactcacgttgccgcggcttcctccatcggtgcggtgccctgtccttttctctcctccacctccctagtccctcctcccccccgcatacatagctactactagtagcaccacgctcgcagcgggagatgcggtgctgatccgtgcccctgctcggatctcgggagtggtgccgacttgtgtcgcttcggctctgcctaggccagctccttgtcggttctgggcgagctcgcctgccatggagggcgacgcggacggcgtgaagtcggggaggcgcgggggagggcaggtgtgccagatctgcggcgatggcgtgggcactacggcggagggagacgtcttcaccgcctgcgacgtctgcgggttcccggtgtgccgcccctgctacgagtacgagcgcaaggacggcacacaagcgtgcccccagtgcaaaaacaagtacaagcgccacaaggggagtccagcgatccgaggggaggaaggagacgatactgatgccgatgatgctagcgacttcaactaccctgcatctggcaatgacgaccagaagcagaagattgctgacaggatgcgcagctggcgcatgaatgctgggggcagcggggatgttggccgccccaagtatgacagtggtgagatcgggcttaccaagtacgacagtggtgagatccctcggggatacatcccgtcagtcactaacagccagatttcgggagaaatccctggtgcttcccctgaccatcatatgatgtctcctactgggaacattggcaggcgcgccccatttccctatatgaatcattcatcaaatccgtcgagggaattctctggtagcgttgggaatgttgcctggaaagagagggttgatggctggaaaatgaagcaggacaagggaacaattcccatgacgaatggcacaagcattgctccctctgagggccggggtgttggtgatattgatgcatcaactgattacaacatggaagatgccttattaaacgatgaaactcgccagcctctatctaggaaagttccacttccttcctccaggataaatccatacaggatggtcattgtgctacgattgattgttctaagcatcttcttgcactaccggatcacaaatcctgtgcgtaatgcatacccactgtggcttctatctgttatatgtgagatctggtttgctctttcctggatattggatcagtttccaaagtggtttccaatcaaccgcgagacttaccttgatagactcgcattaaggtatgaccgggaaggtgagccatctcagttggctgctgttgacatttttgtcagtactgtcgacccaatgaaggagcctcctcttgtcactgccaataccgtgctatccattctcgctgtggactatcctgtggataaggtctcttgctatgtatctgatgatggagctgctatgctgacatttgatgcactagctgagacttcagagtttgctagaaaatgggtgccatttgttaagaagtacaacattgaacctagagctcctgaatggtacttctcccagaaaattgattacttgaaggacaaagtgcacccttcatttgttaaagaccgccgggccatgaagagagaatatgaagaattcaaaattagggtaaatggccttgttgctaaggcacaaaaagtccctgaggaaggatggatcatgcaagatggcacaccatggccaggaaacaataccagggaccatcctggaatgattcaggttttccttggtcacagtggtggtcttgatactgagggtaatgagctaccccgtttggtctatgtttctcgtgaaaaacgtcctggattccagcatcacaagaaagctggtgccatgaatgctcttgtccgcgtctcagctgtgcttaccaatggacaatacatgttgaatcttgattgtgatcactacatcaacaacagtaaggctctcagggaagctatgtgcttccttatggatcctaacctaggaaggagtgtctgctatgttcagtttccccagaggttcgatggtattgataggaatgatcgatatgccaacaggaacaccgtgtttttcgatattaacttgagaggtcttgatggcatccaaggaccagtttatgtgggcactggctgtgttttcaacagaacagctctatatggttatgagcccccaattaagcaaaagaagggtggtttcttgtcatcactatgtggtggcaggaagaagggaagcaaatcaaagaagggctcagacaagaaaaagtcacagaagcatgtggacagttctgtgccagtattcaatcttgaagatatagaggagggagttgaaggcgctggatttgatgatgagaaatcacttcttatgtctcaaatgagcttggagaagagatttggccaatctgcagcttttgttgcgtccactctgatggaatatggtggtgttcctcagtctgcgactccagaatctcttctgaaagaagctatccatgtcataagttgtggctacgaggacaagattgaatggggaactgagattgggtggatctatggttctgtgacggaagatattctcactgggttcaagatgcacgcacgaggctggcggtcgatctactgcatgcctaagcggccggccttcaagggatcggctcccatcaatctctcagaccgtctgaaccaggtgctccggtgggctctcggttcagtggaaatccttttcagccggcattgccccctatggtacgggtacggaggacgcctgaagttcttggagagattcgcctacatcaacaccaccatctacccgctcacgtccctcccgctcctcatttactgtatcctgcctgccatctgcctgctcacggggaagttcatcatcccagagatcagcaacttcgctagtatctggttcatctctctcttcatctcgatcttcgccacgggtatcctggagatgaggtggagcggcgtgggcatcgacgagtggtggaggaacgagcagttctgggtcatcggaggcatctccgcccacctcttcgccgtcttccagggcctcctcaaggtgcttgccggcatcgacaccaacttcaccgtcacctccaaggcctcggatgaagacggcgacttcgcggagctgtacatgttcaagtggacgacacttctgatcccgcccaccaccatcctgatcatcaacctggtcggcgttgttgccggcatctcctacgccatcaacagcgggtaccagtcgtggggtccgctcttcggcaagctcttcttcgccttctgggtgatcgttcacctgtacccgttcctcaagggtctcatgggtcggcagaaccgcaccccgaccatcgtggttgtctgggcgatcctgctggcgtcgatcttctccttgctgtgggttcgcatcgatccgttcaccaaccgcgtcactggcccggatactcgaacgtgtggcatcaactgctagggaggtggaaggtttgtagaaacagagagataccacgaatgtgccgctgccacaaattgtctgttagtaagttatataggcaggtggcgttatttacagctacgtacacacaaggggatactccgtttatcactggtgtgcattcttttgttgatataagttactatatatacgtattgcttctactttgtggagagtggctgacaggaccagttttgtaatgttatgaacagcaaagaaataagttagtttccaaaaaaaaaaaaaaaaaaaaaaaaanaaaaaaaaaaaaaaaaananaaaanaaaaaaaaaaaaacccc

SEQ ID NO: 22 - protein, 1079 aa, Zea mays
MEGDADGVKSGRRGGGQVCQICGDGVGTTAEGDVFTACDVCGFPVCRPCYEYERKDGTQACPQCKNKYKRHKGSPAIRGEEGDDTDADDASDFNYPASGNDDQKQKIADRMRSWRMNAGGSGDVGRPKYDSGEIGLTKYDSGEIPRGYIPSVTNSQISGEIPGASPDHHMMSPTGNIGRRAPFPYMNHSSNPSREFSGSVGNVAWKERVDGWKMKQDKGTIPMTNGTSIAPSEGRGVGDIDASTDYNMEDALLNDETRQPLSRKVPLPSSRINPYRMVIVLRLIVLSIFLHYRITNPVRNAYPLWLLSVICEIWFALSWILDQFPKWFPINRETYLDRLALRYDREGEPSQLAAVDIFVSTVDPMKEPPLVTANTVLSILAVDYPVDKVSCYVSDDGAAMLTFDALAETSEFARKWVPFVKKYNIEPRAPEWYFSQKIDYLKDKVHPSFVKDRRAMKREYEEFKIRVNGLVAKAQKVPEEGWIMQDGTPWPGNNTRDHPGMIQVFLGHSGGLDTEGNELPRLVYVSREKRPGFQHHKKAGAMNALVRVSAVLTNGQYMLNLDCDHYINNSKALREAMCFLMDPNLGRSVCYVQFPQRFDGIDRNDRYANRNTVFFDINLRGLDGIQGPVYVGTGCVFNRTALYGYEPPIKQKKGGFLSSLCGGRKKGSKSKKGSDKKKSQKHVDSSVPVFNLEDIEEGVEGAGFDDEKSLLMSQMSLEKRFGQSAAFVASTLMEYGGVPQSATPESLLKEAIHVISCGYEDKIEWGTEIGWIYGSVTEDILTGFKMHARGWRSIYCMPKRPAFKGSAPINLSDRLNQVLRWALGSVEILFSRHCPLWYGYGGRLKFLERFAYINTTIYPLTSLPLLIYCILPAICLLTGKFIIPEISNFASIWFISLFISIFATGILEMRWSGVGIDEWWRNEQFWVIGGISAHLFAVFQGLLKVLAGIDTNFTVTSKASDEDGDFAELYMFKWTTLLIPPTTILIINLVGVVAGISYAINSGYQSWGPLFGKLFFAFWVIVHLYPFLKGLMGRQNRTPTIVVVWAILLASIFSLLWVRIDPFTNRVTGPDTRTCGINC

SEQ ID NO: 23 - DNA, 25 bp, Zea mays
atggagggcgacgcggacggcgtga

SEQ ID NO: 24 - DNA, 25 bp, Zea mays
ctagcagttgatgccacacgttcga

SEQ ID NO: 25 - DNA, 3470 bp, Zea mays
gcccccggtcgatcgctcggcaatcggcatggacgccggctcggtcaccggtggcctcgccgcgggctcgcacatgcgggacgagctgcatgtcatgcgcgcccgcgaggagccgaacgccaaggtccggagcgccgacgtgaagacgtgccgcgtgtgcgccgacgaggtcgggacgcgggaggacgggcagcccttcgtggcgtgcgccgagtgcggcttccccgtctgccggccctgctacgagtacgagcgcagcgagggcacgcagtgctgcccgcagtgcaacacccgctacaagcgccagaaagggtgcccgagggtggaaggggacgaggaggagggcccggagatggacgacttcgaggacgagttccccgccaagagccccaagaagcctcacgagcctgtcgcgttcgacgtctactcggagaacggcgagcacccggcgcagaaatggcggacgggtggccagacgctgtcgtccttcaccggaagcgtcgccgggaaggacctggaggcggagagggagatggaggggagcatggagtggaaggaccggatcgacaagtggaagaccaagcaggagaagaggggcaagctcaaccacgacgacagcgacgacgacgacgacaagaacgaagacgagtacatgctgcttgccgaggcccgacagccgctgtggcgcaaggttccgatcccgtcgagcatgatcaacccgtaccgcatcgtcatcgtgctccgcctggtggtgctctgcttcttcctcaagttccggatcacgacgcccgccacggacgccgtgcctctgtggctggcgtccgtcatctgcgagctctggttcgccttctcctggatcctggaccagctgccaaagtgggcgccggtgacgcgggagacgtacctggaccgcctggcgctgcggtacgaccgtgagggcgaggcgtgccggctgtcccccatcgacttcttcgtcagcacggtggacccgctcaaggagccgcccatcatcaccgccaacaccgtgctgtccatcctcgccgtcgactaccccgtggaccgcgtcagctgctacgtctccgacgacggcgcgtccatgctgctcttcgacgcgctgtccgagaccgccgagttcgcgcgccgctgggtgcccttctgcaagaagttcgccgtggagccgcgcgccccggagttctacttctcgcagaagatcgactacctcaaggacaaggtgcagccgacgttcgtcaaggagcgccgcgccatgaagagggagtacgaggagttcaaggtgcgcatcaacgcgctggtggccaaggcgcagaagaagcccgaggaggggtgggtcatgcaggacggcacgccgtggcccgggaacaacacgcgcgaccacccgggtatgatccaggtctacctcggcaaccagggcgcgctggacgtggagggccacgagctgccgcgcctcgtctacgtgtcccgtgagaagcgccccgggtacaaccaccacaagaaggcgggcgccatgaacgcgctggtgcgcgtctccgccgtgctcaccaacgcgcccttcatcctcaacctcgactgcgaccactacgtcaacaacagcaaggccgtgcgcgaggccatgtgcttcctcatggacccgcagctggggaagaagctctgctacgtccagttcccgcagcgcttcgatggcatcgatcgccacgaccgatacgccaaccgcaacgtcgtcttcttcgacatcaacatgaaggggctggacggcatccagggcccggtgtacgtcggcacggggtgcgtgttcaaccgccaggcgctgtacggctacgacccgccgcggcccgagaagcggcccaagatgacgtgcgactgctggccgtcgtggtgctgctgctgctgctgcttcggcggcggcaagcgcggcaaggcgcgcaaggacaagaagggcgacggcggcgaggagccgcgccggggcctgctcggcttctacaggaagcggagcaagaaggacaagctcggcggcgggtcggtggccggcagcaagaagggcggcgggctgtacaagaagcaccagcgcgcgttcgagctggaggagatcgaggaggggctggaggggtacgacgagctggagcgctcctcgctcatgtcgcagaagagcttcgagaagcggttcggccagtcgcccgtgttcatcgcctccacgctcgtcgaggacggcggcctgccgcagggcgccgccgccgaccccgccgcgctcatcaaggaggccatccacgtcatcagctgcggatacgaggagaagaccgagtggggcaaggagattgggtggatctatgggtcggtgacagaggatatcctgacggggttcaagatgcactgccgggggtggaagtccgtgtactgcacgccgacacggccggcgttcaaggggtcggcgcccatcaacttgtctgatcgtctccaccaggtgctgcgctgggcgctggggtccgtggagatcttcatgagccgccactgcccgctccggtacgcctacggcggccggctcaagtggctggagcgcttcgcctacaccaacaccatcgtgtaccccttcacctccatcccgctcctcgcctactgcaccatccccgccgtctgcctgctcaccggcaagttcatcattcccacgctgaacaacctcgccagcatctggttcatcgcgctcttcctgtccatcatcgcgacgagcgtcctggagctgcggtggagcggggtgagcatcgaggactggtggcgcaacgagcagttctgggtcatcggcggcgtgtccgcgcatctcttcgccgtgttccagggcttcctcaaggttctgggcggcgtggacaccagcttcaccgtcacctccaaggcggccggcgacgaggccgacgccttcggggacctctacctcttcaagtggaccaccctgctggtgccccccaccacgctcatcatcatcaacatggtgggcatcgtggccggcgtgtccgacgccgtcaacaacggctacggctcctggggcccgctcttcggcaagctcttcttctccttctgggtcatcgtccacctctacccgttcctcaaggggctcatggggaggcagaaccggacgcccaccatcgtcgtgctctggtccatcctcctcgcctccatcttctcgctcgtctgggtcaggatcgacccgtttatcccgaaggccaagggccccatcctcaagccatgcggagtcgagtgctgagctcacctagctaccttcttgttgcatgtacggacgccgccgtgcgtttggacatacaggcacttttgggccaggctactcatgttcgactttttttttaattttgtacaagatttgtgatcgagtgactgagtgagacagagtgttgggtgtaagaactgtgatggaattcactcaaattaatggacattttttttcttcaaaa

SEQ ID NO: 26 - protein, 1078 aa, Zea mays
MDAGSVTGGLAAGSHMRDELHVMRAREEPNAKVRSADVKTCRVCADEVGTREDGQPFVACAECGFPVCRPCYEYERSEGTQCCPQCNTRYKRQKGCPRVEGDEEEGPEMDDFEDEFPAKSPKKPHEPVAFDVYSENGEHPAQKWRTGGQTLSSFTGSVAGKDLEAEREMEGSMEWKDRIDKWKTKQEKRGKLNHDDSDDDDDKNEDEYMLLAEARQPLWRKVPIPSSMINPYRIVIVLRLVVLCFFLKFRITTPATDAVPLWLASVICELWFAFSWILDQLPKWAPVTRETYLDRLALRYDREGEACRLSPIDFFVSTVDPLKEPPIITANTVLSILAVDYPVDRVSCYVSDDGASMLLFDALSETAEFARRWVPFCKKFAVEPRAPEFYFSQKIDYLKDKVQPTFVKERRAMKREYEEFKVRINALVAKAQKKPEEGWVMQDGTPWPGNNTRDHPGMIQVYLGNQGALDVEGHELPRLVYVSREKRPGYNHHKKAGAMNALVRVSAVLTNAPFILNLDCDHYVNNSKAVREAMCFLMDPQLGKKLCYVQFPQRFDGIDRHDRYANRNVVFFDINMKGLDGIQGPVYVGTGCVFNRQALYGYDPPRPEKRPKMTCDCWPSWCCCCCCFGGGKRGKARKDKKGDGGEEPRRGLLGFYRKRSKKDKLGGGSVAGSKKGGGLYKKHQRAFELEEIEEGLEGYDELERSSLMSQKSFEKRFGQSPVFIASTLVEDGGLPQGAAADPAALIKEAIHVISCGYEEKTEWGKEIGWIYGSVTEDILTGFKMHCRGWKSVYCTPTRPAFKGSAPINLSDRLHQVLRWALGSVEIFMSRHCPLRYAYGGRLKWLERFAYTNTIVYPFTSIPLLAYCTIPAVCLLTGKFIIPTLNNLASIWFIALFLSIIATSVLELRWSGVSIEDWWRNEQFWVIGGVSAHLFAVFQGFLKVLGGVDTSFTVTSKAAGDEADAFGDLYLFKWTTLLVPPTTLIIINMVGIVAGVSDAVNNGYGSWGPLFGKLFFSFWVIVHLYPFLKGLMGRQNRTPTIVVLWSILLASIFSLVWVRIDPFIPKAKGPILKPCGVEC

SEQ ID NO: 27 - DNA, 3231 bp, Zea mays
ccacgcgtccgggaggggccatgatggagtcggcggcggcccagtcctgcgcggcgtgcggggacgacgcgcgcgctgcctgccgcgcgtgcagctacgcgctctgcagggcgtgcctcgacgaggacgccgccgagggccgcaccacatgcgcgcgctgcggaggggactacgccgctatcaacccagcgcgcgccagcgagggaaccgaggcggaggaggaggtggtggagaaccaccacaccgccggtggcctgcgtgagagggtcaccatgggcagccacctcaatgatcgccaggatgaagtaagccacgccaggaccatgagcagcttgtcgggaattggtagtgaattgaatgatgaatctggtaagcccatctggaagaacagggtggagagttggaaggaaaagaagaatgagaagaaagcctcggccaaaaagactgcagctaaagcacagcctccgcctgtcgaagaacagatcatggatgaaaaagacttgacagatgcatatgagccactctcccgggtcatcccaatatcaaagaacaagctcacaccttacagagcagtgatcattatgcggttaattgttcttgggctcttctttcactaccgtatcaccaatcctgttaacagtgcctttggtctctggatgacatcagttatatgtgagatctggtttggtttctcctggatattggatcaattcccgaagtggtatcctatcaatcgtgagacttatgttgataggctgattgcacgatatggagatggtgaagaatctgggttagcacctgtagatttctttgtcagtacagtggatccattgaaagagcctccactaatcactgcaaacactgtgctgtctattcttgctgtggactatcccgttgagaagatctcatgctatgtatctgatgatggttctgctatgctcacatttgaatcgctcgcagagactgcagaatatgctagaaagtgggtgccgttttgcaagaagtacgccattgagccacgagctcctgagttctacttctcacagaaaattgactacttgaaggacaagatacacccatcttttgtcaaggagcgtagggctatgaagagagactatgaagagtacaaggtgaggataaatgctttggttgccaaggctcaaaagacacctgatgaaggctggatcatgcaagacggtacaccatggcctgggaacaatcctcgtgaccaccctggcatgatccaggttttcctgggtgagactggtgcacgggactttgatggaaatgaacttcctcggttagtgtatgtgtcaagagagaaaagaccaggctaccaacaccacaagaaggcaggggctatgaatgctctggtccgagtgtctgctgttctgacaaatgccccttacattcttaatcttgattgtgatcactatgttaacaacagcaaagctgttcgtgaagcaatgtgcttcatgatggaccctactgttggcagagatgtctgctatgtacaattcccccagaggttcgatggcattgatcgcagtgatcgatatgccaataggaacgttgtgttctttgatgttaatatgaaaggacttgatggcctccaaggcccagtttatgtgggaactggttgttgtttcaataggcaagcactttatggttatgggcctccatctctgcccgcacttccaaagtcttcgatttgttcctggtgttgctgctgctgtcccaagaaaaaggttgaaagaagtgagagggaaatcaacagagactctcggcgagaagacctcgagtctgccatttttaaccttcgcgaaattgacaactacgatgagtacgagaggtccatgctcatctctcagatgagcttcgagaagtcttttgggctgtcctcggtctttattgaatcgacccttatggagaatgggggcgtccctgaatctgcaaacccatctaccctaattaaagaagccattcatgtcattagctgtggatatgaagagaaaactgaatggggaaaagagattggctggatctatggttcagttacagaggatattctgactgggtttaagatgcactgccgtggctggagatccatctactgcatgccggtgagacctgcattcaagggatcagccccaatcaatctttccgatcgtcttcaccaagttctccggtgggctcttgtttctgtcgagatcttcttcagtcggcactgcccgctgtggtacggttacggtggcggccgtctgaaatggctccagaggctctcctacatcaacaccatcgtgtacccgttcacttctcttcctctcgttgcctactgttgcctgcctgccatttgcctgctcacaggaaagttcattatacctacgctgtccaacgctgcaacgatatggtttcttggcctcttcatgtccatcatcgtgacgagcgtgttggagctgcggtggagtggcatcgggatcgaggactggtggcgcaacgagcagttctgggtcatcggaggcgtgtccgcgcacctgttcgccgtgttccagggtatcctcaagatgattgccgggctggacaccaacttcacggtcacggcaaaggccacggacgacactgagttcggggagctgtacctgttcaagtggacgacggtgctgatcccgcccacaagcatcctggtgctgaacctggtgggcgtggtggctgggttctcggccgcgctcaacagcggctacgagtcctggggcccgctcttcggtaaggtgttcttcgccatgtgggtgatcatgcacctgtacccgttcctcaagggtctcatgggccgccagaaccgcacgccgaccatcgtggtgctctggtccgtcctcctcgcctccgtcttctccctcctgtgggtcaagatcgacccattcgttggaggaaccgagaccgtcaacaccaacaactgcaacacacatctgctgattcaccatcggtcagctgctgtcgtgccgcggcggacgtgtttctggtgttgcaaacgtgggttgcctgcctgatgcgggtctcctctgtctatctcgcatctgggcttttgccccaggatctgaagcgggtggtgtaggttagctttattttgcgtccaagtgttgattgatgttgtctgtgttatgaaaagttttggtggtgaaacctgaaatgttaaaattcggctcaattgtgagaaaaaaaaaaaaaaaaaaaaaa

SEQ ID NO: 28 - protein, 1007 aa, Zea mays
MMESAAAQSCAACGDDARAACRACSYALCRACLDEDAAEGRTTCARCGGDYAAINPARASEGTEAEEEVVENHHTAGGLRERVTMGSHLNDRQDEVSHARTMSSLSGIGSELNDESGKPIWKNRVESWKEKKNEKKASAKKTAAKAQPPPVEEQIMDEKDLTDAYEPLSRVIPISKNKLTPYRAVIIMRLIVLGLFFHYRITNPVNSAFGLWMTSVICEIWFGFSWILDQFPKWYPINRETYVDRLIARYGDGEESGLAPVDFFVSTVDPLKEPPLITANTVLSILAVDYPVEKISCYVSDDGSAMLTFESLAETAEYARKWVPFCKKYAIEPRAPEFYFSQKIDYLKDKIHPSFVKERRAMKRDYEEYKVRINALVAKAQKTPDEGWIMQDGTPWPGNNPRDHPGMIQVFLGETGARDFDGNELPRLVYVSREKRPGYQHHKKAGAMNALVRVSAVLTNAPYILNLDCDHYVNNSKAVREAMCFMMDPTVGRDVCYVQFPQRFDGIDRSDRYANRNVVFFDVNMKGLDGLQGPVYVGTGCCFNRQALYGYGPPSLPALPKSSICSWCCCCCPKKKVERSEREINRDSRREDLESAIFNLREIDNYDEYERSMLISQMSFEKSFGLSSVFIESTLMENGGVPESANPSTLIKEAIHVISCGYEEKTEWGKEIGWIYGSVTEDILTGFKMHCRGWRSIYCMPVRPAFKGSAPINLSDRLHQVLRWALVSVEIFFSRHCPLWYGYGGGRLKWLQRLSYINTIVYPFTSLPLVAYCCLPAICLLTGKFIIPTLSNAATIWFLGLFMSIIVTSVLELRWSGIGIEDWWRNEQFWVIGGVSAHLFAVFQGILKMIAGLDTNFTVTAKATDDTEFGELYLFKWTTVLIPPTSILVLNLVGVVAGFSAALNSGYESWGPLFGKVFFAMWVIMHLYPFLKGLMGRQNRTPTIVVLWSVLLASVFSLLWVKIDPFVGGTETVNTNNCNTHLLIHHRSAAVVPRRTCFWCCKRGLPA

SEQ ID NO: 29 - DNA, 3443 bp, Zea mays
ctgcgtcgccctgcctcgcaatcgcgaatctgtcgagcacctgaggggtcggaggccgagagctagcctagcacgccggcctccgcgcgcgatggaggccagcgccgggctggtggccggctcgcacaaccggaacgagctggtgctgatccggggccacgaggaccccaagccgctgcgggcgctgagcgggcaggtgtgcgagatatgcggcgacgaggtcgggctcacggtggacggcgacctcttcgtcgcctgcaacgagtgcggcttccccgtgtgccggccctgctacgagtacgagcgccgggagggcacgcagaactgcccccagtgcaagacgcgctacaagcgcctcaaggggagcccgagggttgccggggacgatgacgaggaggacatcgacgacctggagcacgagttcaacatcgacgacgagaatcagcagaggcagctggagggcaacatgcagaacagccagatcaccgaggcgatgctgcacggcaggatgagctacgggaggggccccgacgacggcgacggcaacaacaccccgcagatcccgcccatcatcaccggctcccgctccgtgccggtgagcggtgagtttccgattaccaacgggtatggccacggcgaggtctcgtcttccctgcacaagcgcatccatccgtaccctgtgtctgagccagggagtgccaagtgggacgagaagaaagaagtgagctggaaggagaggatggacgactggaagtccaagcagggcatcctcggcggcggcgccgatcccgaagacatggacgccgacgtggcactgaacgacgaggcgaggcagccgctgtcgaggaaggtgtcgatcgcgtcgagcaaggtgaacccgtaccggatggtgatcgtggtgcgtctcgttgtgctcgccttcttcctccggtaccgtatcctgcaccccgtcccggacgccatcgggctgtggctcgtctccatcatctgcgagatctggttcgccatctcctggatcctcgaccagttccccaagtggttccccatcgaccgcgagacgtacctcgaccgcctctccctcaggtacgagagggaaggggagccgtcgctgctgtcggcggtggacctgttcgtgagcacggtggacccgctcaaggagccgccgctggtgaccgccaacaccgtgctctccatcctcgccgtagactaccccgtggacaaggtctcctgctacgtctccgacgacggcgcgtcgatgctgacgttcgagtcgctgtcggagacggccgagttcgcgcgcaagtgggtgcccttctgcaagaagttcggcatcgagccccgcgccccggagttctacttctcgctcaaggtcgactacctcaaggacaaggtgcagcccaccttcgtgcaggagcgccgcgccatgaagagagagtatgaggagttcaaggtccggatcaacgcgctggtggccaaggccatgaaggtgccggcagaggggtggatcatgaaggacggcacgccgtggcccgggaacaacacccgcgaccaccccggcatgatccaggtgttcctgggccacagcggcggccacgacaccgagggcaacgagctgccccgcctcgtgtacgtctcccgtgagaagcgcccgggattccagcaccacaagaaggccggcgccatgaacgctctgattcgcgtctccgccgtgctgaccaacgcgccattcatgctcaacttggactgtgatcactacatcaacaacagcaaggccatccgggaggccatgtgcttcctcatggaccctcaggtcggccggaaggtctgctacgttcagttcccgcagaggttcgacggcatcgacgtgcacgaccgatacgctaacaggaacaccgtcttcttcgacatcaacatgaaggggctggacggcatccaaggcccggtgtacgtcgggacagggtgcgtgttccggcgccaggcgctctacggctacaaccctcccaagggacccaagaggcccaagatggtgacctgcgactgctgcccgtgcttcggccgcaagaagcggaaacacgccaaggacgggctgccggagggcaccgctgatatgggagtagatagcgacaaggagatgctcatgtcccacatgaacttcgagaagcggttcgggcagtccgcggcgttcgtcacgtcgacgctgatggaggaaggcggcgtccctccttcgtcgagccccgccgcgctcctcaaggaggccatccatgtcatcagctgcggctacgaggacaagaccgactgggggctggagctggggtggatctacgggtcgatcacggaggacatcctgacggggttcaagatgcactgccgcgggtggcgctccgtgtactgcatgccgaagcgggcggcgttcaaggggtcggcgccgatcaatctatcggaccgtctcaaccaggtgctccggtgggcgctggggtccgtcgagatcttcttcagccggcacagccccctgctgtacggctacaagaacggcaacctcaagtggctggagcgcttcgcctacatcaacaccaccatctaccccttcacctcgctcccgctgctcgcctactgcaccctccccgccgtctgcctcctcaccggcaagttcatcatgccgtcgattagcacgttcgccagcctcttcttcatcgccctcttcatgtccatcttcgcgacgggcatcctggagatgcggtggagcggggtgagcatcgaggagtggtggaggaacgagcagttctgggtcatcggcggcgtgtccgcgcatctcttcgccgtcgtgcagggcctgctcaaggtcctcgccgggatcgacaccaacttcaccgtcacctccaaggccaccggcgacgaggacgacgagttcgccgagctctacgccttcaagtggaccacgctcctcatcccgcccaccacgctgctcatcattaacgtcatcggcgtcgtggccggcatctccgacgccatcaacaacgggtaccagtcctgggggcccctcttcggcaagctcttcttcgccttctgggtcatcgtccacctctacccgttcctcaaggggctcatggggcgccagaacaggacgcccaccgttgttgtcatctggtccattctgctggcctccatcttctccctgctctgggtcaggatcgaccctttcatcgtcaggaccaagggcccggacgtcaggcagtgtggcatcaattgctgagctgtttattaaggttcaaaattctggagcttgtgcatagggagaaaaaaacaatttagaaattttgtaaggttgttgtgtctgtaatgttatggtacccagaattgtcggacgaggaattgaacaaaggacaaggtttgattgttaaatggcaaaaaaaaaaaaaaaaaaaaaaaaaaaaaaaaaaaaaaaa

SEQ ID NO: 30 - protein, 1052 aa, Zea mays
MEASAGLVAGSHNRNELVLIRGHEDPKPLRALSGQVCEICGDEVGLTVDGDLFVACNECGFPVCRPCYEYERREGTQNCPQCKTRYKRLKGSPRVAGDDDEEDIDDLEHEFNIDDENQQRQLEGNMQNSQITEAMLHGRMSYGRGPDDGDGNNTPQIPPIITGSRSVPVSGEFPITNGYGHGEVSSSLHKRIHPYPVSEPGSAKWDEKKEVSWKERMDDWKSKQGILGGGADPEDMDADVALNDEARQPLSRKVSIASSKVNPYRMVIVVRLVVLAFFLRYRILHPVPDAIGLWLVSIICEIWFAISWILDQFPKWFPIDRETYLDRLSLRYEREGEPSLLSAVDLFVSTVDPLKEPPLVTANTVLSILAVDYPVDKVSCYVSDDGASMLTFESLSETAEFARKWVPFCKKFGIEPRAPEFYFSLKVDYLKDKVQPTFVQERRAMKREYEEFKVRINALVAKAMKVPAEGWIMKDGTPWPGNNTRDHPGMIQVFLGHSGGHDTEGNELPRLVYVSREKRPGFQHHKKAGAMNALIRVSAVLTNAPFMLNLDCDHYINNSKAIREAMCFLMDPQVGRKVCYVQFPQRFDGIDVHDRYANRNTVFFDINMKGLDGIQGPVYVGTGCVFRRQALYGYNPPKGPKRPKMVTCDCCPCFGRKKRKHAKDGLPEGTADMGVDSDKEMLMSHMNFEKRFGQSAAFVTSTLMEEGGVPPSSSPAALLKEAIHVISCGYEDKTDWGLELGWIYGSITEDILTGFKMHCRGWRSVYCMPKRAAFKGSAPINLSDRLNQVLRWALGSVEIFFSRHSPLLYGYKNGNLKWLERFAYINTTIYPFTSLPLLAYCTLPAVCLLTGKFIMPSISTFASLFFIALFMSIFATGILEMRWSGVSIEEWWRNEQFWVIGGVSAHLFAVVQGLLKVLAGIDTNFTVTSKATGDEDDEFAELYAFKWTTLLIPPTTLLIINVIGVVAGISDAINNGYQSWGPLFGKLFFAFWVIVHLYPFLKGLMGRQNRTPTVVVIWSILLASIFSLLWVRIDPFIVRTKGPDVRQCGINC

SEQ ID NO: 31 - DNA, 36 bp, Artificial Sequence
  Sal-A20 oligonucleotide
tcgacccacgcgtccgaaaaaaaaaaaaaaaaaaaa

SEQ ID NO: 32 - DNA, 28 bp, Artificial Sequence
  GSP1 forward primer
tacgatgagtacgagaggtccatgctca

SEQ ID NO: 33 - DNA, 26 bp, Artificial Sequence
  GSP2 reverse primer
ggcaaaagcccagatgcgagatagac

SEQ ID NO: 34 - DNA, 32 bp, Artificial Sequence
  Mu TIR primer
agagaagccaacgccawcgcctcyatttcgtc

SEQ ID NO: 35 - DNA, 9 bp, Zea mays
tggcggccg

SEQ ID NO: 36 - DNA, 9 bp, Zea mays
tctgaaatg

SEQ ID NO: 37 - DNA, 9 bp, Zea mays
gcccacaag

SEQ ID NO: 38 - DNA, 9 bp, Zea mays
catcctggt

SEQ ID NO: 39 - DNA, 9 bp, Zea mays
gtgttcttc

SEQ ID NO: 40 - DNA, 9 bp, Zea mays
gccatgtgg

SEQ ID NO: 41 - DNA, 3568 bp, Zea mays
  misc_feature 3487: n = A,T,C or G
gtcgacccacgcgtccggagctcgtcgtcatccgccgcgatggcgagccagggccgaagcccatggaccagcggaacggccaggtgtgccagatttgcggcgacgacgtggggcgcaaccccgacggggagcctttcgtggcctgcaacgagtgcgccttccccatctgccgggactgctacgagtacgagcgccgcgagggcacgcagaactgcccccagtgcaagacccgcttcaagcgcttcaaggggtgcgcgcgcgtgcccggggacgaggaggaggacggcgtcgacgacctggagaacgagttcaactggagcgacaagcacgactcccagtacctcgccgagtccatgctccacgcccacatgagctacggccgcggcgccgacctcgacggcgtgccgcagccattccaccccatccccaatgttcccctcctcaccaacggacagatggtcgatgacatcccgccggaccagcacgcccttgtgccctcgttcgtgggtggcggggggaagaggattcaccctctcccgtacgcggatcccaaccttcctgtgcaaccgaggtctatggacccttccaaggatctcgccgcatatggctacgggagcgtagcatggaaggagaggatggagagctggaagcagaagcaggagaggatgcaccagacgaggaacgatggcggcggcgatgatggtgatgatgcagatctaccactaatggatgaagctagacagccattgtccagaaagatcccgcttccttcaagccaaatcaacccctataggatgattataataattcggctagtggttttgtgtttcttcttccactaccgagtgatgcatccggtgcctgatgcatttgctttatggctcatatctgtgatctgtgaaatttggtttgccatgtcttggattcttgaccagtttccaaagtggtttcctatcgagagggaaacctatcttgaccggctgagtttaaggtttgacaaggaagggcatccttctcaactcgcccctgttgatttctttgtcagtacggttgatcccttgaaggaacctccattggtcactgctaatactgttctatctatcctttcggtggattatccagttgataaggtttcatgctacgtttctgatgatggtgctgccatgctgacatttgaagcattgtctgaaacatctgaatttgcaaagaaatgggttcctttctgcaaaagatatagccttgagcctcgtgctccagagtggtacttccaacagaagatagactacctgaaagacaaggtggcgccaaactttgttagagaacggagagcaatgaagagagagtatgaggaattcaaggtcagaatcaatgccttggttgctaaagcccaaaaggttcctgaggaaggatggacaatgcaggatggaactccatggcccggaaataatgtccgtgatcatcctggaatgattcaggttttccttggtcaaagtggtggccatgatgtggaaggaaatgagctgcctcgattggtttatgtttcaagagaaaaacggccaggctacaaccatcacaagaaggctggtgctatgaatgcattggtccgagtctctgctgtactaactaatgctccttatttgctgaacttggattgtgatcactatatcaataatagtaaggctataaaggaagcaatgtgttttatgatggatcctttgcttggaaagaaagtttgctatgtgcagtttcctcaaagatttgatgggattgatcgccatgatcgatatgctaacagaaatgttgtctttttcgatatcaacatgaaaggtttggatggtatccagggcccaatttatgtgggtactggatgtgtcttcagaaggcaggcattatatggctacgatgctcccaaaacaaagaagccaccatcaagaacttgcaactgctggccaaagtggtgcatttgctgttgctgttttggtaacaggaagaccaagaagaagaccaagacctctaaacctaaatttgagaagataaagaaactttttaagaaaaaggaaaatcaagcccctgcatatgctcttggtgaaattgatgaagccgctccaggagctgaaaatgaaaaggctagtattgtaaatcaacagaagttggaaaagaaatttggccagtcttcagtttttgttgcatccacacttcttgagaatggtggaaccctgaagagtgccagtccagcttctcttctgaaggaagctatacatgtcatcagttgtggatatgaagacaaaacaggctggggaaaagatattggttggatttatggatcagtcacagaagatattcttactgggtttaagatgcactgccatggttggcggtcaatttactgcatacctaaacgggccgccttcaaaggttccgcacctctcaatctttccgatcgttttcaccaggttcttcggtgggctcttggttcaattgaaattttgttcagcaaccactgccctctctggtatgggtatggtggtggactaaagttcctggaaaggttttcgtacattaactccatcgtatacccttggacatctatcccgctcttggcctattgcacattgcctgccatctgcttgctgacagggaaatttatcacgccagagcttaacaatgttgccagcctctggttcatgtcacttttcatctgcatttttgctacgagcatcctggaaatgagatggagtggtgtaggcatcgatgactggtggagaaacgagcagttttgggtcattggaggcgtgtcttcacatctctttgctgtgttccagggactcctcaaggtcatagctggtgtagacacgagcttcactgtgacatccaagggcggagacgacgaggagttctcagagctgtacacattcaaatggacgacccttctgatacctccgacaaccctgctcctactgaacttcattggagtggtagctggcatctccaatgcgatcaacaacggatatgaatcatggggccccctgttcgggaagctcttctttgcattttgggtgatcgtccatctttacccgttcctcaagggtctggttgggaggcagaacaggacgccaacgattgtcattgtctggtccatcctcctggcttcgatcttctcgctgctttgggtccggatcgacccgttccttgcgaaggatgatggtcccctgttggaggagtgtggtctggattgcaactaggaggtcagcacgtggacttccccgtcagtgtgtggtcgaagaagtatttttgcagatgttttgtgcccatatttctttactcaatttttgtccctctgtagattgaaacaaggggtgaaggggaaaaaaagtacttgtatttcttttgttccatggtggtggtggtggtgggcggctcagcctcgtgagtgcaatattgggcaaaccggaggttgcggcaaccttgtgcagttcgtccacgaatntactagggatgatcgcgaccaatcaatcaatcgatgaccgagttcaattgttcaaaaaaaaaaaaaaaaaagggcggccgc

SEQ ID NO: 42 - protein, 1059 aa, Zea mays
MDQRNGQVCQICGDDVGRNPDGEPFVACNECAFPICRDCYEYERREGTQNCPQCKTRFKRFKGCARVPGDEEEDGVDDLENEFNWSDKHDSQYLAESMLHAHMSYGRGADLDGVPQPFHPIPNVPLLTNGQMVDDIPPDQHALVPSFVGGGGKRIHPLPYADPNLPVQPRSMDPSKDLAAYGYGSVAWKERMESWKQKQERMHQTRNDGGGDDGDDADLPLMDEARQPLSRKIPLPSSQINPYRMIIIIRLVVLCFFFHYRVMHPVPDAFALWLISVICEIWFAMSWILDQFPKWFPIERETYLDRLSLRFDKEGHPSQLAPVDFFVSTVDPLKEPPLVTANTVLSILSVDYPVDKVSCYVSDDGAAMLTFEALSETSEFAKKWVPFCKRYSLEPRAPEWYFQQKIDYLKDKVAPNFVRERRAMKREYEEFKVRINALVAKAQKVPEEGWTMQDGTPWPGNNVRDHPGMIQVFLGQSGGHDVEGNELPRLVYVSREKRPGYNHHKKAGAMNALVRVSAVLTNAPYLLNLDCDHYINNSKAIKEAMCFMMDPLLGKKVCYVQFPQRFDGIDRHDRYANRNVVFFDINMKGLDGIQGPIYVGTGCVFRRQALYGYDAPKTKKPPSRTCNCWPKWCICCCCFGNRKTKKKTKTSKPKFEKIKKLFKKKENQAPAYALGEIDEAAPGAENEKASIVNQQKLEKKFGQSSVFVASTLLENGGTLKSASPASLLKEAIHVISCGYEDKTGWGKDIGWIYGSVTEDILTGFKMHCHGWRSIYCIPKRAAFKGSAPLNLSDRFHQVLRWALGSIEILFSNHCPLWYGYGGGLKFLERFSYINSIVYPWTSIPLLAYCTLPAICLLTGKFITPELNNVASLWFMSLFICIFATSILEMRWSGVGIDDWWRNEQFWVIGGVSSHLFAVFQGLLKVIAGVDTSFTVTSKGGDDEEFSELYTFKWTTLLIPPTTLLLLNFIGVVAGISNAINNGYESWGPLFGKLFFAFWVIVHLYPFLKGLVGRQNRTPTIVIVWSILLASIFSLLWVRIDPFLAKDDGPLLEECGLDCN

SEQ ID NO: 43 - DNA, 25 bp, Artificial Sequence
  amplicon
atggaccagcggaacggccaggtgt

SEQ ID NO: 44 - DNA, 25 bp, Artificial Sequence
  amplicon
ctagttgcaatccagaccacactcc

SEQ ID NO: 45 - DNA, 3725 bp, Zea mays
gcagcagcagcaccaccactgcgcggcattgcagcgagcaagcgggagggatctggggcatggtggcggtcgctgccgctgccgctcggatctagagggccgcacgggctgattgccctccgccggcctcgtcggtgtcggtggagtgtgaatcggtgtgtgtaggaggagcgcggagatggcggccaacaaggggatggtggcaggctctcacaaccgcaacgagttcgtcatgatccgccacgacggcgacgcgcctgtcccggctaagcccacgaagagtgcgaatgggcaggtctgccagatttgtggcgacactgttggcgtttcagccactggtgatgtctttgttgcctgcaatgagtgtgccttccctgtctgccgcccttgctatgagtacgagcgcaaggaagggaaccaatgctgccctcagtgcaagactagatacaagagacagaaaggtagccctcgagttcatggtgatgatgaggaggaagatgttgatgacctggacaatgaattcaactataagcaaggcaatgggaagggcccagagtggcagcttcaaggagatgacgctgatctgtcttcatctgctcgccatgacccacaccatcggattccacgccttacaagtggacaacagatatctggagagatccctgatgcatcccctgaccgtcattctatccgcagtccaacatcgagctatgttgatccaagcgttccagttcctgtgaggattgtggacccctcgaaggacttgaattcctatgggcttaatagtgttgactggaaggaaagagttgagagctggagggttaaacaggacaaaaatatgttgcaagtgactaataaatatccagaggctagaggagacatggaggggactggctcaaatggagaagatatgcaaatggttgatgatgcacgcctacctttgagccgcattgtgccaatttcctcaaaccagctcaacctttaccggatagtaatcattctccgtcttatcatcctgtgcttcttcttccaatatcgtatcagtcatccagtgcgtaatgcttatggattgtggctagtatctgttatctgtgaggtctggtttgccttgtcctggcttctagatcagttcccaaaatggtatccaatcaaccgtgagacatatctcgacaggcttgcattgaggtatgatagagagggagagccatcacagctggctcccattgatgtctttgtcagtacagtggatccattgaaggaacctccactgatcacagccaacactgttttgtccattcttgctgtggattaccctgttgacaaagtgtcatgctatgtttctgatgatggctcagctatgctgacttttgagtctctctctgaaactgccgaatttgctagaaagtgggttcccttttgtaagaagcacaatattgaaccaagagctccagaattttactttgctcaaaaaatagattacctgaaggacaaaattcaaccttcatttgttaaggaaagacgagcaatgaagagagagtatgaagaattcaaaataagaatcaatgcccttgttgccaaagcacagaaagtgcctgaagaggggtggaccatggctgatggaactgcttggcctgggaataaccctagggaccatcctggcatgattcaggtgttcttggggcacagtggtgggcttgacactgatggaaatgaattaccacgtcttgtctatgtctctcgtgaaaagagaccaggctttcagcatcacaagaaggctggtgcaatgaatgcactgattcgtgtatctgctgtgctgacaaatggtgcctatcttctcaatgtggattgtgaccattacttcaatagcagcaaagctcttagagaagcaatgtgcttcatgatggatccagctctaggaaggaaaacttgttatgtacaatttccacaaagatttgatggcattgacttgcacgatcgatatgctaataggaacatagtcttctttgatatcaacatgaaaggtctagatggcattcagggtccagtctatgtgggaacaggatgctgtttcaataggcaggctttgtatggatatgatcctgttttgactgaagctgatctggaacctaacattgttgttaagagctgctgtggtagaaggaagagaaagaacaagagttatatggatagtcaaagccgtattatgaagagaacagaatcttcagctcccatctttaacatggaagacatcgaggagggtattgaaggttatgaggatgaaaggtcagtgcttatgtcccagaggaaattggagaaacgctttggtcagtctccaatcttcattgcatccacctttatgactcaaggtggcataccaccttcaacaaacccagcttctctactgaaggaagctatccatgttatcagctgtgggtacgaggacaaaactgaatggggaaaagagattggctggatctatggttcagttacagaggatattctgactgggtttaaaatgcatgcaagaggctggcaatcaatctactgcatgccaccacgaccttgtttcaagggttctgcaccaatcaatctttctgatcgtcttaatcaggtgctccgttgggctcttgggtcagtggaaattctgcttagcagacattgtcctatatggtatggctacaatgggcgattgaagcttttggagaggctggcttacattaacaccattgtttatccaatcacatctgttccgcttatcgcctattgtgtgcttcctgctatctgtcttcttaccaataaatttatcattcctgagattagtaattatgctggaatgttcttcattcttctttttgcctccattttcgcaactggtatattggagctcagatggagtggtgttggcattgaagattggtggagaaatgagcagttttgggttattggtggcacctctgcccatctcttcgcggtgttccagggtctgctgaaagtgttggctgggattgataccaacttcacagttacctcaaaggcatctgatgaggatggcgactttgctgagctatatgtgttcaagtggaccagtttgctcatccctccgaccactgttcttgtcattaacctggtcggaatggtggcaggaatttcgtatgccattaacagcggctaccaatcctggggtccgctctttggaaagctgttcttctcgatctgggtgatcctccatctctaccccttcctcaagggtctcatgggcaggcagaaccgcacgccaacaatcgtcatcgtttggtccatcctccttgcgtctatcttctccttgctgtgggtgaagatcgatcctttcatctccccgacacagaaagctgccgccttggggcaatgtggtgtgaactgctgatccagattgtgactcttatctgaagaggctcagccaaagatctgccccctcgtgtaaatacctgagggggctagatgggaattttttgttgtagatgaggatggatctgcatccaagttatgcctctgtttattagcttcttcggtgccggtgctgctgcagacaatcatggagcctttctaccttgcttgtagtgctggccagcagcgtaaattgtgaattctgcatttttttatacgtggtgtttattgttttagagtaaattatcatttgtttgaggtaactattcacacgaactatatggcaatgctgttatttaaaa

SEQ ID NO: 46 - protein, 1074 aa, Zea mays
MAANKGMVAGSHNRNEFVMIRHDGDAPVPAKPTKSANGQVCQICGDTVGVSATGDVFVACNECAFPVCRPCYEYERKEGNQCCPQCKTRYKRQKGSPRVHGDDEEEDVDDLDNEFNYKQGNGKGPEWQLQGDDADLSSSARHDPHHRIPRLTSGQQISGEIPDASPDRHSIRSPTSSYVDPSVPVPVRIVDPSKDLNSYGLNSVDWKERVESWRVKQDKNMLQVTNKYPEARGDMEGTGSNGEDMQMVDDARLPLSRIVPISSNQLNLYRIVIILRLIILCFFFQYRISHPVRNAYGLWLVSVICEVWFALSWLLDQFPKWYPINRETYLDRLALRYDREGEPSQLAPIDVFVSTVDPLKEPPLITANTVLSILAVDYPVDKVSCYVSDDGSAMLTFESLSETAEFARKWVPFCKKHNIEPRAPEFYFAQKIDYLKDKIQPSFVKERRAMKREYEEFKIRINALVAKAQKVPEEGWTMADGTAWPGNNPRDHPGMIQVFLGHSGGLDTDGNELPRLVYVSREKRPGFQHHKKAGAMNALIRVSAVLTNGAYLLNVDCDHYFNSSKALREAMCFMMDPALGRKTCYVQFPQRFDGIDLHDRYANRNIVFFDINMKGLDGIQGPVYVGTGCCFNRQALYGYDPVLTEADLEPNIVVKSCCGRRKRKNKSYMDSQSRIMKRTESSAPIFNMEDIEEGIEGYEDERSVLMSQRKLEKRFGQSPIFIASTFMTQGGIPPSTNPASLLKEAIHVISCGYEDKTEWGKEIGWIYGSVTEDILTGFKMHARGWQSIYCMPPRPCFKGSAPINLSDRLNQVLRWALGSVEILLSRHCPIWYGYNGRLKLLERLAYINTIVYPITSVPLIAYCVLPAICLLTNKFIIPEISNYAGMFFILLFASIFATGILELRWSGVGIEDWWRNEQFWVIGGTSAHLFAVFQGLLKVLAGIDTNFTVTSKASDEDGDFAELYVFKWTSLLIPPTTVLVINLVGMVAGISYAINSGYQSWGPLFGKLFFSIWVILHLYPFLKGLMGRQNRTPTIVIVWSILLASIFSLLWVKIDPFISPTQKAAALGQCGVNC

SEQ ID NO: 47 - DNA, 25 bp, Artificial Sequence
  amplicon
atggcggccaacaaggggatggtgg

SEQ ID NO: 48 - DNA, 25 bp, Artificial Sequence
  amplicon
tcagcagttcacaccacattgcccc

SEQ ID NO: 49 - DNA, 3969 bp, Zea mays
cttctccctcgtcggtgcggcgtggcgcggctcggcgttcggtgagaaaccactcgggggatgaggatctgctgctagagtgagaggagctacggtcagtatcctctgccttcgtcggcggcggaagtggaggggaggaagcgatggaggcgagcgccgggctggtggccggctcccacaaccgcaacgagctcgtcgtcatccgccgcgacggcgatcccgggccgaagccgccgcgggagcagaacgggcaggtgtgccagatttgcggcgacgacgtcggccttgcccccggcggggaccccttcgtggcgtgcaacgagtgcgccttccccgtctgccgggactgctacgaatacgagcgccgggagggcacgcagaactgcccccagtgcaagactcgatacaagcgcctcaagggctgccaacgtgtgaccggtgacgaggaggaggacggcgtcgatgacctggacaacgagttcaactgggacggccatgactcgcagtctgtggccgagtccatgctctacggccacatgagctacggccgtggaggtgaccctaatggcgcgccacaagctttccagctcaaccccaatgttccactcctcaccaacgggcaaatggtggatgacatcccaccggagcagcacgcgctggtgccttctttcatgggtggtgggggaaagaggatacatccccttccttatgcggatcccagcttacctgtgcaacccaggtctatggacccatccaaggatcttgctgcatatgggtatggtagtgttgcttggaaggaacggatggagaattggaagcagagacaagagaggatgcaccagacggggaatgatggtggtggtgatgatggtgacgatgctgatctaccactaatggatgaagcaagacaacaactgtccaggaaaattccacttccatcaagccagattaatccatataggatgattatcattattcggcttgtggttttggggttcttcttccactaccgagtgatgcatccggtgaatgatgcatttgctttgtggctcatatctgttatctgtgaaatctggtttgccatgtcttggattcttgatcaattcccaaagtggttccctattgagagagagacttacctagaccggctgtcactgaggttcgacaaggaaggccagccatctcaacttgctccaattgatttctttgtcagtacggttgatcccttaaaggaacctcctttggtcacaacaaatactgttctatctatcctttcggtggattatcctgttgataaggtttcttgctatgtttctgatgatggtgctgcaatgctaacgtttgaagcattatctgaaacatctgaatttgcaaagaaatgggttcctttctgcaaacggtacaatattgaacctcgcgctccagagtggtacttccaacagaagatagactacttgaaagacaaggtggcagcaaactttgttagggagaggagagcaatgaagagagagtatgaggaattcaaggtgagaatcaatgccttagttgccaaagcccagaaagttcctgaagaaggatggacaatgcaagatggaaccccctggcctggaaacaatgttcgtgatcatcctggaatgattcaggtcttccttggccaaagcggaggccttgactgtgagggaaatgaactgccacgattggtttatgtttctagagagaaacgaccaggctataaccatcataagaaagctggtgctatgaatgcattggtccgagtctctgctgtactaacaaatgctccatatttgttaaacttggattgtgatcactacatcaacaacagcaaggctataaaggaagcaatgtgttttatgatggaccctttactaggaaagaaggtttgctatgtacagttccctcaaagatttgatgggattgatcgccatgaccgatatgctaaccggaatgttgtcttttttgatatcaacatgaaaggtttggatggtattcagggtccaatttatgttggtactggatgtgtatttagaaggcaggcattatatggttatgatgcccccaaaacaaagaagccaccatcaaggacttgcaactgctggcccaagtggtgcttttgctgttgctgctttggcaataggaagcaaaagaagactaccaaacccaaaacagagaagaaaaagttattatttttcaagaaagaagagaaccaatcccctgcatatgctcttggtgaaattgacgaagctgctccaggagctgagaatgaaaaggccggtattgtaaatcaacaaaaattagaaaagaaatttggccaatcttctgtttttgttacatccacacttctcgagaatggtggaaccttgaagagtgcaagtcctgcttctcttttgaaagaagctatacatgtcattagttgtggttatgaagacaagacagactggggaaaagagattggctggatctatggatcagttacagaagatattctaactggtttcaagatgcattgtcatggttggcggtcaatttactgcatacctaaacgggttgcattcaaaggttctgcacctctgaatctttcagatcgtcttcaccaggtgcttcggtgggctcttgggtctattgagatcttcttcagcaatcattgccctctttggtatgggtatggtggcggtctgaaatttttggaaagattttcctacatcaactccatcgtgtatccttggacatctattcccctcttggcttactgtacattgcctgccatctgtttattgacagggaaatttatcactccagagctgaataatgttgccagcctgtggttcatgtcactttttatctgcatttttgctacgagcatcctagaaatgagatggagtggtgttggaattgatgactggtggaggaatgagcagttctgggtcattggaggtgtgtcctcacacctctttgctgtgttccagggacttctcaaggtcatagctggtgttgatacaagcttcaccgtgacatcaaagggtggagatgatgaggagttctcagagctatatacattcaaatggactaccttattgatacctcctaccaccttgcttctattgaacttcattggtgtggtcgctggcgtttcaaatgcgatcaataacggatatgagtcatggggccccctctttgggaagctattctttgcattttgggtgattgtccatctttatccctttctcaaaggtttggttggaaggcaaaacaggacaccaacgattgtcatcgtctggtccattctgctggcttcaatcttctcgctcctttgggttcggattgatcctttccttgcgaaggatgatggtccgcttcttgaggagtgtggtttggattgcaactaggatgtcagtgcatcagctcccccaatctgcatatgcttgaagtatattttctggtgtttgtccccatattcagtgtctgtagataagagacatgaaatgtcccaagtttcttttgatccatggtgaacctacttaatatctgagagatatactgggggaaaatggaggctgcggcaatccttgtgcagttgggccgtggaatacagcatatgcaagtgtttgattgtgcagcattctttattacttggtcgcaatatagatgggctgagccgaacagcaaggtattttgattctgcactgctcccgtgtacaaacttggttctcaataaggcaggcaggaatgcatctgccagtggaacagagcaacctgcacattatttatgtatgcctgttcattggagggcttgttcattacatgttcgtctatactagaaaaaacagaatattagcattaatctatagttaattaaagtatgtaaatgcgcctgttttttgttgtgtactgtaatcatctgagttggttttgtgaaaaaaaaaaaaaaaaaaaaaaaaaaaaaaaaaaaaa

SEQ ID NO: 50 - protein, 1086 aa, Zea mays
MEASAGLVAGSHNRNELVVIRRDGDPGPKPPREQNGQVCQICGDDVGLAPGGDPFVACNECAFPVCRDCYEYERREGTQNCPQCKTRYKRLKGCQRVTGDEEEDGVDDLDNEFNWDGHDSQSVAESMLYGHMSYGRGGDPNGAPQAFQLNPNVPLLTNGQMVDDIPPEQHALVPSFMGGGGKRIHPLPYADPSLPVQPRSMDPSKDLAAYGYGSVAWKERMENWKQRQERMHQTGNDGGGDDGDDADLPLMDEARQQLSRKIPLPSSQINPYRMIIIIRLVVLGFFFHYRVMHPVNDAFALWLISVICEIWFAMSWILDQFPKWFPIERETYLDRLSLRFDKEGQPSQLAPIDFFVSTVDPLKEPPLVTTNTVLSILSVDYPVDKVSCYVSDDGAAMLTFEALSETSEFAKKWVPFCKRYNIEPRAPEWYFQQKIDYLKDKVAANFVRERRAMKREYEEFKVRINALVAKAQKVPEEGWTMQDGTPWPGNNVRDHPGMIQVFLGQSGGLDCEGNELPRLVYVSREKRPGYNHHKKAGAMNALVRVSAVLTNAPYLLNLDCDHYINNSKAIKEAMCFMMDPLLGKKVCYVQFPQRFDGIDRHDRYANRNVVFFDINMKGLDGIQGPIYVGTGCVFRRQALYGYDAPKTKKPPSRTCNCWPKWCFCCCCFGNRKQKKTTKPKTEKKKLLFFKKEENQSPAYALGEIDEAAPGAENEKAGIVNQQKLEKKFGQSSVFVTSTLLENGGTLKSASPASLLKEAIHVISCGYEDKTDWGKEIGWIYGSVTEDILTGFKMHCHGWRSIYCIPKRVAFKGSAPLNLSDRLHQVLRWALGSIEIFFSNHCPLWYGYGGGLKFLERFSYINSIVYPWTSIPLLAYCTLPAICLLTGKFITPELNNVASLWFMSLFICIFATSILEMRWSGVGIDDWWRNEQFWVIGGVSSHLFAVFQGLLKVIAGVDTSFTVTSKGGDDEEFSELYTFKWTTLLIPPTTLLLLNFIGVVAGVSNAINNGYESWGPLFGKLFFAFWVIVHLYPFLKGLVGRQNRTPTIVIVWSILLASIFSLLWVRIDPFLAKDDGPLLEECGLDCN

SEQ ID NO: 51 - DNA, 25 bp, Artificial Sequence
  amplicon
atggaggcgagcgccgggctggtgg

SEQ ID NO: 52 - DNA, 25 bp, Artificial Sequence
  amplicon
ctagttgcaatccaaaccacactcc